(19) United States
(12) Patent Application Publication
(10) Pub. No.: US 2023/0144855 A1
(43) Pub. Date: May 11, 2023

(54) METHODS AND COMPOSITIONS FOR INDUCING FETAL HEMOGLOBIN

(71) Applicant: Flagship Pioneering Innovations VI, LLC, Cambridge, MA (US)

(72) Inventors: Mauricio CORTES, Wellesley, MA (US); Xiaoji SUN, Cambridge, MA (US); Anthony Joseph MONTI, Wrentham, MA (US); Nicholas McCartney PLUGIS, Boston, MA (US)

(21) Appl. No.: 17/901,371

(22) Filed: Sep. 1, 2022

Related U.S. Application Data
(60) Provisional application No. 63/239,823, filed on Sep. 1, 2021.
(60) Provisional application No. 63/284,979, filed on Dec. 1, 2021.
(60) Provisional application No. 63/239,826, filed on Sep. 1, 2021.
(60) Provisional application No. 63/284,986, filed on Dec. 1, 2021.

(51) Int. Cl. A61K 31/519; A61P 7/06; A61K 31/17; G01N 33/50; A61K 31/573

(52) U.S. Cl. CPC A61K 31/519; A61P 7/06; A61K 31/17; G01N 33/5023; A61K 31/573

(57) ABSTRACT

The present disclosure provides methods for increasing the quantity and/or the ratios of erythroblasts, reticulocytes, and/or erythrocytes, or progenitors thereof, in which any of these cells express HbF (e.g. HbF⁺ and/or HbF^{high} cells). The present disclosure further provides methods for treating diseases or disorders characterized by, for example, oxygen delivery deficiencies and/or reduced expression and/or activity of hemoglobin. Such diseases, without limitation, are mitigated by therapeutic reactivation of HbF.

### CROSS-REFERENCE TO RELATED APPLICATIONS

This application claims priority to and benefit of U.S. Provisional Patent Application No. 63/239,823 filed on Sep. 1, 2021, U.S. Provisional Patent Application No. 63/284,979 filed on Dec. 1, 2021, U.S. Provisional Patent Application No. 63/239,826 filed on Sep. 1, 2021, and U.S. Provisional Patent Application No. 63/284,986 filed on Dec. 1, 2021, the contents of all of which are hereby incorporated by reference in their entireties.

### BACKGROUND

An understanding of cellular mechanisms relating to development of erythroblasts, reticulocytes, and erythrocytes, and their lineages, as well as methods and agents for directing changes in development of erythroblasts, reticulocytes, and erythrocytes comprising fetal hemoglobin, may be useful for treating diseases or disorders, including those underscored by abnormal amounts or ratio of various cell types. Currently, there is an unmet need for such methods and agents that can be used for the treatment of such diseases and disorders.

### SUMMARY

In aspects and embodiments, there is provided a method for directing a change in cell state of a progenitor cell comprising, contacting a population of cells comprising a progenitor cell with at least one perturbagen selected from Table 3, or a variant thereof, and wherein the progenitor cell is a non-lineage committed CD34+ cell.

In embodiments, the at least one perturbagen is capable of altering a gene signature in the progenitor cell.

In aspects and embodiments, there is provided a method for directing a change in cell state of a progenitor cell, comprising, contacting a population of cells comprising a progenitor cell with at least one perturbagen capable of altering a gene signature in the progenitor cell, wherein altering the gene signature comprises an increase in expression and/or activity in the progenitor cell of one or more genes selected from the genes designated as an “up” gene in the gene directionality column of Table 1a and/or Table 2 and/or a decrease in expression and/or activity in the progenitor cell of one or more genes selected from the genes designated as a “down” gene in the gene directionality column of Table 1a and/or Table 2 and/or an increase in expression and/or activity in the progenitor cell of one or more genes selected from Table 1b and/or a decrease in expression and/or activity in the progenitor cell of one or more genes selected from Table 1b and wherein the progenitor cell is a non-lineage committed CD34+ cell.

In aspects and embodiments, there is provided a method for directing a change in cell state of a progenitor cell, comprising, contacting a population of cells comprising a progenitor cell with at least one perturbagen selected from Table 3, or a variant thereof, and capable of altering a gene signature in the progenitor cell, wherein altering the gene signature comprises an increase in expression and/or activity in the progenitor cell of one or more genes selected from the genes designated as an “up” gene in the gene directionality column of Table 1a and/or Table 2 and/or a decrease in expression and/or activity in the progenitor cell of one or more genes selected from the genes designated as a “down” gene in the gene directionality column of Table 1a and/or Table 2 and/or an increase in expression and/or activity in the progenitor cell of one or more genes selected from Table 1b and/or a decrease in expression and/or activity in the progenitor cell of one or more genes selected from Table 1b and wherein the progenitor cell is a non-lineage committed CD34+ cell.

In embodiments, altering the gene signature comprises an increase in expression and/or activity in the progenitor cell of a network module designated in the network module column of Table 1a and/or Table 1b.

In embodiments, the change in cell state provides an increase in the number of one or more of proerythroblasts, early erythroblasts, intermediate erythroblasts, late erythroblasts, reticulocytes, and erythrocytes.

In embodiments, the change in cell state provides an increase in F cells.

In embodiments, one or more of the proerythroblasts, early erythroblasts, intermediate erythroblasts, late erythroblasts, reticulocytes, and erythrocytes expresses HBG1 and/or HBG2

In embodiments, the increase in the number of erythrocytes comprising HbF is relative to the number of erythrocytes obtained from a population of progenitor cells that is not contacted with the at least one perturbagen, or relative to the population of progenitor cells prior to contacting with the at least one perturbagen.

In embodiments, the change in cell state provides an increase in the number of erythrocytes comprising HbF. In embodiments, the number of progenitor cells is decreased. In embodiments, the number of progenitor cells is increased.

In embodiments, the number of proerythroblasts, BFU-E cells, CFU-E cells, early erythroblasts, intermediate erythroblasts, late erythroblasts, reticulocytes, and/or erythrocytes is increased after contacting the population of cells comprising a CD34+ cell with the at least one perturbagen, wherein one or more of the proerythroblasts, early erythroblasts, intermediate erythroblasts, late erythroblasts, reticulocytes, and erythrocytes comprise HbF.

In embodiments, the number of proerythroblasts is decreased. In embodiments, the number of proerythroblasts is increased. In embodiments, the number of F cells is increased. In embodiments, at least one perturbagen selected from Table 3, or a variant thereof, comprises at least 2, at least 3, at least 4, or at least 5 perturbagens selected from Table 3, or variants thereof.

In embodiments, at least one perturbagen is used in combination with one or more additional therapeutic agents. In embodiments, the additional therapeutic agent is hydroxyurea (HU).

In embodiments, one or more genes selected from the genes designated as an “up” gene in the gene directionality column of Table 1a comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, 81 or more, 82 or more, 83 or more, 84 or more, 85 or more, 86 or more, 87 or more, 88 or more, 89 or more, 90 or more, 91 or more, 92 or more, 93 or more, 94 or more, 95 or more, 96 or more, 97 or more, 98 or more, 99 or more, 100 or more, 101 or more, 102 or more, 103 or more, 104 or more, or 105 or more genes selected from the genes designated as an “up” gene in the gene directionality column of Table 1a.

In embodiments, one or more genes selected from the genes designated as an “up” gene in the gene directionality column of Table 1a comprises at least one of DDIT4, EPRS, MTHFD2, EIF4EBP1, AARS, ABCC5, PHGDH, TUBB6, LSM6, EIF4G1, RNF167, CD320, CTNNAL1, GADD45A, PTK2, CFLAR, IGF2BP2, CDK1, CDC45, CDCA4, MELK, HAT1, PAK1, TSPAN6, TIMM17B, KDM5A, UBE3B, RPS5, PAICS, RPIA, KDELR2, PNP, CAST, H2AFV, ATP11B, CTNND1, ORC1, FDFT1, CDKN1B, INSIG1, IGF1R, TRAP1, TSTA3, SUZ12, CDK4, HMGCS1, LAP3, TBPL1, FAH, CCP110, APOE, IGF2R, DYRK3, MYBL2, APP, DNMT1, SMC3, HTATSF1, CAT, ACAT2, HK1, PSMD4, CLTC, MAP4K4, PROS1, DLD, SDHB, GNAS, COPS7A, MPC2, HEBP1, BLVRA, ID2, SCAND1, ETFB, MRPS16, PIN1, TRAK2, AMDHD2, PLEKHJ1, BZW2, PCNA, WDR61, RFC5, OXA1L, MCM3, CEP57, PSMF1, POLR2K, PSMD2, ATP6V1D, PSMD9, AKAP8L, GRN, SPAG7, ENOSF1, PCK2, PCCB, NOLC1, EBNA1BP2, CD58, RFC2, ASAH1, LAGE3, AKR7A2, and RSU1.

In embodiments, one or more genes selected from the genes designated as a “down” gene in the gene directionality column of Table 1a comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, or 26 or more genes selected from the genes designated as a “down” gene in the gene directionality column of Table 1a.

In embodiments, one or more genes selected from the genes designated as a “down” gene in the gene directionality column of Table 1a comprises at least one of NUCB2, XBP1, CCNB1, CDC20, PLK1, CDK6, ITGB1BP1, CCNE2, PTPN6, CBR1, HLA-DRA, MAP7, SOX4, CASP3, DNAJB6, HOXA10, IL1B, ICAM3, ADGRG1, HLA-DMA, PDLIM1, PSMB8, EPB41L2, RPL39L, PYGL, CYB561, and HOMER2.

In embodiments, one or more genes selected from the genes designated as an “up” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, o 16 or more, 17 or more, 18 or more, or 19 or more genes selected from the genes designated as an “up” gene in the gene directionality column of Table 2.

In embodiments, one or more genes selected from the genes designated as an “up” gene in the gene directionality column of Table 2 comprises at least one of HMGA1, KLF1, KLF6, SREBF1, NFE2, ARID3A, GFI1B, KLF13, MLXIP, E2F8, MYBL2, HSF1, GMEB1, NFX1, TGIF1, KLF3, SP1, CENPX, HES6, and LIN28B.

In embodiments, one or more genes selected from the genes designated as a “down” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, or 19 or more genes selected from the genes designated as a “down” gene in the gene directionality column of Table 2.

In embodiments, one or more genes selected from the genes designated as a “down” gene in the gene directionality column of Table 2 comprises at least one of HOXA10, XBP1, SOX4, ZNF385D, NFIC, BATF, HHEX, RARG, KDM5B, ZFX, SPI1, TEAD4, SATB1, NFIX, PLAGL1, MEF2C, ZBTB1, HOXA9, THAP5, and ZFP57.

In embodiments, one or more genes selected from Table 1b comprises 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or 41 or more, or 42 or more, or 43 or more, or 44 or more, or 45 or more, or 46 or more, or 47 or more, or 48 or more, or 49 or more, 50 or more, 51 or more, or 52 or more, or 53 or more, or 54 or more, or 55 or more, or 56 or more, or 57 or more, or 58 or more, or 59 or more, 60 or more, or 61 or more, or 62 or more, or 63 or more, or 64 or more, or 65 or more, or 66 or more, or 67 or more, or 68 or more, or 69 or more, 70 or more, or 71 or more, or 72 or more, or 73 or more, or 74 or more, or 75 or more, or 76 or more, or 77 or more, or 78 or more, or 79 or more, 80 or more, or 81 or more, or 82 or more, or 83 or more, or 84 or more, or 85 or more, or 86 or more, or 87 or more, or 88 or more, or 89 or more, 90 or more, or 91 or more, or 92 or more, or 93 or more, or 94 or more, or 95 or more, or 96 or more, or 97 or more, or 98 or more, or 99 or more, or 100 or more, or 101 or more, or 102 or more, or 103 or more, or 104 or more, or 105 or more, or 106 or more, or 107 or more, or 108 or more, or 109 or more, or 110 or more, or 111 or more, or 112 or more, or 113 or more, or 114 or more, or 115 or more, or 116 or more, or 117 or more, or 118 or more, or 119 or more, or 120 or more, or 121 or more, or 122 or more, or 123 or more, or 124 or more, or 125 or more, or 126 or more, or 127 or more, or 128 or more, or 129 or more, or 130 or more, or 131 or more, or 132 or more, 133 or more, or 134 or more, or 135 or more, or 136 or more, or 137 or more, or 138 or more, or 139 or more, or 140 or more, or 141 or more, or 142 or more, or 143 or more, or 144 or more, or 145 or more, or 146 or more, or 147 or more, or 148 or more, or 149 or more, 150 or more, 151 or more, or 152 or more, or 153 or more, or 154 or more, or 155 or more, or 156 or more, or 157 or more, or 158 or more, or 159 or more, 160 or more, or 161 or more, or 162 or more, or 163 or more, or 164 or more, or 165 or more, or 166 or more, or 167 or more, or 168 or more, or 169 or more, 170 or more, or 171 or more, or 172 or more, or 173 or more, or 174 or more, or 175 or more, or 176 or more, or 177 or more, or 178 or more, or 179 or more, 180 or more, or 181 or more, or 182 or more, or 183 or more, or 184 or more, or 185 or more, or 186 or more, or 187 or more, or 188 or more, or 189 or more, 190 or more, or 191 or more, or 192 or more, or 193 or more, or 194 or more, or 195 or more, or 196 or more, or 197 or more, or 198 or more, or 199 or more, or 200 or more, or 201 or more, or 202 or more, or 203 or more, or 204 or more, or 205 or more, or 206 or more, or 207 or more, or 208 or more, or 209 or more, or 210 or more, or 211 or more, or 212 or more, or 213 or more, or 214 or more, or 215 or more, or 216 or more, or 217 or more, or 218 or more, or 219 or more, or 220 or more, or 221 or more, or 222 or more, or 223 or more, or 224 or more, or 225 or more, or 226 or more, or 227 or more, or 228 or more, or 229 or more, or 230 or more, or 231 or more, or 232 or more, or 233 or more, or 234 or more, or 235 or more, or 236 or more, or 237 or more, or 238 or more, or 239 or more, or 240 or more, or 241 or more, or 242 or more, or 243 or more, or 244 or more, or 245 or more, or 246 or more, or 247 or more, or 248 or more, or 249 or more, 250 or more, 251 or more, or 252 or more, or 253 or more, or 254 or more, or 255 or more, or 256 or more, or 257 or more, or 258 or more, or 259 or more, 260 or more, or 261 or more, or 262 or more, or 263 or more, or 264 or more, or 265 or more, or 266 or more, or 267 or more, or 268 or more, or 269 or more, 270 or more, or 271 or more, or 272 or more genes selected from Table 1b.

In embodiments, one or more genes selected from Table 1b comprises at least one of RAP1GAP, E2F2, RSRP1, RHD, RHCE, ERMAP, SLC2A1, CD58, SELENBP1, PPOX, NPL, ADIPOR1, BTG2, KLHDC8A, SDE2, GUK1, LBH, LTBP1, ZC3H6, TRAK2, STRADB, TMBIM1, DNAJB2, KAT2B, ABHD5, CPOX, RAB6B, PAQR9, SIAH2, NCEH1, KLF3, FRYL, MOB1B, HERC6, TSPAN5, GYPE, GYPB, FHDC1, CLCN3, ANKH, EPB41L4A, IRF1, CYSTM1, FAXDC2, TRIM10, TSPO2, CCND3, GTPBP2, GCLC, FOXO3, SERINC1, CITED2, JAZF1, MTURN, CD36, PNPLA8, BPGM, KDM7A, MFHAS1, CTSB, SLC25A37, BNIP3L, RNF19A, GRINA, HSF1, AQP3, CTSL, TMOD1, STOM, RXRA, OPTN, FRMD4A, STAM, MXI1, UROS, RIC8A, ILK, SOX6, CAT, YPEL4, UCP2, PPME1, ENDOD1, PTMS, CMAS, NFE2, KIF5A, RAB3IP, NUDT4, HECTD4, SDSL, RB1, PNP, DCAF11, ATP6V1D, DPF3, ZFYVE21, KIF26A, KLF13, CCNDBP1, EPB42, REXO5, ITFG1, TERF2IP, SLC7A5, EPN2,NATD1, PLEKHH3, SLC4A1, FAM117A, WIPI1, SMIM5, LPIN2, RIOK3, UBXN6, 2-Mar, JUNB, AKAP8L, UPK1A-AS1, PPP1R15A, GPCPD1, FAM210B, RBM38, TUBB1, ITSN1, GRAP2, WWC3, ALAS2, FAM122C, MOSPD1, SOX4, CLSTN1, PGM1, CD2, PHGDH, MLLT11, IFI16, FCER1A, RGS18, CD34, NENF, PLEK, IL1B, IGFBP7, INPP4B, BASP1, FYB1, CD74, SERPINB6, TUBB2B, LTB, LST1, AIF1, MPIG6B, HLA-DRA, HLA-DRB1, HLA-DMA, HLA-DPA1, HLA-DPB1, MAP7, CPVL, SCRN1, GNAI1, CYP3A4, PRKAG2, CLU, PVT1, ALDH1A1, NFIL3, DNM1, FAM69B, NPDC1, VIM, ARID5B, ZMIZ1, SESN2, GADD45A, DENND2D, FAM91A3P, S100A6, IER5, RGS16, PHLDA3, XPC, LXN, ZMAT3, CDKN1A, SESN1, PLIN2, RPS6, CDKN2A, ANKRD18A, LCN12, FAS, CTSD, HBBP1, DDB2, CTTN, SNORD15B, MDM2, PXMP2, PLEK2, RPS27L, LYRM1, RNF167, RNU4-34P, MYL4, FDXR, TNFSF9, CD70, GDF15, ECH1, BBC3, BAX, FTL, RPS5, ADA, GNAS, APOBEC3H, RHOC, CYP1B1, SUCNR1, TIPARP, HSD17B11, HLA-A, HLA-B, PSMB9, ASAH1, VPS28, HACD1, BGLT3, HBG1, HBG2, TRIM22, PRDX5, TSC22D1, RGS6, IFI27L2, B2M, ARID3A, RABAC1, and BEX1.

In embodiments, contacting the population of progenitor cells occurs in vitro or ex vivo or in vivo in a subject.

In aspects and embodiments, there is provided a perturbagen for use in any of the disclosed methods.

In aspects and embodiments, there is provided a pharmaceutical composition comprising a perturbagen of the disclosure.

In aspects and embodiments, there is provided a method for treating a disease or disorder characterized by an abnormal oxygen delivery, comprising, (a) administering to a subject in need thereof a therapeutically effective amount of at least one perturbagen selected from Table 3, or a variant thereof, or (b) administering to a subject in need thereof a cell, the cell having been contacted with at least one perturbagen selected from Table 3, or a variant thereof.

In aspects and embodiments, there is provided a method for treating a disease or disorder characterized by a hemoglobin deficiency, comprising, (a) administering to a subject in need thereof a therapeutically effective amount of at least one perturbagen selected from Table 3, or a variant thereof, or (b) administering to a subject in need thereof a cell, the cell having been contacted with at least one perturbagen selected from Table 3, or a variant thereof.

In aspects and embodiments, there is provided a method for treating or preventing a sickle cell disease or a thalassemia, comprising, (a) administering to a subject in need thereof a therapeutically effective amount of at least one perturbagen selected from Table 3, or a variant thereof, or (b) administering to a subject in need thereof a cell, the cell having been contacted with at least one perturbagen selected from Table 3, or a variant thereof.

In aspects and embodiments, there is provided a method for treating a disease or disorder characterized by an abnormal oxygen delivery, comprising, (a) administering to a subject in need thereof a therapeutically effective amount of at least one perturbagen selected from Table 3, or a variant thereof, wherein the at least one perturbagen is capable of changing a gene signature in a progenitor cell or (b) administering to a subject in need thereof a cell, the cell having been contacted with at least one perturbagen selected from Table 3, or a variant thereof, wherein the at least one perturbagen is capable of changing a gene signature in a progenitor cell.

In aspects and embodiments, there is provided a method for treating a disease or disorder characterized by a hemoglobin deficiency, comprising, (a) administering to a subject in need thereof a therapeutically effective amount of at least one perturbagen selected from Table 3, or a variant thereof, wherein the at least one perturbagen is capable of changing a gene signature in a progenitor cell or (b) administering to a subject in need thereof a cell, the cell having been contacted with at least one perturbagen selected from Table 3, or a variant thereof, wherein the at least one perturbagen is capable of changing a gene signature in a progenitor cell.

In aspects and embodiments, there is provided a method for treating or preventing a sickle cell disease or a thalassemia, comprising, (a) administering to a subject in need thereof a therapeutically effective amount of at least one perturbagen selected from Table 3, or a variant thereof, wherein the at least one perturbagen is capable of changing a gene signature in a progenitor cell or (b) administering to a subject in need thereof a cell, the cell having been contacted with at least one perturbagen selected from Table 3, or a variant thereof, wherein the at least one perturbagen is capable of changing a gene signature in a progenitor cell.

In embodiments, the disease or disorder characterized by an abnormal oxygen delivery and/or a hemoglobin deficiency is an anemia.

In embodiments, the sickle cell disease or a thalassemia is beta-thalassemia (transfusion dependent), or beta-thalassemia major, or beta-thalassemia intermedia, or beta-thalassemia minor, or wherein the sickle cell disease or a thalassemia is sickle cell anemia (SS), sickle hemoglobin-C disease (SC), sickle beta-plus thalassemia and sickle beta-zero thalassemia. In embodiments, at least one perturbagen is capable of changing a gene signature in a progenitor cell.

In embodiments, the at least one perturbagen is capable of changing a gene signature in a progenitor cell.

In embodiments, the subject is selected by steps comprising, obtaining from the subject having the disease or disorder a sample of cells comprising a non-lineage committed CD34+ cell; and contacting the sample of cells with least one perturbagen selected from Table 3, or a variant thereof.

In embodiments, the subject is selected by steps comprising, obtaining from the subject having the disease or disorder a sample of cells comprising a non-lineage committed CD34+ cell; and contacting the sample of cells with least one perturbagen selected from Table 3, or a variant thereof, wherein the at least one perturbagen alters a gene signature in the sample of cells.

In embodiments, the subject is selected by steps comprising, obtaining from a subject having the disease or disorder a sample of cells comprising a non-lineage committed CD34+ cell; and contacting the sample of cells with at least one perturbagen capable of altering a gene signature in a non-lineage committed CD34+ cell, wherein the at least one perturbagen increases in the sample of cells the expression and/or activity of one or more genes selected from the genes designated as an “up” gene in the gene directionality column of Table 1a and/or Table 2 and/or decreases in the sample of cells the expression and/or activity of one or more genes selected from the genes designated as a “down” gene in the gene directionality column of Table 1a and/or Table 2 and/or increases in the sample of cells the expression and/or activity of one or more genes selected from Table 1b and/or decreases in the sample of cells the expression and/or activity of one or more genes selected from Table 1b.

In embodiments, the subject is selected by steps comprising, obtaining from a subject having the disease or disorder a sample of cells comprising a non-lineage committed CD34+ cell; and contacting the sample of cells with at least one perturbagen selected from Table 3, or a variant thereof, wherein the at least one perturbagen increases in the sample of cells the expression and/or activity of one or more genes selected from the genes designated as an “up” gene in the gene directionality column of Table 1a and/or Table 2 and/or decreases in the sample of cells the expression and/or activity of one or more genes selected from the genes designated as a “down” gene in the gene directionality column of Table 1a and/or Table 2 and/or increases in the sample of cells the expression and/or activity of one or more genes selected from Table 1b and/or decreases in the sample of cells the expression and/or activity of one or more genes selected from Table 1b.

In aspects and embodiments, there is provided a use of the perturbagen of Table 3, or a variant thereof in the manufacture of a medicament for treating a disease or disorder characterized an abnormal oxygen delivery or a hemoglobin deficiency.

In aspects and embodiments, there is provided a use of the perturbagen of Table 3 or a variant thereof in the manufacture of a medicament for treating sickle cell disease or a thalassemia.

In aspects and embodiments, there is provided a method of identifying a candidate perturbation for promoting the transition of a starting population of progenitor cells into erythrocytes comprising HbF or HbF-expressing progenitors thereof, the method comprising, exposing the starting population of progenitor cells to a perturbation, identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into erythrocytes comprising HbF or HbF-expressing progenitors thereof following exposure of the population of cells to the perturbation, and identifying the perturbation as a candidate perturbation for promoting the transition of a population of progenitor cells into erythrocytes comprising HbF or HbF-expressing progenitors thereof based on the perturbation signature, wherein the perturbation signature is an increase in expression and/or activity in the progenitor cell of one or more genes selected from the genes designated as an “up” gene in the gene directionality column of Table 1a and/or Table 2, and/or a decrease in expression and/or activity in the progenitor cell of one or more genes selected from the genes designated as a “down” gene in the gene directionality column of Table 1a and/or Table 2 and/or an increase in expression and/or activity in the progenitor cell of one or more genes selected from Table 1b and/or a decrease in expression and/or activity in the progenitor cell of one or more genes selected from Table 1b.

In embodiments, the perturbation signature is an increase in expression and/or activity in the progenitor cell of a network module designated in the network module column of Table 1a and/or Table 1b.

In aspects and embodiments, there is provided a method for making a therapeutic agent for a disease or disorder selected from a sickle cell disease or a thalassemia or a disease or disorder characterized by an abnormal oxygen delivery or a hemoglobin deficiency, comprising, (a) identifying a candidate perturbation for therapy according to a method of the disclosure and (b) formulating the candidate perturbation as a therapeutic agent for the treatment of the disease or disorder.

In embodiments, at least one perturbagen is administered in combination with a therapeutically effective amount of one or more additional therapeutic agents. In embodiments, the additional therapeutic agent is hydroxyurea (HU).

In aspects and embodiments, there is provided a method for directing a change in cell state of a progenitor cell, comprising: contacting a population of cells comprising a progenitor cell with at least one perturbagen capable of altering a gene signature in the progenitor cell, wherein altering the gene signature comprises an increase in expression and/or activity in the progenitor cell of one or more genes selected from the genes designated as an “up” gene in the gene directionality column of Table 1a and/or Table 2 and/or a decrease in expression and/or activity in the progenitor cell of one or more genes selected from the genes designated as a “down” gene in the gene directionality column of Table 1a and/or Table 2 and/or an increase in expression and/or activity in the progenitor cell of one or more genes selected from Table 1b and/or a decrease in expression and/or activity in the progenitor cell of one or more genes selected from Table 1b; wherein the progenitor cell is a non-lineage committed CD34+ cell; the change in cell state provides an increase in the number of one or more of proerythroblasts, early erythroblasts, intermediate erythroblasts, late erythroblasts, reticulocytes, and erythrocytes, and the increase in the number of proerythroblasts, early erythroblasts, intermediate erythroblasts, late erythroblasts, reticulocytes, and/or erythrocytes is relative to the number of proerythroblasts, early erythroblasts, intermediate erythroblasts, late erythroblasts, reticulocytes, and/or erythrocytes obtained from a population of progenitor cells that is not contacted with the at least one perturbagen.

In embodiments, the at least one perturbagen is selected from Table 3, or a variant thereof.

In embodiments, the at least one perturbagen selected from Table 3, or a variant thereof, comprises at least 2, at least 3, at least 4, or at least 5 perturbagens selected from Table 3, or variants thereof.

In embodiments, one or more of the proerythroblasts, early erythroblasts, intermediate erythroblasts, late erythroblasts, reticulocytes, and erythrocytes expresses fetal hemoglobin (HbF) expresses HBG1 and/or HBG2.

In embodiments, one or more of the proerythroblasts, early erythroblasts, intermediate erythroblasts, late erythroblasts, reticulocytes, and erythrocytes expresses fetal hemoglobin (HbF).

In embodiments, the at least one perturbagen is used in combination with one or more additional therapeutic agents.

In embodiments, the additional therapeutic agent is hydroxyurea (HU).

In embodiments, the one or more genes selected from the genes designated as an “up” gene in the gene directionality column of Table 1a comprises at least one of DDIT4, EPRS, MTHFD2, EIF4EBP1, AARS, ABCC5, PHGDH, TUBB6, LSM6, EIF4G1, RNF167, CD320, CTNNAL1, GADD45A, PTK2, CFLAR, IGF2BP2, CDK1, CDC45, CDCA4, MELK, HAT1, PAK1, TSPAN6, TIMM17B, KDM5A, UBE3B, RPS5, PAICS, RPIA, KDELR2, PNP, CAST, H2AFV, ATP11B, CTNND1, ORC1, FDFT1, CDKN1B, INSIG1, IGF1R, TRAP1, TSTA3, SUZ12, CDK4, HMGCS1, LAP3, TBPL1, FAH, CCP110, APOE, IGF2R, DYRK3, MYBL2, APP, DNMT1, SMC3, HTATSF1, CAT, ACAT2, HK1, PSMD4, CLTC, MAP4K4, PROS1, DLD, SDHB, GNAS, COPS7A, MPC2, HEBP1, BLVRA, ID2, SCAND1, ETFB, MRPS16, PIN1, TRAK2, AMDHD2, PLEKHJ1, BZW2, PCNA, WDR61, RFC5, OXA1L, MCM3, CEP57, PSMF1, POLR2K, PSMD2, ATP6V1D, PSMD9, AKAP8L, GRN, SPAG7, ENOSF1, PCK2, PCCB, NOLC1, EBNA1BP2, CD58, RFC2, ASAH1, LAGE3, AKR7A2, and RSU1.

In embodiments, the one or more genes selected from the genes designated as a “down” gene in the gene directionality column of Table 1a comprises at least one of NUCB2, XBP1, CCNB1, CDC20, PLK1, CDK6, ITGB1 BP1, CCNE2, PTPN6, CBR1, HLA-DRA, MAP7, SOX4, CASP3, DNAJB6, HOXA10, IL1B, ICAM3, ADGRG1, HLA-DMA, PDLIM1, PSMB8, EPB41L2, RPL39L, PYGL, CYB561, and HOMER2.

In embodiments, the one or more genes selected from the genes designated as an “up” gene in the gene directionality column of Table 2 comprises at least one of HMGA1, KLF1, KLF6, SREBF1, NFE2, ARID3A, GFI1B, KLF13, MLXIP, E2F8, MYBL2, HSF1, GMEB1, NFX1, TGIF1, KLF3, SP1, CENPX, HES6, and LIN28B.

In embodiments, the one or more genes selected from the genes designated as a “down” gene in the gene directionality column of Table 2 comprises at least one of HOXA10, XBP1, SOX4, ZNF385D, NFIC, BATF, HHEX, RARG, KDM5B, ZFX, SPI1, TEAD4, SATB1, NFIX, PLAGL1, MEF2C, ZBTB1, HOXA9, THAP5, and ZFP57.

In embodiments, the one or more genes selected from Table 1b comprises at least one of RAP1GAP, E2F2, RSRP1, RHD, RHCE, ERMAP, SLC2A1, CD58, SELENBP1, PPOX, NPL, ADIPOR1, BTG2, KLHDC8A, SDE2, GUK1, LBH, LTBP1, ZC3H6, TRAK2, STRADB, TMBIM1, DNAJB2, KAT2B, ABHD5, CPOX, RAB6B, PAQR9, SIAH2, NCEH1, KLF3, FRYL, MOB1B, HERC6, TSPAN5, GYPE, GYPB, FHDC1, CLCN3, ANKH, EPB41L4A, IRF1, CYSTM1, FAXDC2, TRIM10, TSPO2, CCND3, GTPBP2, GCLC, FOXO3, SERINC1, CITED2, JAZF1, MTURN, CD36, PNPLA8, BPGM, KDM7A, MFHAS1, CTSB, SLC25A37, BNIP3L, RNF19A, GRINA, HSF1, AQP3, CTSL, TMOD1, STOM, RXRA, OPTN, FRMD4A, STAM, MXI1, UROS, RIC8A, ILK, SOX6, CAT, YPEL4, UCP2, PPME1, ENDOD1, PTMS, CMAS, NFE2, KIF5A, RAB3IP, NUDT4, HECTD4, SDSL, RB1, PNP, DCAF11, ATP6V1D, DPF3, ZFYVE21, KIF26A, KLF13, CCNDBP1, EPB42, REXO5, ITFG1, TERF2IP, SLC7A5, EPN2,NATD1, PLEKHH3, SLC4A1, FAM117A, WIPI1, SMIM5, LPIN2, RIOK3, UBXN6, 2-Mar, JUNB, AKAP8L, UPK1A-AS1, PPP1R15A, GPCPD1, FAM210B, RBM38, TUBB1, ITSN1, GRAP2, WWC3, ALAS2, FAM122C, MOSPD1, SOX4, CLSTN1, PGM1, CD2, PHGDH, MLLT11, IFI16, FCER1A, RGS18, CD34, NENF, PLEK, IL1B, IGFBP7, INPP4B, BASP1, FYB1, CD74, SERPINB6, TUBB2B, LTB, LST1, AIF1, MPIG6B, HLA-DRA, HLA-DRB1, HLA-DMA, HLA-DPA1, HLA-DPB1, MAP7, CPVL, SCRN1, GNAI1, CYP3A4, PRKAG2, CLU, PVT1, ALDH1A1, NFIL3, DNM1, FAM69B, NPDC1, VIM, ARID5B, ZMIZ1, SESN2, GADD45A, DENND2D, FAM91A3P, S100A6, IER5, RGS16, PHLDA3, XPC, LXN, ZMAT3, CDKN1A, SESN1, PLIN2, RPS6, CDKN2A, ANKRD18A, LCN12, FAS, CTSD, HBBP1, DDB2, CTTN, SNORD15B, MDM2, PXMP2, PLEK2, RPS27L, LYRM1, RNF167, RNU4-34P, MYL4, FDXR, TNFSF9, CD70, GDF15, ECH1, BBC3, BAX, FTL, RPS5, ADA, GNAS, APOBEC3H, RHOC, CYP1B1, SUCNR1, TIPARP, HSD17B11, HLA-A, HLA-B, PSMB9, ASAH1, VPS28, HACD1, BGLT3, HBG1, HBG2, TRIM22, PRDX5, TSC22D1, RGS6, IFI27L2, B2M, ARID3A, RABAC1, and BEX1.

### BRIEF DESCRIPTION OF THE DRAWINGS

FIG. 1A-FIG. 1C show images of cord blood/mPB Manifold with high fetal trajectories and annotations. FIG. 1A shows that proxies for cellular states are the annotated clusters. FIG. 1B shows that a pseudo-trajectory associated with a fetal erythropoiesis program marked by high expression of HBG1. FIG. 1C shows differential signatures for the 3 to 15 transition cell state.

FIG. 2 shows a representative of maturation profile of vehicle and Perturbagen 8 at day 14.

FIG. 3A and FIG. 3B show graphs of experimental data demonstrating % F-Cell at Day 14 of CD34+ Treated with Predicted Perturbagens Demonstrating an Increase in F-cell over vehicle control. In FIG. 3A, the Data bars are from left to right: Vehicle control; HU50 in DMSO; Perturbagen 1 (0.01 μM); Perturbagen 1 (0.03 μM); Perturbagen 1 (0.1 μM); Perturbagen 1 (0.3 μM); Perturbagen 2 (0.1 μM); Perturbagen 2 (1 μM); Perturbagen 2 (10 μM); Perturbagen 3 (0.1 μM); Perturbagen 3 (1 mM); Perturbagen 4 (0.1 μM); Perturbagen 4 (1 μM); Perturbagen 5 (0.1 μM); Perturbagen 5 (1 μM); Perturbagen 5 (10 μM); Perturbagen 6 (0.1 μM); Perturbagen 6 (1 μM); Perturbagen 7 (0.1 μM); Perturbagen 7 (1 μM); Perturbagen 7 (10 μM); Perturbagen 8 (0.0005 μM); Perturbagen 8 (0.001 μM); Perturbagen 8 (0.002 μM); Perturbagen 8 (0.002 μM); Perturbagen 8 (0.1 μM); Perturbagen 8 (0.1 μM); Perturbagen 9 (0.1 μM); Perturbagen 10 (0.1 μM); Perturbagen 10 (1 μM); Perturbagen 10 (10 μM); Perturbagen 11 (0.1 μM); Perturbagen 11 (1 mM); Perturbagen 12 (0.03 μM); Perturbagen 12 (0.1 μM); Perturbagen 13 (0.1 μM); Perturbagen 13 (1 μM); Perturbagen 13 (10 μM); Perturbagen 14 (0.1 μM); Perturbagen 14 (1 μM); Perturbagen 14 (10 μM); Perturbagen 15 (0.03 μM); Perturbagen 15 (0.1 μM); Perturbagen 16 (0.1 μM); Perturbagen 16 (10 μM); Perturbagen 17 (0.1 μM); Perturbagen 17 (1 μM); Perturbagen 18 (0.03 μM); Perturbagen 18 (0.1 μM); Perturbagen 19 (0.1 μM); Perturbagen 19 (1 μM); Perturbagen 20 (0.1 nM); Perturbagen 20 (1 nM); Perturbagen 20 (10 nM); Perturbagen 21 (0.1 μM); Perturbagen 21 (1 μM); Perturbagen 22 (0.1 μM); Perturbagen 22 (1 mM); Perturbagen 22 (10 μM); Perturbagen 23 (0.1 μM); Perturbagen 23 (1 μM); Perturbagen 23 (10 μM); Perturbagen 24 (0.1 μM); Perturbagen 24 (1 μM); Perturbagen 24 (10 μM); Perturbagen 25 (0.1 μM); Perturbagen 25 (1 μM); Perturbagen 25 (10 μM); Perturbagen 26 (0.03 μM); Perturbagen 26 (0.1 μM); Perturbagen 27 (0.1 μM); Perturbagen 27 (1 μM); Perturbagen 27 (10 μM); Perturbagen 28 (0.1 μM); Perturbagen 28 (1 μM); Perturbagen 28 (10 μM); Perturbagen 29 (0.1 μM); Perturbagen 29 (1 μM); Perturbagen 29 (10 μM); Perturbagen 30 (0.5 nM); Perturbagen 30 (5 nM); Perturbagen 30 (50 nM); Perturbagen 30 (0.1 μM); Perturbagen 31 (0.00003 μM); Perturbagen 31 (0.0003 μM); Perturbagen 31 (0.1 nM); Perturbagen 32 (10 μM); Perturbagen 32 (1 μM); Perturbagen 32 (0.1 μM); Perturbagen 33 (10 nM); Perturbagen 33 (1 nM); Perturbagen 33 (0.1 nM); Perturbagen 34 (0.0003 μM); Perturbagen 34 (0.1 nM); Perturbagen 35 (1 nM); Perturbagen 35 (0.1 nM); Perturbagen 35 (0.1 μM); Perturbagen 36 (0.1 μM); Perturbagen 36 (1 μM); Perturbagen 36 (5 μM); Perturbagen 37 (0.0005 μM); Perturbagen 37 (0.001 μM); Perturbagen 37 (0.005 μM); Perturbagen 37 (0.01 μM); Perturbagen 38 (0.01 μM); Perturbagen 38 (0.1 μM); Perturbagen 38 (1 μM); Perturbagen 39 (0.001 μM); Perturbagen 39 (0.01 μM); Perturbagen 40 (0.01 μM); Perturbagen 40 (0.1 μM); Perturbagen 40 (1 μM); Perturbagen 41 (0.03 μM); Perturbagen 41 (0.1 μM); Perturbagen 42 (0.000001 μM); and Perturbagen 42 (0.00001 μM). In FIG. 3B, the Data bars are from left to right: Vehicle control; HU50 in DMSO; Perturbagen 1 (0.01 μM); Perturbagen 2 (0.1 μM); Perturbagen 5 (10 μM); Perturbagen 7 (1 μM); Perturbagen 8 (0.1 μM); Perturbagen 9 (0.1 μM); Perturbagen 10 (10 μM); Perturbagen 11 (0.1 μM); Perturbagen 12 (0.03 μM); Perturbagen 13 (1 μM); Perturbagen 14 (10 μM); Perturbagen 15 (0.1 μM); Perturbagen 16 (0.1 μM); Perturbagen 17 (0.1 μM); Perturbagen 18 (0.03 μM); Perturbagen 19 (0.1 μM); Perturbagen 19 (1 μM); Perturbagen 20 (1 nM); Perturbagen 21 (0.1 μM); Perturbagen 21 (1 μM); Perturbagen 22 (1 mM); Perturbagen 23 (0.1 μM); Perturbagen 24 (1 μM); Perturbagen 25 (10 μM); Perturbagen 26 (0.03 μM); Perturbagen 27 (10 μM); Perturbagen 28 (1 μM); Perturbagen 29 (10 μM); Perturbagen 30 (50 nM); Perturbagen 31 (0.1 nM); Perturbagen 32 (1 μM); Perturbagen 33 (1 nM); Perturbagen 34 (0.0003 μM); Perturbagen 35 (1 nM); Perturbagen 36 (5 μM); Perturbagen 37 (0.01 μM); Perturbagen 38 (0.1 μM); Perturbagen 39 (0.001 μM); Perturbagen 40 (0.1 μM); Perturbagen 41 (0.03 μM); and Perturbagen 42 (0.00001 μM).

FIG. 4A and FIG. 4B show graphs of HPLC data demonstrating % HbF measured by HPLC at Day 14 of CD34+ Treated with Predicted Perturbagens Demonstrating significant increase above vehicle. In FIG. 4A, the Data bars are from left to right: DMSO Vehicle; Hydroxyurea in DMSO; Perturbagen 1 (0.01 μM); Perturbagen 1 (0.03 μM); Perturbagen 1 (0.1 μM); Perturbagen 2 (0.1 μM); Perturbagen 2 (1 μM); Perturbagen 2 (10 μM); Perturbagen 3 (0.1 μM); Perturbagen 3 (1 mM); Perturbagen 4 (0.1 μM); Perturbagen 4 (1 μM); Perturbagen 5 (0.1 μM); Perturbagen 5 (1 μM); Perturbagen 5 (10 μM); Perturbagen 6 (0.1 μM); Perturbagen 6 (1 μM); Perturbagen 7 (0.1 μM); Perturbagen 7 (1 μM); Perturbagen 7 (10 μM); Perturbagen 8 (0.0005 μM); Perturbagen 8 (0.001 μM); Perturbagen 8 (0.002 μM); Perturbagen 8 (0.002 μM); Perturbagen 8 (0.1 μM); Perturbagen 9 (0.0001 μM); Perturbagen 10 (0.1 μM); Perturbagen 10 (1 μM); Perturbagen 10 (10 μM); Perturbagen 11 (0.1 μM); Perturbagen 11 (1 mM); Perturbagen 12 (0.03 μM); Perturbagen 12 (0.1 μM); Perturbagen 13 (0.1 μM); Perturbagen 13 (1 μM); Perturbagen 13 (10 μM); Perturbagen 14 (0.1 μM); Perturbagen 14 (1 μM); Perturbagen 14 (10 μM); Perturbagen 15 (0.03 μM); Perturbagen 15 (0.1 μM); Perturbagen 16 (0.1 μM); Perturbagen 16 (10 μM); Perturbagen 17 (0.1 μM); Perturbagen 17 (1 μM); Perturbagen 18 (0.03 μM); Perturbagen 19 (0.1 μM); Perturbagen 19 (1 μM); Perturbagen 20 (0.1 nM); Perturbagen 20 (1 nM); Perturbagen 21 (0.1 μM); Perturbagen 21 (1 μM); Perturbagen 22 (0.1 μM); Perturbagen 22 (1 mM); Perturbagen 22 (10 μM); Perturbagen 23 (0.1 μM); Perturbagen 23 (1 μM); Perturbagen 23 (10 μM); Perturbagen 24 (0.1 μM); Perturbagen 24 (1 μM); Perturbagen 24 (10 μM); Perturbagen 25 (0.1 μM); Perturbagen 25 (1 μM); Perturbagen 25 (10 μM); Perturbagen 26 (0.03 μM); Perturbagen 26 (0.1 μM); Perturbagen 27 (0.1 μM); Perturbagen 27 (1 μM); Perturbagen 27 (10 μM); Perturbagen 28 (0.1 μM); Perturbagen 28 (1 μM); Perturbagen 28 (10 μM); Perturbagen 29 (0.1 μM); Perturbagen 29 (1 μM); Perturbagen 29 (10 μM); Perturbagen 30 (0.5 nM); Perturbagen 30 (5 nM); Perturbagen 30 (50 nM); Perturbagen 30 (0.1 μM); Perturbagen 31 (0.00003 μM); Perturbagen 31 (0.0003 μM); Perturbagen 31 (0.1 nM); Perturbagen 32 (0.1 μM); Perturbagen 32 (1 μM); Perturbagen 33 (0.1 nM); Perturbagen 33 (1 nM); Perturbagen 33 (10 nM); Perturbagen 34 (0.0003 μM); Perturbagen 34 (0.1 nM); Perturbagen 35 (0.1 nM); Perturbagen 35 (1 nM); Perturbagen 36 (0.1 μM); Perturbagen 36 (1 μM); Perturbagen 36 (5 μM); Perturbagen 37 (0.0005 μM); Perturbagen 37 (0.001 μM); Perturbagen 37 (0.005 μM); Perturbagen 38 (0.01 μM); Perturbagen 38 (0.1 μM); Perturbagen 38 (1 μM); Perturbagen 39 (0.001 μM); Perturbagen 39 (0.01 μM); Perturbagen 40 (0.01 μM); Perturbagen 40 (0.1 μM); Perturbagen 41 (0.03 μM); Perturbagen 41 (0.1 μM); Perturbagen 42 (0.000001 μM); and Perturbagen 42 (0.00001 μM). In FIG. 4B, the Data bars are from left to right: DMSO Vehicle; Hydroxyurea in DMSO; Perturbagen 1 (0.03 μM); Perturbagen 2 (0.1 μM); Perturbagen 5 (10 μM); Perturbagen 7 (1 μM); Perturbagen 8 (0.1 μM); Perturbagen 9 (0.0001 μM); Perturbagen 10 (10 μM); Perturbagen 11 (0.1 μM); Perturbagen 12 (0.03 μM); Perturbagen 13 (1 μM); Perturbagen 14 (10 μM); Perturbagen 15 (0.1 μM); Perturbagen 16 (0.1 μM); Perturbagen 17 (0.1 μM); Perturbagen 18 (0.03 μM); Perturbagen 19 (0.1 μM); Perturbagen 19 (1 μM); Perturbagen 20 (1 nM); Perturbagen 21 (0.1 μM); Perturbagen 21 (1 μM); Perturbagen 22 (1 mM); Perturbagen 23 (0.1 μM); Perturbagen 24 (0.1 μM); Perturbagen 24 (1 μM); Perturbagen 25 (10 μM); Perturbagen 26 (0.03 μM); Perturbagen 27 (10 μM); Perturbagen 28 (1 μM); Perturbagen 29 (10 μM); Perturbagen 30 (50 nM); Perturbagen 31 (0.1 nM); Perturbagen 32 (1 μM); Perturbagen 33 (1 nM); Perturbagen 34 (0.0003 μM); Perturbagen 35 (1 nM); Perturbagen 36 (5 μM); Perturbagen 37 (0.005 μM); Perturbagen 38 (0.01 μM); Perturbagen 39 (0.001 μM); Perturbagen 40 (0.1 μM); Perturbagen 41 (0.03 μM); and Perturbagen 42 (0.00001 μM).

FIG. 5 shows images of the manifold of the two HbF trajectories and annotations.

FIG. 6A shows images of flow cytometry of day 3 mPB CD34+ treated with HU demonstrating the emergence of a cell population defined by CD34+CD41lowCD235a+ expression. FIG. 6B shows a graph of experimental data demonstrating % F-cells of vehicle and HU treated samples at the end of erythroid differentiation.

FIG. 7 shows a validation/module of HU cell state based on scRNA sequencing.

FIG. 8 shows images of flow cytometry demonstrating an increase in CD34+CD41LowCD235a+ population with co-treatment with HU and Dexamethasone.

FIG. 9A and FIG. 9B show the synergy of HU and HU+Dex at increasing % F-cells.

FIG. 10 shows the validation of HU induction of NR3C1 from scRNA sequencing.

FIG. 11A and FIG. 11B show F-cell data demonstrating that a second glucocorticoid agonist results in synergy with HU. FIG. 11A shows absolute change. FIG. 11B shows fold change.

FIG. 12 shows percentage of globin synthesis in yolk sac, liver, spleen, and bone marrow from a gestational age of 0 months to infancy (see Sankaran V. et. al., Cold Spring Harb. Perspect. Med. 3:a011643 (2013), which is incorporated by reference herein in its entirety).

FIGS. 13A-13B shows the generation of high-resolution map of fetal and adult erythropoiesis. FIG. 13A shows a workflow schematic of a serum free human in vitro erythroid differentiation assay using scRNA sequencing of 6 donors at 8 time points. FIG. 13B shows a unified umap of transcriptionally distinct pseudo-trajectories and cell states associated with fetal hemoglobin induction marked by HBG1/HBB expression.

FIG. 14 shows the identification of gene signature associated with fetal erythropoiesis using a machine learning platform and the identification of targetable gene signature and small molecules to induce HbF.

FIGS. 15A-15G show experimental data demonstrating the identification of Perturbagen 1081 as an inducer of fetal hemoglobin. FIG. 15A shows a table demonstrating the validation of predicted compounds. Adult human CD34+ progenitors were exposed to predicted perturbagens targeting a fetal erythropoiesis gene signature and their ability to induce fetal hemoglobin was measured by % HbF (HPLC) and % F-cells (flow-cytometry). Dots represent average induction of each perturbation tested (n=3), identifying Perturbagen 1081 as strong as HbF inducer. FIG. 15B shows high performance liquid chromatography (HPLC) analysis of in vitro derived erythrocytes on Day 14. HPLC analysis demonstrated that Perturbagen 1081 induced HbF (42.3%+17.26, n=4) above HU (17.16%+4.78, n=5) and BCL11A CRISPR knockdown (32.58%+10.66, n=5) ANOVA followed by Dunnett's vs Control, *p<0.05, **p<0.01, ****p<0.00001. Concentration for HU is 50 μM and for Perturbagen 1081 is 0.1 μM. FIG. 15C shows a table of Perturbagen 1081 dose response. Exposure of CD34+ progenitors with Perturbagen 1081 demonstrate a dose dependent induction of HbF as measure by HPLC (single donor). FIGS. 15D-15G show temporal globin expression for Perturbagen 1081. Temporal gene expression profiling which revealed that Perturbagen 1081 induced robust induction of HBG1 and HBG2 and concomitant decrease in HBB expression (n=3, single donor).

FIGS. 16A-16C show experimental data demonstrating that Perturbagen 1081 induced predicted gene signature associated with fetal hemoglobin induction and validation of Perturbagen 1081 induced gene signature. FIG. 16A shows Perturbagen 1081 treatment and BLC11A-KD resulted in cell density shift towards the fetal trajectory. Adult human CD34+ progenitors were treated with Perturbagen 1081 and other known HbF inducers and subjected to scRNA-seq. Analysis revealed adult and fetal pseudo-trajectories marked by HBG2/HBB differential gene expression. Perturbagen 1081 treatment and BLC11A-KD resulted in cell density shift towards the fetal trajectory. FIG. 16B shows the transcriptional correlation of Perturbagen 1081 to BLC11A-KD. Comparison between cell responses to Perturbagen 1081 vs BCL11A-KD revealed significant transcriptional correlation (Pearson's, p-value=0.0011) between the two perturbations. FIG. 16C shows the targeted fetal signature. An analysis of Perturbagen 1081 induced gene signature validated the fetal erythropoiesis signature predicted using the machine learning platform.

### DETAILED DESCRIPTION

The present disclosure is based, in part, on the discovery that cells of hematopoietic lineages comprising erythroblasts, reticulocytes, and erythrocytes, and their progenitors can be characterized by specific gene signatures. Additionally, the present disclosure is based on the discovery that certain active agents (i.e., perturbagens) can alter these specific gene signatures. Such alteration is associated with the acquisition of specific cell states by cells of erythrocyte and/or erythroid lineages. These perturbagens are, in some instance, useful as therapeutics and derive benefit by directing the reactivation of fetal hemoglobin (HbF).

### Gene Signature

Cell state transitions (i.e., a transition in a cell's state from a first cell state to a second cell state, e.g., differentiation) are characterized by a change in expression of genes in the cell. Changes in gene expression may be quantified as, e.g., an increase in mRNA expressed for a specific gene or a decrease in mRNA expressed for another specific gene; especially significant here may be mRNAs that encode transcription factors. Collectively, the sum of multiple differences in gene expression between one cell type or cells of one lineage relative to another cell type or cells of another lineage are referred to herein as a gene signature.

Any one of a number of methods and metrics may be used to identify gene signatures. Non-limiting examples include single cell and bulk RNA sequencing with or without prior cell sorting (e.g., fluorescence activated cell sorting (FACS) and flow cytometry). When developing a gene signature, it may useful to first characterize the cell type or cells of a specific lineage by surface proteins (i.e., antigen expression) that are characteristic of the cell type or cells of a specific lineage.

The erythroid progenitor cells at different maturation stages may be characterized by its antigen expression. The erythroblasts express transferrin receptor (also known as CD71 in human) and glycophorin A (GlyA, also known as CD235a in human) (Hattangadiet. al., Blood, 2011, 118 (24):6258-68.), but express little or no hemoglobin (Hb). The erythroblasts have the capacity to mature into hemoglobinized erythrocytes and reticulocytes. During maturation, CD71 expression decreases but remains detectable on most cells, GlyA expression remains high or increases further, and cell pellets become visibly red due to the accumulation of Hb.

Genome-wide association studies (GWAS) demonstrated that about half of the heritable variation in HbF level is due to polymorphism in three loci including β-globin cluster, an intergenic interval between the HBS1L and MYB genes, and the BCL11A gene. BCL11A gene is a zinc-finger transcriptional factor that functions as a developmental stage-specific repressor of HbF expression. (Sankaran et al. Human fetal hemoglobin expression is regulated by the developmental stage-specific repressor BCL11A, Science. 2008; 322:1839-1842; Sankaran et al. Developmental and species-divergent globin switching are driven by BCL11A, Nature. 2009; 460:1093-10). KLF1 gene is reported as a DNA-binding transcription factor that activates BCL11A expression by associating with the BCL11A promoter, suggesting a dual role of KLF1 gene in globin gene regulation by both functioning as a direct activator of adult-stage β-globin and indirect repressor of fetal-stage γ-globin (Zhou et al. KLF1 regulates BCL11A expression and γ- to β-globin gene switching, Nat Genet, 2010; vol. 42, pp. 742-744).

Fetal hemoglobin also known as hemoglobin F, HbF, or α2γ2) is as a hemoglobin tetramer composed of gamma globin subunit encoded by the HBG1 or HBG2 gene. Fetal hemoglobin is the main oxygen transport protein in the human fetus during the last seven months of development in the uterus and persists in the newborn until roughly 2-4 months old. Fetal hemoglobin differs most from adult hemoglobin in that it is able to bind oxygen with greater affinity than the adult form the (the p₅₀ of fetal hemoglobin is roughly 19 mmHg, whereas adult hemoglobin is approximately 26.8 mmHg). Like most types of normal hemoglobin, fetal hemoglobin is a tetramer composed of four protein subunits and four heme prosthetic groups. However, adult hemoglobin is composed of two α (alpha) and two β (beta) subunits, while fetal hemoglobin is composed of two a subunits and two γ (gamma) subunits (α2γ2).

Knowing the gene signature for each cell type or cells of a specific lineage provides insight into what genes impact or are associated with the process of transition to other cell types and/or differentiation of progenitor cells.

Gene signatures can be used to identify particular cells as being on-lineage, and other cells as being “progenitor” cells or intermediate cells along a transition trajectory towards the on-lineage cell type.

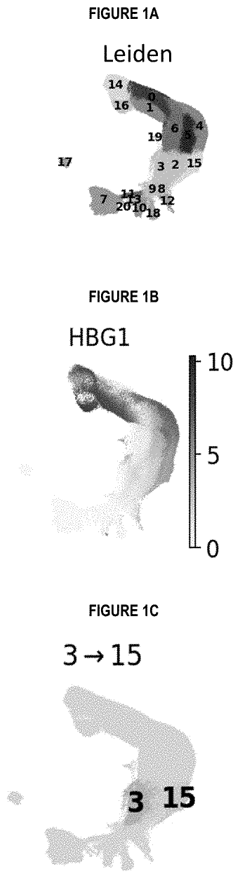

FIG. 1C shows annotated clusters that associate gene signature with cell types or cells of a specific lineage. Differential gene signatures for the 3 to 15 transition, i.e., from a non-lineage committed CD34⁺ progenitor cell to cells of the erythroid lineage expressing HBG1, were used to predict perturbations that would promote the transition.

Genes that are differentially expressed and positively associated with the promotion of erythroid lineage progression and/or erythrocyte differentiation are listed in Table 1a, Table 1b, and Table 2.

TABLE 1a
Genes showing a change in expression.

| | | | Gene_Di- | |
|---|---|---|---|---|
| | Gene | Gene_Entrez_ID | rectionality | Network_Module |
| 0 | DDIT4 | 54541 | up | 0 |
| 1 | EPRS | 2058 | up | 0 |
| 2 | MTHFD2 | 10797 | up | 0 |
| 3 | EIF4EBP1 | 1978 | up | 0 |
| 4 | AARS | 16 | up | 0 |
| 5 | ABCC5 | 10057 | up | 0 |
| 6 | PHGDH | 26227 | up | 0 |
| 7 | NUCB2 | 4925 | down | 0 |
| 8 | XBP1 | 7494 | down | 0 |
| 9 | TUBB6 | 84617 | up | 1 |
| 10 | LSM6 | 11157 | up | 1 |
| 11 | EIF4G1 | 1981 | up | 1 |
| 12 | RNF167 | 26001 | up | 1 |
| 13 | CD320 | 51293 | up | 1 |
| 14 | CCNB1 | 891 | down | 1 |
| 15 | CDC20 | 991 | down | 1 |
| 16 | PLK1 | 5347 | down | 1 |
| 17 | CTNNAL1 | 8727 | up | 2 |
| 18 | GADD45A | 1647 | up | 2 |
| 19 | PTK2 | 5747 | up | 2 |
| 20 | CFLAR | 8837 | up | 2 |
| 21 | IGF2BP2 | 10644 | up | 2 |
| 22 | CDK6 | 1021 | down | 2 |
| 23 | ITGB1BP1 | 9270 | down | 2 |
| 24 | CDK1 | 983 | up | 3 |
| 25 | CDC45 | 8318 | up | 3 |
| 26 | CDCA4 | 55038 | up | 3 |
| 27 | MELK | 9833 | up | 3 |
| 28 | HAT1 | 8520 | up | 3 |
| 29 | CCNE2 | 9134 | down | 3 |
| 30 | PAK1 | 5585 | up | 4 |
| 31 | TSPAN6 | 7105 | up | 4 |
| 32 | TIMM17B | 10245 | up | 4 |
| 33 | KDM5A | 5927 | up | 4 |
| 34 | UBE3B | 89910 | up | 4 |
| 35 | PTPN6 | 5777 | down | 4 |
| 36 | RPS5 | 6193 | up | 5 |
| 37 | PAICS | 10606 | up | 5 |
| 38 | RPIA | 22934 | up | 5 |
| 39 | KDELR2 | 11014 | up | 5 |
| 40 | PNP | 4860 | up | 5 |
| 41 | CBR1 | 873 | down | 5 |
| 42 | CAST | 10849 | up | 6 |
| 43 | H2AFV | 94239 | up | 6 |
| 44 | ATP11B | 23200 | up | 6 |
| 45 | CTNND1 | 1500 | up | 6 |
| 46 | ORC1 | 4998 | up | 6 |
| 47 | HLA-DRA | 3122 | down | 6 |
| 48 | FDFT1 | 2222 | up | 7 |
| 49 | CDKN1B | 1027 | up | 7 |
| 50 | INSIG1 | 3638 | up | 7 |
| 51 | IGF1R | 3480 | up | 7 |
| 52 | MAP7 | 9053 | down | 7 |
| 53 | TRAP1 | 7190 | up | 8 |
| 54 | TSTA3 | 7264 | up | 8 |
| 55 | SUZ12 | 23512 | up | 8 |
| 56 | CDK4 | 1019 | up | 8 |
| 57 | SOX4 | 6659 | down | 8 |
| 58 | HMGCS1 | 3157 | up | 9 |
| 59 | LAP3 | 51056 | up | 9 |
| 60 | TBPL1 | 9519 | up | 9 |
| 61 | FAH | 2184 | up | 9 |
| 62 | CCP110 | 9738 | up | 9 |
| 63 | APOE | 348 | up | 10 |
| 64 | IGF2R | 3482 | up | 10 |
| 65 | DYRK3 | 8444 | up | 10 |
| 66 | CASP3 | 836 | down | 10 |
| 67 | DNAJB6 | 10049 | down | 10 |
| 68 | MYBL2 | 4605 | up | 11 |
| 69 | APP | 351 | up | 11 |
| 70 | DNMT1 | 1786 | up | 11 |
| 71 | SMC3 | 9126 | up | 11 |
| 72 | HTATSF1 | 27336 | up | 11 |
| 73 | CAT | 10249 | up | 12 |
| 74 | ACAT2 | 39 | up | 12 |
| 75 | HOXA10 | 3206 | down | 12 |
| 76 | IL1B | 3553 | down | 12 |
| 77 | HK1 | 3098 | up | 13 |
| 78 | PSMD4 | 5710 | up | 13 |
| 79 | CLTC | 1213 | up | 13 |
| 80 | MAP4K4 | 9448 | up | 13 |
| 81 | PROS1 | 5627 | up | 14 |
| 82 | DLD | 1738 | up | 14 |
| 83 | SDHB | 6390 | up | 14 |
| 84 | ICAM3 | 3385 | down | 14 |
| 85 | GNAS | 2778 | up | 15 |
| 86 | COPS7A | 50813 | up | 15 |
| 87 | ADGRG1 | 9289 | down | 15 |
| 88 | HLA-DMA | 3108 | down | 15 |
| 89 | MPC2 | 25874 | up | 16 |
| 90 | HEBP1 | 50865 | up | 16 |
| 91 | BLVRA | 644 | up | 16 |
| 92 | ID2 | 3398 | up | 16 |
| 93 | SCAND1 | 51282 | up | 17 |
| 94 | ETFB | 2109 | up | 17 |
| 95 | MRPS16 | 51021 | up | 17 |
| 96 | PIN1 | 5300 | up | 18 |
| 97 | TRAK2 | 66008 | up | 18 |
| 98 | AMDHD2 | 51005 | up | 18 |
| 99 | PLEKHJ1 | 55111 | up | 19 |
| 100 | BZW2 | 28969 | up | 19 |
| 101 | PDLIM1 | 9124 | down | 19 |
| 102 | PCNA | 5111 | up | 20 |
| 103 | WDR61 | 80349 | up | 20 |
| 104 | RFC5 | 5985 | up | 20 |
| 105 | OXA1L | 5018 | up | 21 |
| 106 | MCM3 | 4172 | up | 21 |
| 107 | PSMB8 | 5696 | down | 21 |
| 108 | CEP57 | 9702 | up | 22 |
| 109 | PSMF1 | 9491 | up | 22 |
| 110 | POLR2K | 5440 | up | 22 |
| 111 | PSMD2 | 5708 | up | 23 |
| 112 | ATP6V1D | 51382 | up | 23 |
| 113 | PSMD9 | 5715 | up | 23 |
| 114 | AKAP8L | 26993 | up | 24 |
| 115 | GRN | 2896 | up | 24 |
| 116 | SPAG7 | 9552 | up | 24 |
| 117 | ENOSF1 | 55556 | up | 25 |
| 118 | PCK2 | 5106 | up | 25 |
| 119 | PCCB | 5096 | up | 25 |
| 120 | NOLC1 | 9221 | up | 26 |
| 121 | EBNA1BP2 | 10969 | up | 26 |
| 122 | EPB41L2 | 2037 | down | 26 |
| 123 | CD58 | 965 | up | 27 |
| 124 | RFC2 | 5982 | up | 27 |
| 125 | RPL39L | 116832 | down | 27 |
| 126 | ASAH1 | 427 | up | 28 |
| 127 | LAGE3 | 8270 | up | 28 |
| 128 | AKR7A2 | 8574 | up | 28 |
| 129 | RSU1 | 6251 | up | 29 |
| 130 | PYGL | 5836 | down | 29 |
| 131 | CYB561 | 1534 | down | 30 |
| 132 | HOMER2 | 9455 | down | 30 |

TABLE 1b
Genes showing a change in expression.

| | Gene | Gene_Entrez_ID | Network_Module |
|---|---|---|---|
| 0 | RAP1GAP | 5909 | 1 |
| 1 | E2F2 | 1870 | 1 |
| 2 | RSRP1 | 57035 | 1 |
| 3 | RHD | 6007 | 1 |
| 4 | RHCE | 6006 | 1 |
| 5 | ERMAP | 114625 | 1 |
| 6 | SLC2A1 | 6513 | 1 |
| 7 | CD58 | 965 | 1 |
| 8 | SELENBP1 | 8991 | 1 |
| 9 | PPOX | 3060 | 1 |
| 10 | NPL | 80896 | 1 |
| 11 | ADIPOR1 | 51094 | 1 |
| 12 | BTG2 | 7832 | 1 |
| 13 | KLHDC8A | 55220 | 1 |
| 14 | SDE2 | 163859 | 1 |
| 15 | GUK1 | 2987 | 1 |
| 16 | LBH | 221491 | 1 |
| 17 | LTBP1 | 4052 | 1 |
| 18 | ZC3H6 | 376940 | 1 |
| 19 | TRAK2 | 66008 | 1 |
| 20 | STRADB | 55437 | 1 |
| 21 | TMBIM1 | 64114 | 1 |
| 22 | DNAJB2 | 3300 | 1 |
| 23 | KAT2B | 8850 | 1 |
| 24 | ABHD5 | 51099 | 1 |
| 25 | CPOX | 1371 | 1 |
| 26 | RAB6B | 51560 | 1 |
| 27 | PAQR9 | 344838 | 1 |
| 28 | SIAH2 | 6478 | 1 |
| 29 | NCEH1 | 57552 | 1 |
| 30 | KLF3 | 51274 | 1 |
| 31 | FRYL | 285527 | 1 |
| 32 | MOB1B | 92597 | 1 |
| 33 | HERC6 | 55008 | 1 |
| 34 | TSPAN5 | 10098 | 1 |
| 35 | GYPE | 2996 | 1 |
| 36 | GYPB | 2994 | 1 |
| 37 | FHDC1 | 85462 | 1 |
| 38 | CLCN3 | 1182 | 1 |
| 39 | ANKH | 56172 | 1 |
| 40 | EPB41L4A | 64097 | 1 |
| 41 | IRF1 | 3659 | 1 |
| 42 | CYSTM1 | 84418 | 1 |
| 43 | FAXDC2 | 10826 | 1 |
| 44 | TRIM 10 | 10107 | 1 |
| 45 | TSPO2 | 222642 | 1 |
| 46 | CCND3 | 896 | 1 |
| 47 | GTPBP2 | 54676 | 1 |
| 48 | GCLC | 2729 | 1 |
| 49 | FOXO3 | 2309 | 1 |
| 50 | SERINC1 | 57515 | 1 |
| 51 | CITED2 | 10370 | 1 |
| 52 | JAZF1 | 221895 | 1 |
| 53 | MTURN | 222166 | 1 |
| 54 | CD36 | 948 | 1 |
| 55 | PNPLA8 | 50640 | 1 |
| 56 | BPGM | 669 | 1 |
| 57 | KDM7A | 80853 | 1 |
| 58 | MFHAS1 | 9258 | 1 |
| 59 | CTSB | 1508 | 1 |
| 60 | SLC25A37 | 51312 | 1 |
| 61 | BNIP3L | 665 | 1 |
| 62 | RNF19A | 25897 | 1 |
| 63 | GRINA | 2907 | 1 |
| 64 | HSF1 | 3297 | 1 |
| 65 | AQP3 | 360 | 1 |
| 66 | CTSL | 1514 | 1 |
| 67 | TMOD1 | 7111 | 1 |
| 68 | STOM | 2040 | 1 |
| 69 | RXRA | 6256 | 1 |
| 70 | OPTN | 10133 | 1 |
| 71 | FRMD4A | 55691 | 1 |
| 72 | STAM | 8027 | 1 |
| 73 | MXI1 | 4601 | 1 |
| 74 | UROS | 7390 | 1 |
| 75 | RIC8A | 60626 | 1 |
| 76 | ILK | 3611 | 1 |
| 77 | SOX6 | 55553 | 1 |
| 78 | CAT | 10249 | 1 |
| 79 | YPEL4 | 219539 | 1 |
| 80 | UCP2 | 7351 | 1 |
| 81 | PPME1 | 51400 | 1 |
| 82 | ENDOD1 | 23052 | 1 |
| 83 | PTMS | 5763 | 1 |
| 84 | CMAS | 55907 | 1 |
| 85 | NFE2 | 4778 | 1 |
| 86 | KIF5A | 3798 | 1 |
| 87 | RAB3IP | 117177 | 1 |
| 88 | NUDT4 | 11163 | 1 |
| 89 | HECTD4 | 283450 | 1 |
| 90 | SDSL | 113675 | 1 |
| 91 | RB1 | 5925 | 1 |
| 92 | PNP | 4860 | 1 |
| 93 | DCAF11 | 80344 | 1 |
| 94 | ATP6V1D | 51382 | 1 |
| 95 | DPF3 | 8110 | 1 |
| 96 | ZFYVE21 | 79038 | 1 |
| 97 | KIF26A | 26153 | 1 |
| 98 | KLF13 | 51621 | 1 |
| 99 | CCNDBP1 | 23582 | 1 |
| 100 | EPB42 | 2038 | 1 |
| 101 | REXO5 | 81691 | 1 |
| 102 | ITFG1 | 81533 | 1 |
| 103 | TERF2IP | 54386 | 1 |
| 104 | SLC7A5 | 8140 | 1 |
| 105 | EPN2 | 22905 | 1 |
| 106 | NATD1 | 256302 | 1 |
| 107 | PLEKHH3 | 79990 | 1 |
| 108 | SLC4A1 | 6521 | 1 |
| 109 | FAM117A | 81558 | 1 |
| 110 | WIPI1 | 55062 | 1 |
| 111 | SMIM5 | 643008 | 1 |
| 112 | LPIN2 | 9663 | 1 |
| 113 | RIOK3 | 8780 | 1 |
| 114 | UBXN6 | 80700 | 1 |
| 115 | 2-Mar | 51257 | 1 |
| 116 | JUNB | 3726 | 1 |
| 117 | AKAP8L | 26993 | 1 |
| 118 | UPK1A-AS1 | | 1 |
| 119 | PPP1R15A | 23645 | 1 |
| 120 | GPCPD1 | 56261 | 1 |
| 121 | FAM210B | 116151 | 1 |
| 122 | RBM38 | 55544 | 1 |
| 123 | TUBB1 | 81027 | 1 |
| 124 | ITSN1 | 6453 | 1 |
| 125 | GRAP2 | 9402 | 1 |
| 126 | WWC3 | 55841 | 1 |
| 127 | ALAS2 | 212 | 1 |
| 128 | FAM122C | 159091 | 1 |
| 129 | MOSPD1 | 56180 | 1 |
| 130 | CLSTN1 | 22883 | 8 |
| 131 | PGM1 | 5236 | 8 |
| 132 | CD2 | 914 | 8 |
| 133 | PHGDH | 26227 | 8 |
| 134 | MLLT11 | 10962 | 8 |
| 135 | IFI16 | 3428 | 8 |
| 136 | FCER1A | 2205 | 8 |
| 137 | RGS18 | 64407 | 8 |
| 138 | CD34 | 947 | 8 |
| 139 | NENF | 29937 | 8 |
| 140 | PLEK | 5341 | 8 |
| 141 | IL1B | 3553 | 8 |
| 142 | IGFBP7 | 3490 | 8 |
| 143 | INPP4B | 8821 | 8 |
| 144 | BASP1 | 10409 | 8 |
| 145 | FYB1 | 2533 | 8 |
| 146 | CD74 | 972 | 8 |
| 147 | SERPINB6 | 5269 | 8 |
| 148 | TUBB2B | 347733 | 8 |
| 149 | LTB | 4050 | 8 |
| 150 | LST1 | 7940 | 8 |
| 151 | AIF1 | 199 | 8 |
| 152 | MPIG6B | 80739 | 8 |
| 153 | HLA-DRA | 3122 | 8 |
| 154 | HLA-DRB1 | 3123 | 8 |
| 155 | HLA-DMA | 3108 | 8 |
| 156 | HLA-DPA1 | 3113 | 8 |
| 157 | HLA-DPB1 | 3115 | 8 |
| 158 | MAP7 | 9053 | 8 |
| 159 | CPVL | 54504 | 8 |
| 160 | SCRN1 | 9805 | 8 |
| 161 | GNAI1 | 2770 | 8 |
| 162 | CYP3A4 | 1576 | 8 |
| 163 | PRKAG2 | 51422 | 8 |
| 164 | CLU | 1191 | 8 |
| 165 | PVT1 | | 8 |
| 166 | ALDH1A1 | 216 | 8 |
| 167 | NFIL3 | 4783 | 8 |
| 168 | DNM1 | 1759 | 8 |
| 169 | FAM69B | 138311 | 8 |
| 170 | NPDC1 | 56654 | 8 |
| 171 | VIM | 7431 | 8 |
| 172 | ARID5B | 84159 | 8 |
| 173 | ZMIZ1 | 57178 | 8 |
| 174 | PRXL2A | 84293 | 8 |
| 175 | SPI1 | 6688 | 8 |
| 176 | DRAP1 | 10589 | 8 |
| 177 | PRCP | 5547 | 8 |
| 178 | PRSS23 | 11098 | 8 |
| 179 | TUBA1A | 7846 | 8 |
| 180 | FAM19A2 | 338811 | 8 |
| 181 | LYZ | 4069 | 8 |
| 182 | SLC22A17 | 51310 | 8 |
| 183 | CMTM5 | 116173 | 8 |
| 184 | NFATC4 | 4776 | 8 |
| 185 | HDC | 51696 | 8 |
| 186 | ANPEP | 290 | 8 |
| 187 | NPW | 283869 | 8 |
| 188 | ACSM3 | 6296 | 8 |
| 189 | CORO1A | 11151 | 8 |
| 190 | COTL1 | 23406 | 8 |
| 191 | CYBA | 1535 | 8 |
| 192 | ITM2BP1 | | 8 |
| 193 | ICAM3 | 3385 | 8 |
| 194 | HCST | 10870 | 8 |
| 195 | TYROBP | 7305 | 8 |
| 196 | PPP1R14A | 94274 | 8 |
| 197 | RN7SL555P | | 8 |
| 198 | APP | 351 | 8 |
| 199 | TIAM1 | 7074 | 8 |
| 200 | CBR3 | 874 | 8 |
| 201 | NCF4 | 4689 | 8 |
| 202 | CSF2RB | 1439 | 8 |
| 203 | BEX2 | 84707 | 8 |
| 204 | 6-Sep | 23157 | 8 |
| 205 | RAB33A | 9363 | 8 |
| 206 | SESN2 | 83667 | 17 |
| 207 | GADD45A | 1647 | 17 |
| 208 | DENND2D | 79961 | 17 |
| 209 | FAM91A3P | | 17 |
| 210 | S100A6 | 6277 | 17 |
| 211 | IER5 | 51278 | 17 |
| 212 | RGS16 | 6004 | 17 |
| 213 | PHLDA3 | 23612 | 17 |
| 214 | XPC | 7508 | 17 |
| 215 | LXN | 56925 | 17 |
| 216 | ZMAT3 | 64393 | 17 |
| 217 | CDKN1A | 1026 | 17 |
| 218 | SESN1 | 27244 | 17 |
| 219 | PLIN2 | 123 | 17 |
| 220 | RPS6 | 6194 | 17 |
| 221 | CDKN2A | 1029 | 17 |
| 222 | ANKRD18A | 253650 | 17 |
| 223 | LCN12 | 286256 | 17 |
| 224 | FAS | 355 | 17 |
| 225 | CTSD | 1509 | 17 |
| 226 | HBBP1 | | 17 |
| 227 | DDB2 | 1643 | 17 |
| 228 | CTTN | 2017 | 17 |
| 229 | SNORD15B | | 17 |
| 230 | MDM2 | 4193 | 17 |
| 231 | PXMP2 | 5827 | 17 |
| 232 | PLEK2 | 26499 | 17 |
| 233 | RPS27L | 51065 | 17 |
| 234 | LYRM1 | 57149 | 17 |
| 235 | RNF167 | 26001 | 17 |
| 236 | RNU4-34P | | 17 |
| 237 | MYL4 | 4635 | 17 |
| 238 | FDXR | 2232 | 17 |
| 239 | TNFSF9 | 8744 | 17 |
| 240 | CD70 | 970 | 17 |
| 241 | GDF15 | 9518 | 17 |
| 242 | ECH1 | 1891 | 17 |
| 243 | BBC3 | 27113 | 17 |
| 244 | BAX | 581 | 17 |
| 245 | FTL | 2512 | 17 |
| 246 | RPS5 | 6193 | 17 |
| 247 | ADA | 100 | 17 |
| 248 | GNAS | 2778 | 17 |
| 249 | APOBEC3H | 164668 | 17 |
| 250 | RHOC | 389 | 54 |
| 251 | CYP1B1 | 1545 | 54 |
| 252 | SUCNR1 | 56670 | 54 |
| 253 | TIPARP | 25976 | 54 |
| 254 | HSD17B11 | 51170 | 54 |
| 255 | HLA-A | 3105 | 54 |
| 256 | HLA-B | 3106 | 54 |
| 257 | PSMB9 | 5698 | 54 |
| 258 | ASAH1 | 427 | 54 |
| 259 | VPS28 | 51160 | 54 |
| 260 | HACD1 | 9200 | 54 |
| 261 | BGLT3 | | 54 |
| 262 | HBG1 | 3047 | 54 |
| 263 | HBG2 | 3048 | 54 |
| 264 | TRIM22 | 10346 | 54 |
| 265 | PRDX5 | 25824 | 54 |
| 266 | TSC22D1 | 8848 | 54 |
| 267 | RGS6 | 9628 | 54 |
| 268 | IFI27L2 | 83982 | 54 |
| 269 | B2M | 567 | 54 |
| 270 | ARID3A | 1820 | 54 |
| 271 | RABAC1 | 10567 | 54 |
| 272 | BEX1 | 55859 | 54 |

TABLE 2
Transcription factors showing a change in expression.

| | | | Gene_Di- |
|---|---|---|---|
| | Gene | Gene_Entrez_ID | rectionality |
| 0 | HMGA1 | 3159 | up |
| 1 | KLF1 | 10661 | up |
| 2 | KLF6 | 1316 | up |
| 3 | SREBF1 | 6720 | up |
| 4 | NFE2 | 4778 | up |
| 5 | ARID3A | 1820 | up |
| 6 | GFI1B | 8328 | up |
| 7 | KLF13 | 51621 | up |
| 8 | MLXIP | 22877 | up |
| 9 | E2F8 | 79733 | up |
| 10 | MYBL2 | 4605 | up |
| 11 | HSF1 | 3297 | up |
| 12 | GMEB1 | 10691 | up |
| 13 | NFX1 | 4799 | up |
| 14 | TGIF1 | 7050 | up |
| 15 | KLF3 | 51274 | up |
| 16 | SP1 | 199699 | up |
| 17 | CENPX | 201254 | up |
| 18 | HES6 | 55502 | up |
| 19 | LIN28B | 389421 | up |
| 20 | HOXA10 | 3206 | dn |
| 21 | XBP1 | 7494 | dn |
| 22 | SOX4 | 6659 | dn |
| 23 | ZNF385D | 79750 | dn |
| 24 | NFIC | 4782 | dn |
| 25 | BATF | 10538 | dn |
| 26 | HHEX | 3087 | dn |
| 27 | RARG | 5916 | dn |
| 28 | KDM5B | 10765 | dn |
| 29 | ZFX | 7543 | dn |
| 30 | SPI1 | 6688 | dn |
| 31 | TEAD4 | 7004 | dn |
| 32 | SATB1 | 6304 | dn |
| 33 | NFIX | 4784 | dn |
| 34 | PLAGL1 | 5325 | dn |
| 35 | MEF2C | 4208 | dn |
| 36 | ZBTB1 | 22890 | dn |
| 37 | HOXA9 | 3205 | dn |
| 38 | THAP5 | 168451 | dn |
| 39 | ZFP57 | 346171 | dn |

Table 1a, Table 1b, Table 2, and associated embodiments:
“Gene ID”: at the time of filing the present disclosure, the World Wide Web at ncbi.nlm.nih.gov/gene provides a description of and the nucleic acid sequence for each GeneID listed in Table 1a or Table 1b or Table 2, the contents of each of which is incorporated herein by reference in its entirety.
“Up” indicates a gene for which an increase in expression and/or activity in the progenitor cell is associated with the gene signature.
“Down” indicates a gene for which an decrease in expression and/or activity in the progenitor cell is associated with the gene signature.

A “network module” (sometimes also referred to as “module”) is a set of genes whose activity and/or expression are mutually predictive and, individually and collectively, are correlated with regard to a cell state change, which correlation may be positive or negative. That is, a module may contain genes that are positively associated with the cell state transition—such that an increase in expression and/or activity of the gene associated with the cell state transition; as well as genes that are negatively associated with the cell state transition such that a decrease in expression and/or activity of the gene associated with the cell state transition.

In certain embodiments, a network module includes genes in addition (or substituted for) to those exemplified in Table 1a, which should be viewed as illustrative and not limiting unless expressly provided, namely with genes with correlated expression. A correlation, e.g., by the method of Pearson or Spearman, is calculated between a query gene expression profile for the desired cell state transition and one or more of the exemplary genes recited in the module. Those genes with a correlation with one or more genes of the module of at significance level below p=0.05 (e.g., 0.04, 0.03, 0.02, 0.01, 0.005, 0.001, 0.0005, 0.0001, or less) can be added to, or substituted for, other genes in the module.

“Activation of a network module” refers to a perturbation that modulates expression and/or activity of 2 or more genes (e.g., 3, 4, 5, 6 . . . genes; or about 10, 20, 25, 30, 40, 50, 60, 70, 75, 80, 85, 90, 95, or 100%) within a module, which modulation may be an increase or decrease in expression and/or activity of the gene as consonant with the modules described in Table 1a. In certain embodiments, a perturbation activates multiple network modules for the desired cell state transition, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 modules. In certain embodiments, a perturbation activates at least one network modules for the desired cell state transition selected from network modules 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30 described in Table 1a. In certain embodiments, a perturbation activates at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 network modules for the desired cell state transition selected from network modules 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30 described in Table 1a. In certain embodiments, a perturbation activates each of the network modules 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30 described in Table 1a.

In some embodiments, one or more genes of network module 0 are modulated. In some embodiments, the perturbation activates network module 1, e.g., modulation of the expression and/or activity of one or more of (inclusive of all of) DDIT4, EPRS, MTHFD2, EIF4EBP1, AARS, ABCC5, PHGDH, NUCB2, and XBP1 genes. In some embodiments, the modulation is upmodulation or downmodulation as described in Gene Directionality column of Table 1a.

In some embodiments, one or more genes of network module 1 are modulated. In some embodiments, the perturbation activates network module 1, e.g., modulation of the expression and/or activity of one or more of (inclusive of all of) TUBB6, LSM6, EIF4G1, RNF167, CD320, CCNB1, CDC20, and PLK1 genes. In some embodiments, the modulation is upmodulation or downmodulation as described in Gene Directionality column of Table 1a.

In some embodiments, one or more genes of network module 2 are modulated. In some embodiments, the perturbation activates network module 2, e.g., modulation of the expression and/or activity of one or more of (inclusive of all of) CTNNAL1, GADD45A, PTK2, CFLAR, IGF2BP2, CDK6, and ITGB1BP1 genes. In some embodiments, the modulation is upmodulation or downmodulation as described in Gene Directionality column of Table 1a.

In some embodiments, one or more genes of network module 3 are modulated. In some embodiments, the perturbation activates network module 3, e.g., modulation of the expression and/or activity of one or more of (inclusive of all of) CDK1, CDC45, CDCA4, MELK, HAT1, and CCNE2 genes. In some embodiments, the modulation is upmodulation or downmodulation as described in Gene Directionality column of Table 1a.

In some embodiments, one or more genes of network module 4 are modulated. In some embodiments, the perturbation activates network module 4, e.g., modulation of the expression and/or activity of one or more of (inclusive of all of) PAK1, TSPAN6, TIMM17B, KDM5A, UBE3B, and PTPN6 genes. In some embodiments, the modulation is upmodulation or downmodulation as described in Gene Directionality column of Table 1a.

In some embodiments, one or more genes of network module 5 are modulated. In some embodiments, the perturbation activates network module 5, e.g., modulation of the expression and/or activity of one or more of (inclusive of all of) RPS5, PAICS, RPIA, KDELR2, PNP, and CBR1 genes. In some embodiments, the modulation is upmodulation or downmodulation as described in Gene Directionality column of Table 1a.

In some embodiments, one or more genes of network module 6 are modulated. In some embodiments, the perturbation activates network module 6, e.g., modulation of the expression and/or activity of one or more of (inclusive of all of) CAST, H2AFV, ATP11B, CTNND1, ORC1, and HLA-DRA genes. In some embodiments, the modulation is upmodulation or downmodulation as described in Gene Directionality column of Table 1a.

In some embodiments, one or more genes of network module 7 are modulated. In some embodiments, the perturbation activates network module 7, e.g., modulation of the expression and/or activity of one or more of (inclusive of all of) FDFT1, CDKN1B, INSIG1, IGF1R, and MAP7 genes. In some embodiments, the modulation is upmodulation or downmodulation as described in Gene Directionality column of Table 1a.

In some embodiments, one or more genes of network module 8 are modulated. In some embodiments, the perturbation activates network module 8, e.g., modulation of the expression and/or activity of one or more of (inclusive of all of) TRAP1, TSTA3, SUZ12, CDK4, and SOX4 genes. In some embodiments, the modulation is upmodulation or downmodulation as described in Gene Directionality column of Table 1a.

In some embodiments, one or more genes of network module 9 are modulated. In some embodiments, the perturbation activates network module 9, e.g., modulation of the expression and/or activity of one or more of (inclusive of all of) HMGCS1, LAP3, TBPL1, FAH, and CCP110 genes. In some embodiments, the modulation is upmodulation or downmodulation as described in Gene Directionality column of Table 1a.

In some embodiments, one or more genes of network module 10 are modulated. In some embodiments, the perturbation activates network module 10, e.g., modulation of the expression and/or activity of one or more of (inclusive of all of) APOE, IGF2R, DYRK3, CASP3, and DNAJB6 genes. In some embodiments, the modulation is upmodulation or downmodulation as described in Gene Directionality column of Table 1a.

In some embodiments, one or more genes of network module 11 are modulated. In some embodiments, the perturbation activates network module 11, e.g., modulation of the expression and/or activity of one or more of (inclusive of all of) MYBL2, APP, DNMT1, SMC3, and HTATSF1 genes. In some embodiments, the modulation is upmodulation or downmodulation as described in Gene Directionality column of Table 1a.

In some embodiments, one or more genes of network module 12 are modulated. In some embodiments, the perturbation activates network module 12, e.g., modulation of the expression and/or activity of one or more of (inclusive of all of) CAT, ACAT2, HOXA10, and MB genes. In some embodiments, the modulation is upmodulation or downmodulation as described in Gene Directionality column of Table 1a.

In some embodiments, one or more genes of network module 13 are modulated. In some embodiments, the perturbation activates network module 13, e.g., modulation of the expression and/or activity of one or more of (inclusive of all of) HK1, PSMD4, CLTC, and MAP4K4 genes. In some embodiments, the modulation is upmodulation or downmodulation as described in Gene Directionality column of Table 1a.

In some embodiments, one or more genes of network module 14 are modulated. In some embodiments, the perturbation activates network module 14, e.g., modulation of the expression and/or activity of one or more of (inclusive of all of) PROS1, DLD, SDHB, and ICAM3 genes. In some embodiments, the modulation is upmodulation or downmodulation as described in Gene Directionality column of Table 1a.

In some embodiments, one or more genes of network module 15 are modulated. In some embodiments, the perturbation activates network module 15, e.g., modulation of the expression and/or activity of one or more of (inclusive of all of) GNAS, COPS7A, ADGRG1, and HLA-DMA genes. In some embodiments, the modulation is upmodulation or downmodulation as described in Gene Directionality column of Table 1a.

In some embodiments, one or more genes of network module 16 are modulated. In some embodiments, the perturbation activates network module 16, e.g., modulation of the expression and/or activity of one or more of (inclusive of all of) MPC2, HEBP1, BLVRA, and ID2 genes. In some embodiments, the modulation is upmodulation or downmodulation as described in Gene Directionality column of Table 1a.

In some embodiments, one or more genes of network module 17 are modulated. In some embodiments, the perturbation activates network module 17, e.g., modulation of the expression and/or activity of one or more of (inclusive of all of) SCAND1, ETFB, and MRPS16 genes. In some embodiments, the modulation is upmodulation or downmodulation as described in Gene Directionality column of Table 1a.

In some embodiments, one or more genes of network module 18 are modulated. In some embodiments, the perturbation activates network module 18, e.g., modulation of the expression and/or activity of one or more of (inclusive of all of) PIN1, TRAK2, and AMDHD2 genes. In some embodiments, the modulation is upmodulation or downmodulation as described in Gene Directionality column of Table 1a.

In some embodiments, one or more genes of network module 19 are modulated. In some embodiments, the perturbation activates network module 19, e.g., modulation of the expression and/or activity of one or more of (inclusive of all of) PLEKHJ1, BZW2, and PDLIM1 genes. In some embodiments, the modulation is upmodulation or downmodulation as described in Gene Directionality column of Table 1a.

In some embodiments, one or more genes of network module 20 are modulated. In some embodiments, the perturbation activates network module 20, e.g., modulation of the expression and/or activity of one or more of (inclusive of all of) PCNA, WDR61, and RFC5 genes. In some embodiments, the modulation is upmodulation or downmodulation as described in Gene Directionality column of Table 1a.

In some embodiments, one or more genes of network module 21 are modulated. In some embodiments, the perturbation activates network module 21, e.g., modulation of the expression and/or activity of one or more of (inclusive of all of) OXA1L, MCM3, and PSMB8 genes. In some embodiments, the modulation is upmodulation or downmodulation as described in Gene Directionality column of Table 1a.

In some embodiments, one or more genes of network module 22 are modulated. In some embodiments, the perturbation activates network module 22, e.g., modulation of the expression and/or activity of one or more of (inclusive of all of) CEP57, PSMF1, and POLR2K genes. In some embodiments, the modulation is upmodulation or downmodulation as described in Gene Directionality column of Table 1a.

In some embodiments, one or more genes of network module 23 are modulated. In some embodiments, the perturbation activates network module 23, e.g., modulation of the expression and/or activity of one or more of (inclusive of all of) PSMD2, ATP6V1D, and PSMD9 genes. In some embodiments, the modulation is upmodulation or downmodulation as described in Gene Directionality column of Table 1a.

In some embodiments, one or more genes of network module 24 are modulated. In some embodiments, the perturbation activates network module 24, e.g., modulation of the expression and/or activity of one or more of (inclusive of all of) AKAP8L, GRN, and SPAG7 genes. In some embodiments, the modulation is upmodulation or downmodulation as described in Gene Directionality column of Table 1a.

In some embodiments, one or more genes of network module 25 are modulated. In some embodiments, the perturbation activates network module 25, e.g., modulation of the expression and/or activity of one or more of (inclusive of all of) ENOSF1, PCK2, and PCCB genes. In some embodiments, the modulation is upmodulation or downmodulation as described in Gene Directionality column of Table 1a.

In some embodiments, one or more genes of network module 26 are modulated. In some embodiments, the perturbation activates network module 26, e.g., modulation of the expression and/or activity of one or more of (inclusive of all of) NOLC1, EBNA1BP2, and EPB41L2 genes. In some embodiments, the modulation is upmodulation or downmodulation as described in Gene Directionality column of Table 1a.

In some embodiments, one or more genes of network module 27 are modulated. In some embodiments, the perturbation activates network module 27, e.g., modulation of the expression and/or activity of one or more of (inclusive of all of) CD58, RFC2, and RPL39L genes. In some embodiments, the modulation is upmodulation or downmodulation as described in Gene Directionality column of Table 1a.

In some embodiments, one or more genes of network module 28 are modulated. In some embodiments, the perturbation activates network module 28, e.g., modulation of the expression and/or activity of one or more of (inclusive of all of) ASAH1, LAGE3, and AKR7A2 genes. In some embodiments, the modulation is upmodulation or downmodulation as described in Gene Directionality column of Table 1a.

In some embodiments, one or more genes of network module 29 are modulated. In some embodiments, the perturbation activates network module 29, e.g., modulation of the expression and/or activity of one or more of (inclusive of all of) RSU1 and PYGL genes. In some embodiments, the modulation is upmodulation or downmodulation as described in Gene Directionality column of Table 1a.

In some embodiments, one or more genes of network module 30 are modulated. In some embodiments, the perturbation activates network module 30, e.g., modulation of the expression and/or activity of one or more of (inclusive of all of) CYB561 and HOMER2 genes. In some embodiments, the modulation is upmodulation or downmodulation as described in Gene Directionality column of Table 1a.

In some embodiments, the present methods alter a gene signature in the sample of cells, comprising an activation of a network module designated in the network module column of Table 1a.

In some embodiments, the activation of the network module designated in the network module column of Table 1a comprises modulating expression and/or activity of 2 or more genes within a network module.

In some embodiments, the activation of the network module designated in the network module column of Table 1a comprises modulating expression and/or activity of all of the genes within a network module.

In some embodiments, the activation of the network module designated in the network module column of Table 1a comprises modulating expression and/or activity of 2 or more genes within 2 or more network modules. In some embodiments, the activation of the network module designated in the network module column of Table 1a comprises modulating expression and/or activity of 2 or more genes (e.g. 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or 41 or more, or 42 or more, or 43 or more, or 44 or more, or 45 or more, or 46 or more, or 47 or more, or 48 or more, or 49 or more, 50 or more, 51 or more, or 52 or more, or 53 or more, or 54 or more, or 55 or more, or 56 or more, or 57 or more, or 58 or more, or 59 or more, 60 or more, or 61 or more, or 62 or more, or 63 or more, or 64 or more, or 65 or more, or 66 or more, or 67 or more, or 68 or more, or 69 or more, 70 or more, or 71 or more, or 72 or more, or 73 or more, or 74 or more, or 75 or more, or 76 or more, or 77 or more, or 78 or more, or 79 or more, 80 or more, or 81 or more, or 82 or more, or 83 or more, or 84 or more, or 85 or more, or 86 or more, or 87 or more, or 88 or more, or 89 or more, 90 or more, or 91 or more, or 92 or more, or 93 or more, or 94 or more, or 95 or more, or 96 or more, or 97 or more, or 98 or more, or 99 or more, or 100 or more, or 101 or more, or 102 or more, or 103 or more, or 104 or more, or 105 or more, or 106 or more, or 107 or more, or 108 or more, or 109 or more, or 110 or more, or 111 or more, or 112 or more, or 113 or more, or 114 or more, or 115 or more, or 116 or more, or 117 or more, or 118 or more, or 119 or more, or 120 or more, or 121 or more, or 122 or more, or 123 or more, or 124 or more, or 125 or more, or 126 or more, or 127 or more, or 128 or more, or 129 or more, or 130 or more, or 131 or more, or 132 or more genes) within 2 or more network modules (e.g. 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more network modules selected from the network modules 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30 described in Table 1a).

In certain embodiments, a network module includes genes in addition (or substituted for) to those exemplified in Table 1b, which should be viewed as illustrative and not limiting unless expressly provided, namely with genes with correlated expression. A correlation, e.g., by the method of Pearson or Spearman, is calculated between a query gene expression profile for the desired cell state transition and one or more of the exemplary genes recited in the module. Those genes with a correlation with one or more genes of the module of at significance level below p=0.05 (e.g., 0.04, 0.03, 0.02, 0.01, 0.005, 0.001, 0.0005, 0.0001, or less) can be added to, or substituted for, other genes in the module.

“Activation of a network module” refers to a perturbation that modulates expression and/or activity of 2 or more genes (e.g., 3, 4, 5, 6 . . . genes; or about 10, 20, 25, 30, 40, 50, 60, 70, 75, 80, 85, 90, 95, or 100%) within a module, which modulation may be an increase or decrease in expression and/or activity of the gene as consonant with the modules described in Table 1b. In certain embodiments, a perturbation activates multiple network modules for the desired cell state transition, such as 2, 3, or 4 modules. In certain embodiments, a perturbation activates at least one network modules for the desired cell state transition selected from network modules 1, 8, 17, and 54 described in Table 1b. In certain embodiments, a perturbation activates at least 2, 3, or 4 network modules for the desired cell state transition selected from network modules 1, 8, 17, and 54 described in Table 1b. In certain embodiments, a perturbation activates each of the network modules 1, 8, 17, and 54 described in Table 1b.

In some embodiments, one or more genes of network module 1 are modulated. In some embodiments, the perturbation activates network module 1, e.g., modulation of the expression and/or activity of one or more of (inclusive of all of) RAP1GAP, E2F2, RSRP1, RHD, RHCE, ERMAP, SLC2A1, CD58, SELENBP1, PPOX, NPL, ADIPOR1, BTG2, KLHDC8A, SDE2, GUK1, LBH, LTBP1, ZC3H6, TRAK2, STRADB, TMBIM1, DNAJB2, KAT2B, ABHD5, CPOX, RAB6B, PAQR9, SIAH2, NCEH1, KLF3, FRYL, MOB1B, HERC6, TSPAN5, GYPE, GYPB, FHDC1, CLCN3, ANKH, EPB41L4A, IRF1, CYSTM1, FAXDC2, TRIM10, TSPO2, CCND3, GTPBP2, GCLC, FOXO3, SERINC1, CITED2, JAZF1, MTURN, CD36, PNPLA8, BPGM, KDM7A, MFHAS1, CTSB, SLC25A37, BNIP3L, RNF19A, GRINA, HSF1, AQP3, CTSL, TMOD1, STOM, RXRA, OPTN, FRMD4A, STAM, MXI1, UROS, RIC8A, ILK, SOX6, CAT, YPEL4, UCP2, PPME1, ENDOD1, PTMS, CMAS, NFE2, KIF5A, RAB3IP, NUDT4, HECTD4, SDSL, RB1, PNP, DCAF11, ATP6V1D, DPF3, ZFYVE21, KIF26A, KLF13, CCNDBP1, EPB42, REXO5, ITFG1, TERF2IP, SLC7A5, EPN2,NATD1, PLEKHH3, SLC4A1, FAM117A, WIPI1, SMIM5, LPIN2, RIOK3, UBXN6, 2-Mar, JUNB, AKAP8L, UPK1A-AS1, PPP1R15A, GPCPD1, FAM210B, RBM38, TUBB1, ITSN1, GRAP2, WWC3, ALAS2, FAM122C, and MOSPD1 genes. In some embodiments, the modulation is upmodulation or downmodulation.

In some embodiments, one or more genes of network module 8 are modulated. In some embodiments, the perturbation activates network module 8, e.g., modulation of the expression and/or activity of one or more of (inclusive of all of) SOX4, CLSTN1, PGM1, CD2, PHGDH, MLLT11, IFI16, FCER1A, RGS18, CD34, NENF, PLEK, IL1B, IGFBP7, INPP4B, BASP1, FYB1, CD74, SERPINB6, TUBB2B, LTB, LST1, AIF1, MPIG6B, HLA-DRA, HLA-DRB1, HLA-DMA, HLA-DPA1, HLA-DPB1, MAP7, CPVL, SCRN1, GNAI1, CYP3A4, PRKAG2, CLU, PVT1, ALDH1A1, NFIL3, DNM1, FAM69B, NPDC1, VIM, ARID5B, and ZMIZ1 genes. In some embodiments, the modulation is upmodulation or downmodulation.

In some embodiments, one or more genes of network module 17 are modulated. In some embodiments, the perturbation activates network module 17, e.g., modulation of the expression and/or activity of one or more of (inclusive of all of) SESN2, GADD45A, DENND2D, FAM91A3P, S100A6, IER5, RGS16, PHLDA3, XPC, LXN, ZMAT3, CDKN1A, SESN1, PLIN2, RPS6, CDKN2A, ANKRD18A, LCN12, FAS, CTSD, HBBP1, DDB2, CTTN, SNORD15B, MDM2, PXMP2, PLEK2, RPS27L, LYRM1, RNF167, RNU4-34P, MYL4, FDXR, TNFSF9, CD70, GDF15, ECH1, BBC3, BAX, FTL, RPS5, ADA, GNAS, and APOBEC3H genes. In some embodiments, the modulation is upmodulation or downmodulation.

In some embodiments, one or more genes of network module 54 are modulated. In some embodiments, the perturbation activates network module 54, e.g., modulation of the expression and/or activity of one or more of (inclusive of all of) RHOC, CYP1B1, SUCNR1, TIPARP, HSD17B11, HLA-A, HLA-B, PSMB9, ASAH1, VPS28, HACD1, BGLT3, HBG1, HBG2, TRIM22, PRDX5, TSC22D1, RGS6, IFI27L2, B2M, ARID3A, RABAC1, and BEX1 genes. In some embodiments, the modulation is upmodulation or downmodulation.

In some embodiments, the present methods alter a gene signature in the sample of cells, comprising an activation of a network module designated in the network module column of Table 1b.

In some embodiments, the activation of the network module designated in the network module column of Table 1b comprises modulating expression and/or activity of 2 or more genes within a network module.

In some embodiments, the activation of the network module designated in the network module column of Table 1b comprises modulating expression and/or activity of all of the genes within a network module.

In some embodiments, the activation of the network module designated in the network module column of Table 1b comprises modulating expression and/or activity of 2 or more genes within 2 or more network modules. In some embodiments, the activation of the network module designated in the network module column of Table 1b comprises modulating expression and/or activity of 2 or more genes (e.g. 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or 41 or more, or 42 or more, or 43 or more, or 44 or more, or 45 or more, or 46 or more, or 47 or more, or 48 or more, or 49 or more, 50 or more, 51 or more, or 52 or more, or 53 or more, or 54 or more, or 55 or more, or 56 or more, or 57 or more, or 58 or more, or 59 or more, 60 or more, or 61 or more, or 62 or more, or 63 or more, or 64 or more, or 65 or more, or 66 or more, or 67 or more, or 68 or more, or 69 or more, 70 or more, or 71 or more, or 72 or more, or 73 or more, or 74 or more, or 75 or more, or 76 or more, or 77 or more, or 78 or more, or 79 or more, 80 or more, or 81 or more, or 82 or more, or 83 or more, or 84 or more, or 85 or more, or 86 or more, or 87 or more, or 88 or more, or 89 or more, 90 or more, or 91 or more, or 92 or more, or 93 or more, or 94 or more, or 95 or more, or 96 or more, or 97 or more, or 98 or more, or 99 or more, or 100 or more, or 101 or more, or 102 or more, or 103 or more, or 104 or more, or 105 or more, or 106 or more, or 107 or more, or 108 or more, or 109 or more, or 110 or more, or 111 or more, or 112 or more, or 113 or more, or 114 or more, or 115 or more, or 116 or more, or 117 or more, or 118 or more, or 119 or more, or 120 or more, or 121 or more, or 122 or more, or 123 or more, or 124 or more, or 125 or more, or 126 or more, or 127 or more, or 128 or more, or 129 or more, or 130 or more, or 131 or more, or 132 or more, 133 or more, or 134 or more, or 135 or more, or 136 or more, or 137 or more, or 138 or more, or 139 or more, or 140 or more, or 141 or more, or 142 or more, or 143 or more, or 144 or more, or 145 or more, or 146 or more, or 147 or more, or 148 or more, or 149 or more, 150 or more, 151 or more, or 152 or more, or 153 or more, or 154 or more, or 155 or more, or 156 or more, or 157 or more, or 158 or more, or 159 or more, 160 or more, or 161 or more, or 162 or more, or 163 or more, or 164 or more, or 165 or more, or 166 or more, or 167 or more, or 168 or more, or 169 or more, 170 or more, or 171 or more, or 172 or more, or 173 or more, or 174 or more, or 175 or more, or 176 or more, or 177 or more, or 178 or more, or 179 or more, 180 or more, or 181 or more, or 182 or more, or 183 or more, or 184 or more, or 185 or more, or 186 or more, or 187 or more, or 188 or more, or 189 or more, 190 or more, or 191 or more, or 192 or more, or 193 or more, or 194 or more, or 195 or more, or 196 or more, or 197 or more, or 198 or more, or 199 or more, or 200 or more, or 201 or more, or 202 or more, or 203 or more, or 204 or more, or 205 or more, or 206 or more, or 207 or more, or 208 or more, or 209 or more, or 210 or more, or 211 or more, or 212 or more, or 213 or more, or 214 or more, or 215 or more, or 216 or more, or 217 or more, or 218 or more, or 219 or more, or 220 or more, or 221 or more, or 222 or more, or 223 or more, or 224 or more, or 225 or more, or 226 or more, or 227 or more, or 228 or more, or 229 or more, or 230 or more, or 231 or more, or 232 or more, or 233 or more, or 234 or more, or 235 or more, or 236 or more, or 237 or more, or 238 or more, or 239 or more, or 240 or more, or 241 or more, or 242 or more, or 243 or more, or 244 or more, or 245 or more, or 246 or more, or 247 or more, or 248 or more, or 249 or more, 250 or more, 251 or more, or 252 or more, or 253 or more, or 254 or more, or 255 or more, or 256 or more, or 257 or more, or 258 or more, or 259 or more, 260 or more, or 261 or more, or 262 or more, or 263 or more, or 264 or more, or 265 or more, or 266 or more, or 267 or more, or 268 or more, or 269 or more, 270 or more, or 271 or more, or 272 or more genes) within 2 or more network modules (e.g. 2 or more, or 3 or more network modules selected from the network modules 1, 8, 17, and 54 described in Table 1b). At the time of filing the present disclosure, the World Wide Web at ncbi.nlm.nih.gov/gene provides a description of and the nucleic acid sequence for each Gene designated as an “up” gene in the gene directionality column of Table 1a and/or Table 2; the contents of each of which is incorporated herein by reference in its entirety. At the time of filing the present disclosure, the World Wide Web at ncbi.nlm.nih.gov/gene provides a description of and the nucleic acid sequence for each Gene listed in the genes designated as a “down” gene in the gene directionality column of Table 1a and/or Table 2; the contents of each of which is incorporated herein by reference in its entirety.

### Perturbagens

A perturbagen useful in the present disclosure can be a small molecule, a biologic, a protein, a nucleic acid, such as a cDNA over-expressing a wild-type gene or an mRNA encoding a wild-type gene, or any combination of any of the foregoing. Illustrative perturbagens useful in the present disclosure and capable of promoting erythrocyte lineage differentiation are listed in Table 3.

TABLE 3
Perturbagens

| Perturbagen | Molecular | Molecular Weight | Effective in vitro | |
|---|---|---|---|---|
| No. | Formula | (g/mol) | concentration | |
| 1 | C₃₁H₃₁ClFN₇O₂ | 588.1 | 10 | μM |
| 2 | C₁₈H₁₄Cl₄N₂O | 416.1 | 0.04 | um |
| 3 | C₂₈H₃₁N₃O₆ | 505.6 | 10.0 | um |
| 4 | C₂₃H₂₇N₇O₃S₂ | 513.6 | 3.33 | um |
| 5 | C₂₂H₂₉FO₄ | 376.5 | 10 | μM |
| 6 | C₂₃H₂₈N₄O₂ | 392.5 | 10 | μM |
| 7 | C₂₅H₃₂ClFO₅ | 467 | 3.33 | um |
| 8 | C₃₀H₃₃N₇O | 507.6 | 10 | μM |
| 9 | C₄₁H₆₄O₁₄ | 780.9 | 10 | μM |
| 10 | C₁₁H₁₅NO₅ | 241.24 | 10 | μM |
| 11 | C₉H₅Cl₂NO | 214.04 | 10 | μM |
| 12 | C₁₆H₁₃N₃O₃ | 295.29 | 1 | μM |
| 13 | C₁₉H₁₉NOS | 309.4 | 10.0 | um |
| 14 | C₁₆H₁₉ClN₂ | 274.79 | 10 | μM |
| 15 | C₂₁H₂₅N₅O₄S | 443.5 | 0.12 | um |
| 16 | C₂₅H₃₁N₅O₄ | 465.5 | 10 | μM |
| 17 | C₁₃H₁₅N₃O₂S | 277.34 | 10 | μM |
| 18 | C₁₄H₁₅ClN₆O | 318.76 | 10 | μM |
| 19 | C₈H₇NO₂S | 181.21 | 10 | μM |
| 20 | C₃₂H₄₈N₄O₈ | 616.7 | 5 | μM |
| 21 | C₂₁H₁₉FN₄O | 362.4 | 3.5 | um |
| 22 | C₈H₇ClN₂O₂S | 230.67 | 10 | μM |
| 23 | C₂₃H₁₃Cl₂N₃O₂ | 434.3 | 0.04 | um |
| 24 | C₂₄H₂₉N₃O₃ | 407.5 | 10 | μM |
| 25 | C₂₀H₁₄ClN₃O₃S | 411.9 | 0.37 | um |
| 26 | C₁₉H₂₁NO₅S | 375.4 | 10 | μM |
| 27 | C₁₁H₁₁F₃N₂O₃ | 276.21 | 10.0 | um |
| 28 | C₁₄H₁₀F₃NO₂ | 281.23 | 10 | μM |
| 29 | C₂₇H₃₂N₄O₇S | 556.6 | 10 | μM |
| 30 | C₂₈H₂₅FN₆O₃ | 512.5 | 0.12 | um |
| 31 | C₃₆H₅₆O₈ | 616.8 | 10 | μM |
| 32 | C₂₀H₂₄N₂O₃ | 340.4 | 10 | μM |
| 33 | C₂₁H₂₈BN₃O₅ | 413.3 | 0.12 | um |
| 34 | C₂₇H₄₁NO₆S | 507.7 | 1.11 | um |
| 35 | C₄₇H₅₁NO₁₄ | 853.9 | 500 | nM |
| 36 | C₁₅H₂₀O₃ | 248.32 | 10 | μM |
| 37 | C₂₇H₂₉NO₁₁ | 543.5 | 3 | μM |
| 38 | C₂₀H₂₈O₃ | 316.4 | 10 | μM |
| 39 | C₂₉H₄₀N₂O₄ | 480.6 | 500 | nM |
| 40 | C₃₃H₃₄N₄O₄S | 582.7 | 0.1 | um |
| 41 | C₁₅H₂₃NO₄ | 281.35 | 0.1 | um |
| 42 | C₄₂H₆₈N₆O₆S | 785.1 | 10.0 | um |

In various embodiments herein, a perturbagen of Table 3 encompasses the perturbagens named in Table 3. Thus, the named perturbagens of Table 3 represent examples of perturbagens of the present disclosure.

In Table 3, the effective in vitro concentration is the concentration of a perturbagen that is capable of increasing gene expression in a progenitor cell, as assayed, at least, by single cell gene expression profiling (GEP). Although the concentrations were determined in an in vitro assay, the concentrations may be relevant to a determination of in vivo dosages and such dosages may be used in clinic or in clinical testing.

In some embodiments, a perturbagen used in the present disclosure is a variant of a perturbagen of Table 3. A variant may be a derivative, analog, enantiomer or a mixture of enantiomers thereof or a pharmaceutically acceptable salt, solvate, hydrate, co-crystal, clathrate, or polymorph of the perturbagen of Table 3. A variant of a perturbagen of Table 3 retains the biological activity of the perturbagen of Table 3.

### Methods and Perturbagens for Directing a Change in Cell State

Particular cellular changes in cell state can be matched to differential gene expression, caused by exposure of a cell to a perturbagen. In some embodiments, a change in cell state may be from one progenitor cell type to another progenitor cell type. For example, a megakaryocyte-erythroid progenitor cell (MEP) may give rise to an erythrocyte. In some embodiments, a change in cell state may be from an upstream progenitor cell (e.g. proerythroblasts) to a downstream progenitor cell (e.g., late erythroblasts). Lastly, in some embodiments, a change in cell state may be from the final non-differentiated cell into a differentiated cell.

An aspect of the present disclosure is related to a method for directing a change in cell state of a progenitor cell. This method includes a step of contacting (e.g. in vitro or in vivo) a population of cells comprising a progenitor cell with at least one perturbagen selected from Table 3, or a variant of perturbagens described in Table 3. In this aspect, the at least one perturbagen is capable of altering a gene signature in the progenitor cell.

In one embodiment, the progenitor cell is a non-lineage committed CD34⁺ cell. In one embodiment, the progenitor cell is a non-lineage committed CD34⁺ cell. In one embodiment, the progenitor cell is a non-lineage committed CD34⁺ cells selected from a hematopoietic stem cell (an HSC; e.g., a CD34+ HSC), a burst-forming unit-erythroid (BFU-E) cell, a colony forming unit-erythroid (CFU-E) cell, a proerythroblast, a basophilic erythroblast (also known as an early erythroblast), and a polychromatic erythroblast (also known as an intermediate erythroblast), a orthochromatic erythroblast (also known as a late erythroblasts).

Another aspect of the present disclosure is related to a method for directing a change in cell state of a progenitor cell. This method includes a step of contacting a population of cells comprising a progenitor cell with at least one perturbagen capable of altering a gene signature in the progenitor cell. In this aspect, altering the gene signature comprises an increase in expression and/or activity in the progenitor cell of one or more genes selected from the genes designated as an “up” gene in the gene directionality column of Table 1a and/or Table 2 and/or a decrease in expression and/or activity in the progenitor cell of one or more genes selected from the genes designated as a “down” gene in the gene directionality column of Table 1a and/or Table 2 and/or an increase in expression and/or activity in the progenitor cell of one or more genes selected from Table 1b and/or a decrease in expression and/or activity in the progenitor cell of one or more genes selected from Table 1b. In one embodiment, the progenitor cell is a non-lineage committed CD34⁺ cell. In some embodiments, altering the gene signature further comprises increasing the expression of one or more transcription factors of the gene.

Yet another aspect of the present disclosure is related to a method for directing a change in cell state of a progenitor cell. This method includes a step of contacting a population of cells comprising a progenitor cell with at least one perturbagen selected from Table 3, or a variant of perturbagens described in Table 3, and capable of altering a gene signature in the progenitor cell. In this aspect, altering the gene signature comprises an increase in expression and/or activity in the progenitor cell of one or more genes selected from the genes designated as an “up” gene in the gene directionality column of Table 1a and/or Table 2 and/or a decrease in expression and/or activity in the progenitor cell of one or more genes selected from the genes designated as a “down” gene in the gene directionality column of Table 1a and/or Table 2 and/or an increase in expression and/or activity in the progenitor cell of one or more genes selected from Table 1b and/or a decrease in expression and/or activity in the progenitor cell of one or more genes selected from Table 1b. In one embodiment, the progenitor cell is a non-lineage committed CD34⁺ cell. In one embodiment, the progenitor cell is a non-lineage committed CD34⁺ cell. In one embodiment, the progenitor cell is a non-lineage committed CD34⁺ cells selected from a hematopoietic stem cell (an HSC; e.g., a CD34+ HSC), a burst-forming unit-erythroid (BFU-E) cell, a colony forming unit-erythroid (CFU-E) cell, a proerythroblast, a basophilic erythroblast (also known as an early erythroblast), a polychromatic erythroblast (also known as an intermediate erythroblast), and a orthochromatic erythroblast (also known as a late erythroblasts). In some embodiments, altering the gene signature further comprises increasing the expression of a transcription factor of the gene.

Yet another aspect of the present disclosure is related to a method for directing a change in cell state of a progenitor cell. This method includes a step of contacting a population of cells comprising a progenitor cell with at least one perturbagen selected from Table 3, or a variant of perturbagens described in Table 3, and capable of altering a gene signature in the progenitor cell. In this aspect, altering the gene signature comprises an increase in expression and/or activity in the progenitor cell of one or more genes selected from the genes designated as an “up” gene in the gene directionality column of Table 1a and/or Table 2 and/or a decrease in expression and/or activity in the progenitor cell of one or more genes selected from the genes designated as a “down” gene in the gene directionality column of Table 1a and/or Table 2 and/or an increase in expression and/or activity in the progenitor cell of one or more genes selected from Table 1b and/or a decrease in expression and/or activity in the progenitor cell of one or more genes selected from Table 1b. In one embodiment, the progenitor cell is a non-lineage committed CD34⁺ cell. In one embodiment, the progenitor cell is a non-lineage committed CD34⁺ cell. In one embodiment, the progenitor cell is a non-lineage committed CD34⁺ cells selected from a hematopoietic stem cell (an HSC; e.g., a CD34+ HSC), a burst-forming unit-erythroid (BFU-E) cell, a colony forming unit-erythroid (CFU-E) cell, a proerythroblast, a basophilic erythroblast (also known as an early erythroblast), a polychromatic erythroblast (also known as an intermediate erythroblast), and a orthochromatic erythroblast (also known as a late erythroblasts). In some embodiments, altering the gene signature further comprises increasing the expression of a transcription factor of the gene.

In some embodiments, the non-lineage committed CD34⁺ cell is a hematopoietic stem and progenitor cell (HSPC). In some embodiments, the step of contacting a population of cells comprising a progenitor cell with a perturbagen causes a change in the cell state. In some embodiments, the erythrocytes are marked by antigen expression CD34+CD38+CD71^{low}+CD235a⁺+CD41⁻. (See Example 2 infra).

In some embodiments, the erythrocytes can be derived from the canonical MEP developmental pathway. In other embodiments, the erythrocytes can be derived from a developmental pathway that does not include the canonical MEP cell. In embodiments, the erythrocytes may be produced from erythropoietin-independent pathway, for example, signal through gp130 and c-kit dramatically promote erythropoiesis from human CD34⁺ cells (Sui et al., Erythropoietin-independent erythrocyte production: signals through gp130 and c-kit dramatically promote erythropoiesis from human CD34⁺ cells, J. Exp. Med., 1996, vol. 183, pp. 837-845).

In some embodiment, the change in the number of proerythroblasts, erythroblasts, reticulocytes, and/or erythrocytes is relative to a control population of cells. For example, in some embodiments, the increase in the number of proerythroblasts, erythroblasts, reticulocytes, and/or erythrocytes upon contacting the cells with a perturbagen—is relative to the population of progenitor cells that is not contacted with the perturbagen. In other embodiments, the increase in the number of proerythroblasts, erythroblasts, reticulocytes, and/or erythrocytes upon contacting the cells with a perturbagen—is relative to the population of progenitor cells prior to contacting it with the perturbagen. In some embodiments, a change in the number of proerythroblasts, erythroblasts, reticulocytes, and/or erythrocytes is caused by change in the state of the cells of a population of progenitor cells. For example, an increase in the number of proerythroblasts, erythroblasts, reticulocytes, and/or erythrocytes within a population of progenitor cell can be due to a change in the state of the cells.

Methods for determining the extension of the lifespan of a specific cell type or a reduction of cell death is well known in the art. For examples, markers for dying cells, e.g., caspases can be detected, or dyes for dead cells, e.g., methylene blue, may be used.

In some embodiments, the number of progenitor cells is decreased. In some embodiments, the decrease in the number of progenitor cells is due in part to decreased cell proliferation of the progenitor cells. In some embodiments, the decrease in the number of progenitor cells is due in part to a decreased lifespan of the progenitor cells. In some embodiments, the decrease in the number of progenitor cells is due in part to increased cell death among the progenitor cells. In some embodiments, the decrease in the number of progenitor cells is relative to the number of progenitor cells in a population of progenitor cells that is not contacted with the at least one perturbagen. In some embodiments, the decrease in the number of progenitor cells is relative to the number of progenitor cells in the population prior to contacting with the at least one perturbagen. In some embodiments, the decrease in the number of progenitor cells is due to a change of cell state from a progenitor cell into the erythrocyte lineage.

In some embodiments, the number of progenitor cells is increased. In some embodiments, the increase in the number of progenitor cells is due in part to increased cell proliferation of the progenitor cells. In some embodiments, the increase in the number of progenitor cells is due in part to an increased lifespan of the progenitor cells. In some embodiments, the increase in the number of progenitor cells is due in part to decreased cell death among the progenitor cells. In some embodiments, the increase in the number of progenitor cells is relative to the number of progenitor cells in a population of progenitor cells that is not contacted with the at least one perturbagen. In some embodiments, the increase in the number of progenitor cells is relative to the number of progenitor cells in the population prior to contacting with the at least one perturbagen.

In some embodiments, the number of proerythroblasts, BFU-E cells, CFU-E cells, early erythroblasts, intermediate erythroblasts, late erythroblasts, reticulocytes, and/or erythrocytes is increased after contacting the population of cells comprising a CD34⁺ cell with the at least one perturbagen. In some embodiments, the number of proerythroblasts, early erythroblasts, intermediate erythroblasts, and/or late erythroblasts is increased after contacting the population of cells comprising a CD34⁺ cell with the at least one perturbagen. In some embodiments, the number of proerythroblasts is increased after contacting the population of cells comprising a CD34⁺ cell with the at least one perturbagen. In other embodiments, the number of proerythroblasts, early erythroblasts, intermediate erythroblasts, and/or late erythroblasts is increased after contacting the population of cells comprising a CD34⁺ cell with the at least one perturbagen. In other embodiments, the number of early erythroblasts, intermediate erythroblasts, and/or late erythroblasts is increased after contacting the population of cells comprising a CD34⁺ cell with the at least one perturbagen. In some embodiments, the number of reticulocytes, and/or erythrocytes is increased after contacting the population of cells comprising a CD34⁺ cell with the at least one perturbagen. In some embodiments, the number of proerythroblasts is increased after contacting the population of cells comprising a CD34⁺ cell with the at least one perturbagen. In some embodiments, the number of proerythroblasts is increased after contacting the population of cells comprising a CD34⁺ cell with the at least one perturbagen. In some embodiments, the number of reticulocytes is increased after contacting the population of cells comprising a CD34⁺ cell with the at least one perturbagen. In some embodiments, the number of erythrocytes is increased after contacting the population of cells comprising a CD34⁺ cell with the at least one perturbagen.

In some embodiments, the ratio of the number of proerythroblasts, erythroblasts, reticulocytes, and/or erythrocytes to the number of progenitor cells is increased relative to the ratio obtained from a population of progenitor cells that is not contacted with the at least one perturbagen. In some embodiments, the ratio of the number of proerythroblasts, erythroblasts, reticulocytes, and/or erythrocytes to the number of progenitor cells is increased relative to the ratio in the population of progenitor cells prior to contacting with the at least one perturbagen.

In some embodiments, the ratio of the number of proerythroblasts, early erythroblasts, intermediate erythroblasts, and/or late erythroblasts to the number of proerythroblasts is increased relative to the ratio obtained from a population of progenitor cells that is not contacted with the at least one perturbagen. In other embodiments, the ratio of the number of proerythroblasts, early erythroblasts, intermediate erythroblasts, and/or late erythroblasts to the number of proerythroblasts is increased relative to the ratio in the population of progenitor cells prior to contacting with the at least one perturbagen.

In some embodiments, the ratio of the number of early erythroblasts, intermediate erythroblasts, and/or late erythroblasts to the number of proerythroblasts is increased relative to the ratio obtained from a population of progenitor cells that is not contacted with the at least one perturbagen. In other embodiments, the ratio of the number of early erythroblasts, intermediate erythroblasts, and/or late erythroblasts to the number of proerythroblasts is increased relative to the ratio in the population of progenitor cells prior to contacting with the at least one perturbagen.

In yet other embodiments, for the methods described herein, the ratio of the number of reticulocytes, and/or erythrocytes to the number of proerythroblasts is increased relative to the ratio obtained from a population of progenitor cells that is not contacted with the at least one perturbagen. In other embodiments, for the methods described here, the ratio of the number of reticulocytes, and/or erythrocytes to the number of proerythroblasts is increased relative to the ratio in the population of progenitor cells prior to contacting with the at least one perturbagen. In yet other embodiments, for the methods described herein, the ratio of the number of reticulocytes to the number of proerythroblasts is increased relative to the ratio obtained from a population of progenitor cells that is not contacted with the at least one perturbagen. In other embodiments, for the methods described here, the ratio of the number of reticulocytes to the number of proerythroblasts is increased relative to the ratio in the population of progenitor cells prior to contacting with the at least one perturbagen. In yet other embodiments, for the methods described herein, the ratio of the number of erythrocytes to the number of proerythroblasts is increased relative to the ratio obtained from a population of progenitor cells that is not contacted with the at least one perturbagen. In other embodiments, for the methods described here, the ratio of the number of erythrocytes to the number of proerythroblasts is increased relative to the ratio in the population of progenitor cells prior to contacting with the at least one perturbagen.

In some embodiments, for the methods described herein, the ratio of the number of reticulocytes and/or erythrocytes to the number of proerythroblasts, early erythroblasts, intermediate erythroblasts, and/or late erythroblasts is increased relative to the ratio obtained from a population of progenitor cells that is not contacted with the at least one perturbagen. In other embodiments, for the methods described herein, the ratio of the number of reticulocytes and/or erythrocytes to the number of proerythroblasts, early erythroblasts, intermediate erythroblasts, and/or late erythroblasts is increased relative to the ratio in the population of progenitor cells prior to contacting with the at least one perturbagen. In some embodiments, for the methods described herein, the ratio of the number of erythrocytes to the number of proerythroblasts, early erythroblasts, intermediate erythroblasts, and/or late erythroblasts is increased relative to the ratio obtained from a population of progenitor cells that is not contacted with the at least one perturbagen. In other embodiments, for the methods described herein, the ratio of the number of erythrocytes to the number of proerythroblasts, early erythroblasts, intermediate erythroblasts, and/or late erythroblasts is increased relative to the ratio in the population of progenitor cells prior to contacting with the at least one perturbagen. In some embodiments, for the methods described herein, the ratio of the number of reticulocytes to the number of proerythroblasts, early erythroblasts, intermediate erythroblasts, and/or late erythroblasts is increased relative to the ratio obtained from a population of progenitor cells that is not contacted with the at least one perturbagen. In other embodiments, for the methods described herein, the ratio of the number of reticulocytes to the number of proerythroblasts, early erythroblasts, intermediate erythroblasts, and/or late erythroblasts is increased relative to the ratio in the population of progenitor cells prior to contacting with the at least one perturbagen.

In some embodiments, for the methods described herein, the ratio of the number of reticulocytes, and/or erythrocytes to the number of early erythroblasts, intermediate erythroblasts, and/or late erythroblasts is increased relative to the ratio obtained from a population of progenitor cells that is not contacted with the at least one perturbagen. In other embodiments, for the methods described here, the ratio of the number of reticulocytes, and/or erythrocytes to the number of early erythroblasts, intermediate erythroblasts, and/or late erythroblasts is increased relative to the ratio in the population of progenitor cells prior to contacting with the at least one perturbagen. In some embodiments, for the methods described herein, the ratio of the number of reticulocytes to the number of early erythroblasts, intermediate erythroblasts, and/or late erythroblasts is increased relative to the ratio obtained from a population of progenitor cells that is not contacted with the at least one perturbagen. In other embodiments, for the methods described here, the ratio of the number of reticulocytes to the number of early erythroblasts, intermediate erythroblasts, and/or late erythroblasts is increased relative to the ratio in the population of progenitor cells prior to contacting with the at least one perturbagen. In some embodiments, the ratio of the number erythrocytes to the number of early erythroblasts, intermediate erythroblasts, and/or late erythroblasts is increased relative to the ratio obtained from a population of progenitor cells that is not contacted with the at least one perturbagen. In other embodiments, the ratio of the number of erythrocytes to the number of early erythroblasts, intermediate erythroblasts, and/or late erythroblasts is increased relative to the ratio in the population of progenitor cells prior to contacting with the at least one perturbagen.

In some embodiments, the ratio of the number of proerythroblasts, early erythroblasts, intermediate erythroblasts, and/or late erythroblasts to the number of proerythroblasts is increased relative to the ratio obtained from a population of progenitor cells that is not contacted with the at least one perturbagen. In other embodiments, the ratio of the number of proerythroblasts, early erythroblasts, intermediate erythroblasts, and/or late erythroblasts to the number of proerythroblasts is increased relative to the ratio in the population of progenitor cells prior to contacting with the at least one perturbagen. In some embodiments, the ratio of the number of proerythroblasts to the number of proerythroblasts is increased relative to the ratio obtained from a population of progenitor cells that is not contacted with the at least one perturbagen. In other embodiments, the ratio of the number of proerythroblasts to the number of proerythroblasts is increased relative to the ratio in the population of progenitor cells prior to contacting with the at least one perturbagen. In some embodiments, the ratio of the number of early erythroblasts, intermediate erythroblasts, and/or late erythroblasts to the number of proerythroblasts is increased relative to the ratio obtained from a population of progenitor cells that is not contacted with the at least one perturbagen. In other embodiments, the ratio of the number of early erythroblasts, intermediate erythroblasts, and/or late erythroblasts to the number of proerythroblasts is increased relative to the ratio in the population of progenitor cells prior to contacting with the at least one perturbagen.

In some embodiments, the ratio of the number of early erythroblasts, intermediate erythroblasts, late erythroblasts, reticulocytes, and/or erythrocytes to the number of proerythroblasts is increased relative to the ratio obtained from a population of progenitor cells that is not contacted with the at least one perturbagen. In other embodiments, the ratio of the number of early erythroblasts, intermediate erythroblasts, late erythroblasts, reticulocytes, and/or erythrocytes to the number of proerythroblasts is increased relative to the ratio in the population of progenitor cells prior to contacting with the at least one perturbagen.

In some embodiments, the ratio of the number of early erythroblasts, intermediate erythroblasts, late erythroblasts, reticulocytes, and/or erythrocytes to the number of proerythroblasts is increased relative to the ratio obtained from a population of progenitor cells that is not contacted with the at least one perturbagen. In other embodiments, the ratio of the number of early erythroblasts, intermediate erythroblasts, late erythroblasts, reticulocytes, and/or erythrocytes to the number of proerythroblasts is increased relative to the ratio in the population of progenitor cells prior to contacting with the at least one perturbagen.

In some embodiments, the ratio of the number of reticulocytes, and/or erythrocytes to the number of proerythroblasts is increased relative to the ratio obtained from a population of progenitor cells that is not contacted with the at least one perturbagen. In other embodiments, the ratio of the number of reticulocytes, and/or erythrocytes to the number of proerythroblasts is increased relative to the ratio in the population of progenitor cells prior to contacting with the at least one perturbagen. In some embodiments, the ratio of the number of reticulocytes to the number of proerythroblasts is increased relative to the ratio obtained from a population of progenitor cells that is not contacted with the at least one perturbagen. In other embodiments, the ratio of the number of reticulocytes to the number of proerythroblasts is increased relative to the ratio in the population of progenitor cells prior to contacting with the at least one perturbagen. In some embodiments, the ratio of the number of erythrocytes to the number of proerythroblasts is increased relative to the ratio obtained from a population of progenitor cells that is not contacted with the at least one perturbagen. In other embodiments, the ratio of the number of erythrocytes to the number of proerythroblasts is increased relative to the ratio in the population of progenitor cells prior to contacting with the at least one perturbagen.

In some embodiments of the methods described herein, the ratio of the number of erythrocytes to the number of erythroblasts and/or reticulocytes is increased relative to the ratio obtained from a population of progenitor cells that is not contacted with the at least one perturbagen. In other embodiments, the ratio of the number of erythrocytes to the number of erythroblasts and/or reticulocytes is increased relative to the ratio in the population of progenitor cells prior to contacting with the at least one perturbagen.

In some embodiments, the number of proerythroblasts is decreased. In some embodiments, the number of erythroblasts is decreased. In some embodiments, the number of proerythroblasts is decreased. In some embodiments, the number of proerythroblasts is increased. In some embodiments, the number of early erythroblasts, intermediate erythroblasts, and/or late erythroblasts is increased. In some embodiments, the number of reticulocytes is increased. In some embodiments, the number of proerythroblasts is increased.

Methods for counting cells are well known in the art. Non-limiting examples include hemocytometry, flow cytometry, and cell sorting techniques, e.g., fluorescence activated cell sorting (FACS). The maturation of the erythrocytes is, in some embodiments, determined by loss of CD71 expression. Erythroid maturation is determined by, in some embodiments, flow cytometry using a four-antibody panel (See Example 2 infra) (CD71, CD235a, CD233, CD49d) with increased CD233 expression, with a concomitant loss of CD49d expression, and a shift in CD71^{Hi} to CD71^{low} erythroid population (CD235a+).

In some embodiments, the change in cell state of a progenitor cell provides an increase in the number of one or more of proerythroblasts, early erythroblasts, intermediate erythroblasts, late erythroblasts, reticulocytes, and erythrocytes. In some embodiments, one or more of the proerythroblasts, early erythroblasts, intermediate erythroblasts, late erythroblasts, reticulocytes, and erythrocytes comprise fetal hemoglobin (HbF).

In one embodiment, the change in cell state of a progenitor cell provides an increase in F cells. See Boyer et al. 1975. Fetal hemoglobin restriction to a few erythrocytes (F cells) in normal human adults. Science 188: 361-363

In some embodiments, one or more of the proerythroblasts, early erythroblasts, intermediate erythroblasts, late erythroblasts, reticulocytes, and erythrocytes expresses HBG1 and/or HBG2. In embodiments, the increase in the number of erythrocytes comprising HbF is relative to the number of erythrocytes obtained from a population of progenitor cells that is not contacted with the at least one perturbagen. In other embodiments, the increase in the number of erythrocytes comprising HbF is relative to the population of progenitor cells prior to contacting with the at least one perturbagen.

Methods for counting cells are well known in the art. Non-limiting examples include hemocytometry, flow cytometry, and cell sorting techniques, e.g., fluorescence activated cell sorting (FACS). The maturation of the erythrocytes is, in some embodiments, determined by loss of CD71 expression. Erythroid maturation is determined by, in some embodiments, flow cytometry using a four-antibody panel (See Example 2 infra) (CD71, CD235a, CD233, CD49d) with increased CD233 expression, with a concomitant loss of CD49d expression, and a shift in CD71^{Hi} to CD71^{low} erythroid population (CD235a+).

In some embodiments, the change in cell state of a progenitor cell provides an increase in the number of one or more of proerythroblasts, early erythroblasts, intermediate erythroblasts, late erythroblasts, reticulocytes, and erythrocytes. In this aspect, one or more of the proerythroblasts, early erythroblasts, intermediate erythroblasts, late erythroblasts, reticulocytes, and erythrocytes comprise fetal hemoglobin (HbF). In some embodiments, one or more of the proerythroblasts, early erythroblasts, intermediate erythroblasts, late erythroblasts, reticulocytes, and erythrocytes expresses HBG1 and/or HBG2.

In an embodiment, the change in cell state of a progenitor cell provides an increase in F cells. See Boyer et al. 1975. Fetal hemoglobin restriction to a few erythrocytes (F cells) in normal human adults. Science 188: 361-363

In some embodiments, the increase in the number of erythrocytes comprising HbF is relative to the number of erythrocytes obtained from a population of progenitor cells that is not contacted with the at least one perturbagen. In other embodiments, the increase in the number of erythrocytes comprising HbF is relative to the population of progenitor cells prior to contacting with the at least one perturbagen.

In some embodiments, the change in cell state of a progenitor cell provides an increase in the number of erythrocytes comprising HbF.

In an embodiment, the change in cell state provides an increase in the ratio of the number of erythrocytes comprising HbF to the number of progenitor cells relative to the ratio obtained from a population of progenitor cells that is not contacted with the at least one perturbagen. In an embodiment, the change in cell state provides an increase in the ratio of the number of erythrocytes comprising HbF to the number of progenitor cells relative to the ratio in the population of progenitor cells prior to contacting with the at least one perturbagen.

In an embodiment, the increase in the number of erythrocytes comprising HbF, is due in part to increased cell proliferation of the erythrocytes comprising HbF. In other embodiments, the increase in the number of erythrocytes comprising HbF, is due in part to an increased lifespan of the erythrocytes comprising HbF. In other embodiments, the increase in the number of erythrocytes comprising HbF, is due in part to reduced cell death among the erythrocytes comprising HbF. In other embodiments, the increase in the number of erythrocytes comprising HbF, is due in part to a change of cell state from progenitor cells into the erythrocyte lineage.

In an embodiment, the change in cell state provides a decrease in the number of progenitor cells. In embodiments, the decrease in the number of progenitor cells is due in part to decreased cell proliferation of the progenitor cells. In other embodiments, the decrease in the number of progenitor cells is due in part to a decreased lifespan of the progenitor cells. In other embodiments, the decrease in the number of progenitor cells is due in part to increased cell death among the progenitor cells. In some embodiments, the decrease in the number of progenitor cells is relative to the number of progenitor cells in a population of progenitor cells that is not contacted with the at least one perturbagen. In some embodiments, the decrease in the number of progenitor cells is relative to the number of progenitor cells in the population prior to contacting with the at least one perturbagen. In some embodiments, the decrease in the number of progenitor cells is due to a change of cell state from a progenitor cell into the erythrocyte lineage.

In an embodiment, the change in cell state provides an increase in the number of progenitor cells. In embodiments, the increase in the number of progenitor cells is due in part to increased cell proliferation of the progenitor cells. In other embodiments, the increase in the number of progenitor cells is due in part to an increased lifespan of the progenitor cells. In other embodiments, the increase in the number of progenitor cells is due in part to decreased cell death among the progenitor cells. In some embodiments, the increase in the number of progenitor cells is relative to the number of progenitor cells in a population of progenitor cells that is not contacted with the at least one perturbagen. In some embodiments, the increase in the number of progenitor cells is relative to the number of progenitor cells in the population prior to contacting with the at least one perturbagen.

In an embodiment, the change in cell state provides an increase in the number of proerythroblasts, BFU-E cells, CFU-E cells, early erythroblasts, intermediate erythroblasts, late erythroblasts, reticulocytes, and/or erythrocytes after contacting the population of cells comprising a CD34⁺ cell with the at least one perturbagen. In embodiments, one or more of the proerythroblasts, early erythroblasts, intermediate erythroblasts, late erythroblasts, reticulocytes, and erythrocytes comprise HbF.

In an embodiment, the change in cell state provides an increase in the ratio of the number of other committed blood cells to the number of progenitor cells relative to the ratio obtained from a population of progenitor cells that is not contacted with the at least one perturbagen.

In an embodiment, the change in cell state provides an increase in the ratio of the number of proerythroblasts, BFU-E cells, CFU-E cells, early erythroblasts, intermediate erythroblasts, late erythroblasts, reticulocytes, and/or erythrocytes to the number of progenitor cells relative to the ratio obtained from a population of progenitor cells that is not contacted with the at least one perturbagen. In embodiments, one or more of the proerythroblasts, early erythroblasts, intermediate erythroblasts, late erythroblasts, reticulocytes, and erythrocytes comprise HbF. In other embodiments, the ratio of the number proerythroblasts, BFU-E cells, CFU-E cells, early erythroblasts, intermediate erythroblasts, late erythroblasts, reticulocytes, and/or erythrocytes to the number of progenitor cells is increased relative to the ratio in the population of progenitor cells prior to contacting with the at least one perturbagen. In embodiments, one or more of the proerythroblasts, early erythroblasts, intermediate erythroblasts, late erythroblasts, reticulocytes, and erythrocytes comprise HbF.

In some embodiments, the change in cell state provides an increase in the number of proerythroblasts and/or erythrocytes comprising HbF after contacting the population of cells comprising a CD34⁺ cell with the at least one perturbagen. In other embodiments, the number of reticulocytes comprising HbF, and/or erythrocytes comprising HbF is increased after contacting the population of cells comprising a CD34⁺ cell with the at least one perturbagen.

In some embodiments, the change in cell state provides an increase in the ratio of the number of early erythroblasts, intermediate erythroblasts, late erythroblasts, reticulocytes, and/or erythrocytes to the number of proerythroblasts relative to the ratio obtained from a population of progenitor cells that is not contacted with the at least one perturbagen. In embodiments, one or more of the proerythroblasts, early erythroblasts, intermediate erythroblasts, late erythroblasts, reticulocytes, and erythrocytes comprise HbF.

In some embodiments, the change in cell state provides an increase in the ratio of the number of HbF-expressing early erythroblasts, intermediate erythroblasts, and/or late erythroblasts to the number of HbF-expressing proerythroblasts relative to the ratio obtained from a population of progenitor cells that is not contacted with the at least one perturbagen. In other embodiments, the ratio of the number of HbF-expressing early erythroblasts, intermediate erythroblasts, and/or late erythroblasts to the number of HbF-expressing proerythroblasts is increased relative to the ratio in the population of progenitor cells prior to contacting with the at least one perturbagen.

In other embodiments, the ratio of the number of early erythroblasts, intermediate erythroblasts, late erythroblasts, reticulocytes, and/or erythrocytes to the number of proerythroblasts is increased relative to the ratio in the population of progenitor cells prior to contacting with the at least one perturbagen. In embodiments, one or more of the proerythroblasts, early erythroblasts, intermediate erythroblasts, late erythroblasts, reticulocytes, and erythrocytes comprise HbF.

In some embodiments, the change in cell state provides an increase in the ratio of the number of HbF-expressing early erythroblasts to the number of HbF-expressing proerythroblasts relative to the ratio obtained from a population of progenitor cells that is not contacted with the at least one perturbagen. In other embodiments, the ratio of the number of HbF-expressing early erythroblasts to the number of HbF-expressing proerythroblasts is increased relative to the ratio in the population of progenitor cells prior to contacting with the at least one perturbagen.

In some embodiments, the change in cell state provides an increase in the ratio of the number of HbF-expressing intermediate erythroblasts to the number of HbF-expressing early erythroblasts relative to the ratio obtained from a population of progenitor cells that is not contacted with the at least one perturbagen. In other embodiments, the ratio of the number of HbF-expressing intermediate erythroblasts to the number of HbF-expressing early erythroblasts is increased relative to the ratio in the population of progenitor cells prior to contacting with the at least one perturbagen.

In some embodiments, the change in cell state provides an increase in the ratio of the number of HbF-expressing late erythroblasts to the number of HbF-expressing intermediate erythroblasts relative to the ratio obtained from a population of progenitor cells that is not contacted with the at least one perturbagen. In other embodiments, the ratio of the number of HbF-expressing late erythroblasts to the number of HbF-expressing intermediate erythroblasts is increased relative to the ratio in the population of progenitor cells prior to contacting with the at least one perturbagen.

In some embodiments, the change in cell state provides an increase in the ratio of the number of reticulocytes comprising HbF to the number of HbF-expressing late erythroblasts relative to the ratio obtained from a population of progenitor cells that is not contacted with the at least one perturbagen. In other embodiments, the ratio of the number of reticulocytes comprising HbF to the number of HbF-expressing late erythroblasts is increased relative to the ratio in the population of progenitor cells prior to contacting with the at least one perturbagen.

In some embodiments, the change in cell state provides an increase in the ratio of the number of erythrocytes comprising HbF to the number of reticulocytes comprising HbF relative to the ratio obtained from a population of progenitor cells that is not contacted with the at least one perturbagen. In other embodiments, the ratio of the number of erythrocytes comprising HbF to the number of reticulocytes comprising HbF is increased relative to the ratio in the population of progenitor cells prior to contacting with the at least one perturbagen.

In some embodiments, the change in cell state provides an increase in the ratio of the number of erythrocytes comprising HbF to HbF-expressing late erythroblasts relative to the ratio obtained from a population of progenitor cells that is not contacted with the at least one perturbagen. In other embodiments, the ratio of the number of erythrocytes comprising HbF to HbF-expressing late erythroblasts is increased relative to the ratio in the population of progenitor cells prior to contacting with the at least one perturbagen.

In some embodiments, the change in cell state provides an increase in the ratio of the number of erythrocytes comprising HbF to HbF-expressing intermediate erythroblasts relative to the ratio obtained from a population of progenitor cells that is not contacted with the at least one perturbagen. In other embodiments, the ratio of the number of erythrocytes comprising HbF to HbF-expressing intermediate erythroblasts is increased relative to the ratio in the population of progenitor cells prior to contacting with the at least one perturbagen.

In some embodiments, the change in cell state provides an increase in the ratio of the number of erythrocytes comprising HbF to HbF-expressing early erythroblasts relative to the ratio obtained from a population of progenitor cells that is not contacted with the at least one perturbagen. In other embodiments, the ratio of the number of erythrocytes comprising HbF to HbF-expressing early erythroblasts is increased relative to the ratio in the population of progenitor cells prior to contacting with the at least one perturbagen.

In some embodiments, the change in cell state provides an increase in the ratio of the number of erythrocytes comprising HbF to HbF-expressing proerythroblasts relative to the ratio obtained from a population of progenitor cells that is not contacted with the at least one perturbagen. In other embodiments, the ratio of the number of erythrocytes comprising HbF to HbF-expressing proerythroblasts is increased relative to the ratio in the population of progenitor cells prior to contacting with the at least one perturbagen.

In some embodiments, the change in cell state provides an increase in the ratio of the number of erythrocytes comprising HbF to proerythroblasts relative to the ratio obtained from a population of progenitor cells that is not contacted with the at least one perturbagen. In other embodiments, the ratio of the number of erythrocytes comprising HbF to proerythroblasts is increased relative to the ratio in the population of progenitor cells prior to contacting with the at least one perturbagen.

In some embodiments, the change in cell state provides an increase in the ratio of the number of erythrocytes comprising HbF and/or reticulocytes to the number of HbF-expressing early erythroblasts, intermediate erythroblasts, and/or late erythroblasts relative to the ratio obtained from a population of progenitor cells that is not contacted with the at least one perturbagen. In other embodiments, the ratio of the number of erythrocytes comprising HbF and/or reticulocytes to the number of HbF-expressing early erythroblasts, intermediate erythroblasts, and/or late erythroblasts is increased relative to the ratio in the population of progenitor cells prior to contacting with the at least one perturbagen.

In some embodiments, the change in cell state provides an increase in the ratio of the number of erythrocytes comprising HbF to the number of HbF-expressing early erythroblasts, intermediate erythroblasts, and/or late erythroblasts relative to the ratio obtained from a population of progenitor cells that is not contacted with the at least one perturbagen. In other embodiments, the ratio of the number of erythrocytes comprising HbF to the number of HbF-expressing early erythroblasts, intermediate erythroblasts, and/or late erythroblasts is increased relative to the ratio in the population of progenitor cells prior to contacting with the at least one perturbagen. In some embodiments, the change in cell state provides an increase in the ratio of the number of reticulocytes comprising HbF to the number of HbF-expressing early erythroblasts, intermediate erythroblasts, and/or late erythroblasts relative to the ratio obtained from a population of progenitor cells that is not contacted with the at least one perturbagen. In other embodiments, the ratio of the number of reticulocytes comprising HbF to the number of HbF-expressing early erythroblasts, intermediate erythroblasts, and/or late erythroblasts is increased relative to the ratio in the population of progenitor cells prior to contacting with the at least one perturbagen.

In some embodiments, the change in cell state provides an increase in the ratio of the number of erythrocytes comprising HbF to reticulocytes comprising HbF relative to the ratio obtained from a population of progenitor cells that is not contacted with the at least one perturbagen. In other embodiments, the ratio of the number of erythrocytes comprising HbF to reticulocytes comprising HbF is increased relative to the ratio in the population of progenitor cells prior to contacting with the at least one perturbagen.

In one embodiment, the change in cell state provides a decrease in the number of proerythroblasts. In another embodiment, the change in cell state provides a decrease in the number of HbF-negative or HbF-low proerythroblasts. In another embodiment, the number of HbF-negative or HbF-low early erythroblasts is decreased. In another embodiment, the number of HbF-negative or HbF-low intermediate erythroblasts is decreased. In another embodiment, the number of HbF-negative or HbF-low late erythroblasts is decreased. In another embodiment, the number of HbF-negative or HbF-low reticulocytes is decreased.

In one embodiment, the change in cell state provides an increase in the number of proerythroblasts. In another embodiment, the number of HbF-positive or HbF-high proerythroblasts is increased. In another embodiment, the number of HbF-positive or HbF-high early erythroblasts is increased. In another embodiment, the number of HbF-positive or HbF-high intermediate erythroblasts is increased. In another embodiment, the number of HbF-positive or HbF-high late erythroblasts is increased. In another embodiment, the number of HbF-positive or HbF-high reticulocytes is increased. In another embodiment, the number of HbF-positive or HbF-high erythrocytes is increased.

In an embodiment, the change in cell state provides an increase in the number of F cells. In another embodiment, the ratio of the number of F cells to non-F cells is increased relative to the ratio obtained from a population of progenitor cells that is not contacted with the at least one perturbagen. In another embodiment, the ratio of the number of F cells to non-F cells is increased relative to the ratio in the population of progenitor cells prior to contacting with the at least one perturbagen.

In some embodiments, contacting the population of progenitor cells with the at least one perturbagen occurs in vitro or ex vivo. In an embodiment, contacting the population of progenitor cells with the at least one perturbagen occurs in vivo in a subject. In an embodiment, the subject is a human. In some embodiments, the human is an adult human. In some embodiments, the adult human has an abnormal number of one or more of erythroblasts, or erythrocytes, or a disease or disorder characterized thereby. In some embodiments, the adult human has a disease or disorder characterized by abnormal oxygen delivery, or hemoglobin deficiency. In some embodiments, the adult human suffers from a sickle cell disease or a thalassemia.

Percentage of fetal hemoglobin (% HbF) and fetal hemoglobin containing red blood cells (% F^{+ cells) are important parameters for determining the efficacy of the perturbagens as described above at promoting a fetal hemoglobin (HbF) cell state. % HbF is the proportion of HbF in the total Hb in hemolysate, which ignores the numbers of red blood cells. % F}⁺ cells is the proportion of the HbF containing red blood cells in total red blood cells. (See Mundee et al., Cytometry (Communication in Clinical Cytometry), 2000, vol. 42, p. 389-393).

Methods for assaying % HbF and % F⁺ cells are well known in the art. Non-limiting examples include high-performance liquid chromatography (HPLC), flow cytometry, or ion-exchange chromatography. The HbF % is usually measured by HPLC. The flow cytometry assay, the standard clinical method, may be used for assaying % F⁺ cells by immunofluorescent techniques. In addition to flow cytometry, ion-exchange chromatography may be used to measure the fraction HbF relative to all other hemoglobin (HbF/HbA+HbF) (See Example 2 below).

The baseline level of HbF and F cells in the total blood may serve as control for determining the efficacy of the perturbagens upon induction of HbF in red blood cells. Non-limiting examples of HbF level in a subject may serve as baseline % HbF include DMSO negative control, a normal individual, a normal individual of specific ethnicity, an individual having sickle cell disease, an individual having sickle cell disease with hydroxyurea treatment, or a population of erythrocytes having specific % HbF etc.

The mean % HbF and % F⁺ cells in normal adults is 1.0%±0.1% and 3.0%±0.4%. The normal adult is considered to have very low % HbF and % F⁺ cells. The mean % HbF of about 3.0% or less is considered as low % HbF. The mean % HbF ranging from about 4.0% to 12.0% is considered as medium % HbF, for example, mean % HbF and % F⁺ cells in sickle cell patient is 4.1%±0.8% and 14.8%±1.8%. The mean % HbF of about 13.0% or higher is considered as high % HbF, e.g., the mean % HbF and % F⁺ cells in sickle cell patient treated with hydroxyurea is 15.8%±2.0% and 54.1%±8.5%. (See Mundee et al. supra). In embodiments, % HbF is measured by HPLC. In embodiments, % F+ cells are measured by flow cytometry.

In various embodiments, the baseline % HbF and induced % HbF is gated by vehicle control and % HbF induced by 50 NM hydroxyurea (Example 2 infra). It is to be understood that the HbF-low and HbF-high F⁺ cells characterization described above are classified by the mean % HbF values that typically presents in normal adult, patient having sickle cell disease and sickle cell disease patent with hydroxyurea treatment. It is to be understood that the absolute value ranges for mean % HbF that characterized as low and high % HbF may vary depends on the assay techniques and detection limits thereof.

In one aspect, the disclosure provides a population comprising about 20% or more, about 25% or more, about 30% or more, about 35% or more, about 40% or more, about 45% or more, about 50% or more, about 55% or more, or about 60% or more of F⁺ cells, wherein the population has not been subjected to a step of cell fractionation. Non-limiting examples of cell fractionation include selection (e.g. positive or negative) methods, such as fluorescence-activated cell sorting (FACS), magnetic-based cell separation, immunomagnetic-based cell separation, density gradient centrifugation, immunodensity cell separation, sedimentation, adhesion, microfluidic methods, and the like.

In one aspect of the present disclosure is related to a method for inducing a cell state of a progenitor cell. In some embodiments, the cell state is the (CD34+/CD41^{Low}+/CD235a+) cell state. In embodiments, this method includes a step of contacting a population of cells comprising a progenitor cell with at least one perturbagen selected from Table 3, or a variant of perturbagens described in Table 3. In one embodiment, the progenitor cell is a non-lineage committed CD34+ cell. In one embodiment, the progenitor cell is a non-lineage committed CD34+ cell. In one embodiment, the progenitor cell is a non-lineage committed CD34+ cells selected from a hematopoietic stem cell (an HSC; e.g., a CD34+ HSC), a burst-forming unit-erythroid (BFU-E) cell, a colony forming unit-erythroid (CFU-E) cell, a proerythroblast, a basophilic erythroblast (also known as an early erythroblast), a polychromatic erythroblast (also known as an intermediate erythroblast), and a orthochromatic erythroblast (also known as a late erythroblasts). In some embodiments, the method includes increasing the expression of the glucocorticoid receptor (NR3C1) and/or the C-Kit receptor.

In one aspect of the present disclosure is related to a method for inducing a cell state of a progenitor cell. In some embodiments, the cell state is the (CD34+/CD41^{Low}+/CD235a+) cell state. This method includes a step of contacting a population of cells comprising a progenitor cell with at least one perturbagen selected from Table 3, or a variant of perturbagens described in Table 3, and capable of altering a gene signature in the progenitor cell. In some embodiments, altering the gene signature comprises an increase in expression and/or activity in the progenitor cell of one or more genes selected from the genes designated as an “up” gene in the gene directionality column of Table 1a and/or Table 2 and/or a decrease in expression and/or activity in the progenitor cell of one or more genes selected from the genes designated as a “down” gene in the gene directionality column of Table 1a and/or Table 2 and/or an increase in expression and/or activity in the progenitor cell of one or more genes selected from Table 1b and/or a decrease in expression and/or activity in the progenitor cell of one or more genes selected from Table 1b. In one embodiment, the progenitor cell is a non-lineage committed CD34+ cell. In one embodiment, the progenitor cell is a non-lineage committed CD34⁺ cell. In one embodiment, the progenitor cell is a non-lineage committed CD34⁺ cells selected from a hematopoietic stem cell (an HSC; e.g., a CD34+ HSC), a burst-forming unit-erythroid (BFU-E) cell, a colony forming unit-erythroid (CFU-E) cell, a proerythroblast, a basophilic erythroblast (also known as an early erythroblast), a polychromatic erythroblast (also known as an intermediate erythroblast), and a orthochromatic erythroblast (also known as a late erythroblasts). In some embodiments, the method includes increasing the expression of the glucocorticoid receptor (NR3C1) and/or the C-Kit receptor.

In one aspect of the present disclosure is related to a method for inducing a cell state of a progenitor cell. In some embodiments, the cell state is the (CD34+/CD41^{Low}+/CD235a+) cell state. In embodiments, this method includes a step of contacting a population of cells comprising a progenitor cell with a combination of hydroxyurea (HU) and at least one perturbagen selected from Table 3, or a variant of perturbagens described in Table 3, or a variant of perturbagens described in Table 3. In one embodiment, the progenitor cell is a non-lineage committed CD34⁺ cell. In one embodiment, the progenitor cell is a non-lineage committed CD34⁺ cell. In one embodiment, the progenitor cell is a non-lineage committed CD34⁺ cells selected from a hematopoietic stem cell (an HSC; e.g., a CD34+ HSC), a burst-forming unit-erythroid (BFU-E) cell, a colony forming unit-erythroid (CFU-E) cell, a proerythroblast, a basophilic erythroblast (also known as an early erythroblast), a polychromatic erythroblast (also known as an intermediate erythroblast), and a orthochromatic erythroblast (also known as a late erythroblasts). In some embodiments, the method includes increasing the expression of the glucocorticoid receptor (NR3C1) and/or the C-Kit receptor.

In one aspect of the present disclosure is related to a method for inducing a cell state of a progenitor cell. In some embodiments, the cell state is the (CD34+/CD41^{Low}+/CD235a+) cell state. This method includes a step of contacting a population of cells comprising a progenitor cell with a combination of hydroxyurea (HU) and at least one perturbagen selected from Table 3, or a variant of perturbagens described in Table 3, and capable of altering a gene signature in the progenitor cell. In some embodiments, altering the gene signature comprises an increase in expression and/or activity in the progenitor cell of one or more genes selected from the genes designated as an “up” gene in the gene directionality column of Table 1a and/or Table 2 and/or a decrease in expression and/or activity in the progenitor cell of one or more genes selected from the genes designated as a “down” gene in the gene directionality column of Table 1a and/or Table 2 and/or an increase in expression and/or activity in the progenitor cell of one or more genes selected from Table 1b and/or a decrease in expression and/or activity in the progenitor cell of one or more genes selected from Table 1b. In one embodiment, the progenitor cell is a non-lineage committed CD34⁺ cell. In one embodiment, the progenitor cell is a non-lineage committed CD34⁺ cell. In one embodiment, the progenitor cell is a non-lineage committed CD34⁺ cells selected from a hematopoietic stem cell (an HSC; e.g., a CD34+ HSC), a burst-forming unit-erythroid (BFU-E) cell, a colony forming unit-erythroid (CFU-E) cell, a proerythroblast, a basophilic erythroblast (also known as an early erythroblast), a polychromatic erythroblast (also known as an intermediate erythroblast), and a orthochromatic erythroblast (also known as a late erythroblasts). In some embodiments, the method includes increasing the expression of the glucocorticoid receptor (NR3C1) and/or the C-Kit receptor.

In one aspect of the present disclosure is related to a method for inducing a cell state of a progenitor cell. In some embodiments, the cell state is the (CD34+/CD41^{Low}+/CD235a+) cell state. In embodiments, this method includes a step of contacting a population of cells comprising a progenitor cell with a combination of hydroxyurea (HU) and at least one glucocorticoid. In one embodiment, the progenitor cell is a non-lineage committed CD34⁺ cell. In one embodiment, the progenitor cell is a non-lineage committed CD34⁺ cell. In one embodiment, the progenitor cell is a non-lineage committed CD34⁺ cells selected from a hematopoietic stem cell (an HSC; e.g., a CD34+ HSC), a burst-forming unit-erythroid (BFU-E) cell, a colony forming unit-erythroid (CFU-E) cell, a proerythroblast, a basophilic erythroblast (also known as an early erythroblast), a polychromatic erythroblast (also known as an intermediate erythroblast), and a orthochromatic erythroblast (also known as a late erythroblasts). In some embodiments, the method includes increasing the expression of the glucocorticoid receptor (NR3C1) and/or the C-Kit receptor. In some embodiments, the glucocorticoid is dexamethasone.

In one aspect the present disclosure is related to a method for inducing a cell state of a progenitor cell. In some embodiments, the cell state is the (CD34+/CD41^{Low}+/CD235a+) cell state. This method includes a step of contacting a population of cells comprising a progenitor cell with a combination of hydroxyurea (HU) and at least one glucocorticoid, and capable of altering a gene signature in the progenitor cell. In one embodiment, the progenitor cell is a non-lineage committed CD34⁺ cell. In one embodiment, the progenitor cell is a non-lineage committed CD34⁺ cell. In one embodiment, the progenitor cell is a non-lineage committed CD34⁺ cells selected from a hematopoietic stem cell (an HSC; e.g., a CD34+ HSC), a burst-forming unit-erythroid (BFU-E) cell, a colony forming unit-erythroid (CFU-E) cell, a proerythroblast, a basophilic erythroblast (also known as an early erythroblast), a polychromatic erythroblast (also known as an intermediate erythroblast), and a orthochromatic erythroblast (also known as a late erythroblasts). In some embodiments, the method includes increasing the expression of the glucocorticoid receptor (NR3C1) and/or the C-Kit receptor. In some embodiments, the glucocorticoid is dexamethasone.

In one aspect, inducing a cell state of a progenitor cell using the methods of the disclosure provides isolated populations of cells having the (CD34+/CD41^{Low}+/CD235a+) cell state. In some embodiments, about 20% or more, about 25% or more, about 30% or more, about 35% or more, about 40% or more, about 45% or more, about 50% or more, about 55% or more, or about 60% or more of the population of cells comprise the (CD34+/CD41^{Low}+/CD235a+) cell state.

In some embodiments, the at least one perturbagen selected from Table 3, or a variant thereof, comprises at least 2, at least 3, at least 4, or at least 5 perturbagens selected from Table 3, or variants thereof.

In some embodiments, altering the gene signature comprises increased expression and/or increased activity in the progenitor cell of one or more genes selected from the genes designated as an “up” gene in the gene directionality column of Table 1a. In some embodiments, the one or more genes selected from the genes designated as an “up” gene in the gene directionality column of Table 1a comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, 81 or more, 82 or more, 83 or more, 84 or more, 85 or more, 86 or more, 87 or more, 88 or more, 89 or more, 90 or more, 91 or more, 92 or more, 93 or more, 94 or more, 95 or more, 96 or more, 97 or more, 98 or more, 99 or more, 100 or more, 101 or more, 102 or more, 103 or more, 104 or more, or 105 or more genes. In another embodiment, the one or more genes selected from the genes designated as an “up” gene in the gene directionality column of Table 1a comprises at least one of DDIT4, EPRS, MTHFD2, EIF4EBP1, AARS, ABCC5, PHGDH, TUBB6, LSM6, EIF4G1, RNF167, CD320, CTNNAL1, GADD45A, PTK2, CFLAR, IGF2BP2, CDK1, CDC45, CDCA4, MELK, HAT1, PAK1, TSPAN6, TIMM17B, KDM5A, UBE3B, RPS5, PAICS, RPIA, KDELR2, PNP, CAST, H2AFV, ATP11B, CTNND1, ORC1, FDFT1, CDKN1B, INSIG1, IGF1R, TRAP1, TSTA3, SUZ12, CDK4, HMGCS1, LAP3, TBPL1, FAH, CCP110, APOE, IGF2R, DYRK3, MYBL2, APP, DNMT1, SMC3, HTATSF1, CAT, ACAT2, HK1, PSMD4, CLTC, MAP4K4, PROS1, DLD, SDHB, GNAS, COPS7A, MPC2, HEBP1, BLVRA, ID2, SCAND1, ETFB, MRPS16, PIN1, TRAK2, AMDHD2, PLEKHJ1, BZW2, PCNA, WDR61, RFC5, OXA1L, MCM3, CEP57, PSMF1, POLR2K, PSMD2, ATP6V1D, PSMD9, AKAP8L, GRN, SPAG7, ENOSF1, PCK2, PCCB, NOLC1, EBNA1BP2, CD58, RFC2, ASAH1, LAGE3, AKR7A2, and RSU1.

In some embodiments, altering the gene signature comprises decreased expression and/or decreased activity in the progenitor cell of one or more genes selected from the genes designated as a “down” gene in the gene directionality column of Table 1a. In some embodiments, the one or more genes selected from the genes designated as a “down” gene in the gene directionality column of Table 1a comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, or 26 or more genes selected from the genes designated as a “down” gene in the gene directionality column of Table 1a. In some embodiments, the one or more genes selected from the genes designated as a “down” gene in the gene directionality column of Table 1a comprises at least one of NUCB2, XBP1, CCNB1, CDC20, PLK1, CDK6, ITGB1BP1, CCNE2, PTPN6, CBR1, HLA-DRA, MAP7, SOX4, CASP3, DNAJB6, HOXA10, IL1B, ICAM3, ADGRG1, HLA-DMA, PDLIM1, PSMB8, EPB41L2, RPL39L, PYGL, CYB561, and HOMER2.

In some embodiments, altering the gene signature comprises increased expression and/or increased activity and/or decreased expression and/or decreased activity in the progenitor cell of one or more genes selected from Table 1b. In some embodiments, the one or more genes comprises 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or 41 or more, or 42 or more, or 43 or more, or 44 or more, or 45 or more, or 46 or more, or 47 or more, or 48 or more, or 49 or more, 50 or more, 51 or more, or 52 or more, or 53 or more, or 54 or more, or 55 or more, or 56 or more, or 57 or more, or 58 or more, or 59 or more, 60 or more, or 61 or more, or 62 or more, or 63 or more, or 64 or more, or 65 or more, or 66 or more, or 67 or more, or 68 or more, or 69 or more, 70 or more, or 71 or more, or 72 or more, or 73 or more, or 74 or more, or 75 or more, or 76 or more, or 77 or more, or 78 or more, or 79 or more, 80 or more, or 81 or more, or 82 or more, or 83 or more, or 84 or more, or 85 or more, or 86 or more, or 87 or more, or 88 or more, or 89 or more, 90 or more, or 91 or more, or 92 or more, or 93 or more, or 94 or more, or 95 or more, or 96 or more, or 97 or more, or 98 or more, or 99 or more, or 100 or more, or 101 or more, or 102 or more, or 103 or more, or 104 or more, or 105 or more, or 106 or more, or 107 or more, or 108 or more, or 109 or more, or 110 or more, or 111 or more, or 112 or more, or 113 or more, or 114 or more, or 115 or more, or 116 or more, or 117 or more, or 118 or more, or 119 or more, or 120 or more, or 121 or more, or 122 or more, or 123 or more, or 124 or more, or 125 or more, or 126 or more, or 127 or more, or 128 or more, or 129 or more, or 130 or more, or 131 or more, or 132 or more, 133 or more, or 134 or more, or 135 or more, or 136 or more, or 137 or more, or 138 or more, or 139 or more, or 140 or more, or 141 or more, or 142 or more, or 143 or more, or 144 or more, or 145 or more, or 146 or more, or 147 or more, or 148 or more, or 149 or more, 150 or more, 151 or more, or 152 or more, or 153 or more, or 154 or more, or 155 or more, or 156 or more, or 157 or more, or 158 or more, or 159 or more, 160 or more, or 161 or more, or 162 or more, or 163 or more, or 164 or more, or 165 or more, or 166 or more, or 167 or more, or 168 or more, or 169 or more, 170 or more, or 171 or more, or 172 or more, or 173 or more, or 174 or more, or 175 or more, or 176 or more, or 177 or more, or 178 or more, or 179 or more, 180 or more, or 181 or more, or 182 or more, or 183 or more, or 184 or more, or 185 or more, or 186 or more, or 187 or more, or 188 or more, or 189 or more, 190 or more, or 191 or more, or 192 or more, or 193 or more, or 194 or more, or 195 or more, or 196 or more, or 197 or more, or 198 or more, or 199 or more, or 200 or more, or 201 or more, or 202 or more, or 203 or more, or 204 or more, or 205 or more, or 206 or more, or 207 or more, or 208 or more, or 209 or more, or 210 or more, or 211 or more, or 212 or more, or 213 or more, or 214 or more, or 215 or more, or 216 or more, or 217 or more, or 218 or more, or 219 or more, or 220 or more, or 221 or more, or 222 or more, or 223 or more, or 224 or more, or 225 or more, or 226 or more, or 227 or more, or 228 or more, or 229 or more, or 230 or more, or 231 or more, or 232 or more, or 233 or more, or 234 or more, or 235 or more, or 236 or more, or 237 or more, or 238 or more, or 239 or more, or 240 or more, or 241 or more, or 242 or more, or 243 or more, or 244 or more, or 245 or more, or 246 or more, or 247 or more, or 248 or more, or 249 or more, 250 or more, 251 or more, or 252 or more, or 253 or more, or 254 or more, or 255 or more, or 256 or more, or 257 or more, or 258 or more, or 259 or more, 260 or more, or 261 or more, or 262 or more, or 263 or more, or 264 or more, or 265 or more, or 266 or more, or 267 or more, or 268 or more, or 269 or more, 270 or more, or 271 or more, or 272 or more genes selected from Table 1b. In some embodiments, the one or more genes comprises at least one of RAP1GAP, E2F2, RSRP1, RHD, RHCE, ERMAP, SLC2A1, CD58, SELENBP1, PPOX, NPL, ADIPOR1, BTG2, KLHDC8A, SDE2, GUK1, LBH, LTBP1, ZC3H6, TRAK2, STRADB, TMBIM1, DNAJB2, KAT2B, ABHD5, CPOX, RAB6B, PAQR9, SIAH2, NCEH1, KLF3, FRYL, MOB1B, HERC6, TSPAN5, GYPE, GYPB, FHDC1, CLCN3, ANKH, EPB41L4A, IRF1, CYSTM1, FAXDC2, TRIM10, TSPO2, CCND3, GTPBP2, GCLC, FOXO3, SERINC1, CITED2, JAZF1, MTURN, CD36, PNPLA8, BPGM, KDM7A, MFHAS1, CTSB, SLC25A37, BNIP3L, RNF19A, GRINA, HSF1, AQP3, CTSL, TMOD1, STOM, RXRA, OPTN, FRMD4A, STAM, MXI1, UROS, RIC8A, ILK, SOX6, CAT, YPEL4, UCP2, PPME1, ENDOD1, PTMS, CMAS, NFE2, KIF5A, RAB3IP, NUDT4, HECTD4, SDSL, RB1, PNP, DCAF11, ATP6V1D, DPF3, ZFYVE21, KIF26A, KLF13, CCNDBP1, EPB42, REXO5, ITFG1, TERF2IP, SLC7A5, EPN2,NATD1, PLEKHH3, SLC4A1, FAM117A, WIPI1, SMIM5, LPIN2, RIOK3, UBXN6, 2-Mar, JUNB, AKAP8L, UPK1A-AS1, PPP1R15A, GPCPD1, FAM210B, RBM38, TUBB1, ITSN1, GRAP2, WWC3, ALAS2, FAM122C, MOSPD1, SOX4, CLSTN1, PGM1, CD2, PHGDH, MLLT11, IFI16, FCER1A, RGS18, CD34, NENF, PLEK, IL1B, IGFBP7, INPP4B, BASP1, FYB1, CD74, SERPINB6, TUBB2B, LTB, LST1, AIF1, MPIG6B, HLA-DRA, HLA-DRB1, HLA-DMA, HLA-DPA1, HLA-DPB1, MAP7, CPVL, SCRN1, GNAI1, CYP3A4, PRKAG2, CLU, PVT1, ALDH1A1, NFIL3, DNM1, FAM69B, NPDC1, VIM, ARID5B, ZMIZ1, SESN2, GADD45A, DENND2D, FAM91A3P, S100A6, IER5, RGS16, PHLDA3, XPC, LXN, ZMAT3, CDKN1A, SESN1, PLIN2, RPS6, CDKN2A, ANKRD18A, LCN12, FAS, CTSD, HBBP1, DDB2, CTTN, SNORD15B, MDM2, PXMP2, PLEK2, RPS27L, LYRM1, RNF167, RNU4-34P, MYL4, FDXR, TNFSF9, CD70, GDF15, ECH1, BBC3, BAX, FTL, RPS5, ADA, GNAS, APOBEC3H, RHOC, CYP1B1, SUCNR1, TIPARP, HSD17B11, HLA-A, HLA-B, PSMB9, ASAH1, VPS28, HACD1, BGLT3, HBG1, HBG2, TRIM22, PRDX5, TSC22D1, RGS6, IFI27L2, B2M, ARID3A, RABAC1, and BEX1.

In some embodiments, altering the gene signature comprises increased expression and/or increased activity in the progenitor cell of one or more genes selected from the genes designated as an “up” gene in the gene directionality column of Table 2. In some embodiments, the one or more genes selected from the genes designated as an “up” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, or 19 or more genes. In another embodiment, the one or more genes selected from the genes designated as an “up” gene in the gene directionality column of Table 2 comprises at least one of HMGA1, KLF1, KLF6, SREBF1, NFE2, ARID3A, GFI1B, KLF13, MLXIP, E2F8, MYBL2, HSF1, GMEB1, NFX1, TGIF1, KLF3, SP1, CENPX, HES6, and LIN28B. In some embodiments, altering the gene signature further comprises increasing the expression of a transcription factor of the gene.

In some embodiments, altering the gene signature comprises decreased expression and/or decreased activity in the progenitor cell of one or more genes selected from the genes designated as a “down” gene in the gene directionality column of Table 2. In some embodiments, the one or more genes selected from the genes designated as a “down” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, or 19 or more genes selected from the genes designated as a “down” gene in the gene directionality column of Table 2. In some embodiments, the one or more genes selected from the genes designated as a “down” gene in the gene directionality column of Table 2 comprises at least one of HOXA10, XBP1, SOX4, ZNF385D, NFIC, BATF, HHEX, RARG, KDM5B, ZFX, SPI1, TEAD4, SATB1, NFIX, PLAGL1, MEF2C, ZBTB1, HOXA9, THAP5, and ZFP57. In some embodiments, altering the gene signature further comprises increasing the expression of a transcription factor of the gene.

In some embodiments, an increase in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about: 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, or more decrease in gene expression relative to a cell that has not been contacted with a perturbagen and/or relative to a cell that has been contacted with a no treatment control (including DMSO).

In various embodiments, an increase in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater increase in gene expression relative to a cell that has not been contacted with a perturbagen and/or relative to a cell that has been contacted with a no treatment control (including DMSO). Likewise, a decrease in gene expression (e.g., the amount of mRNA expressed) may be about: a 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, or greater decrease in gene expression relative to a cell that has not been contacted with a perturbagen and/or relative to a cell that has been contacted with a no treatment control (including DMSO).

In an aspect, the present disclosure provides a method for promoting the formation of a erythrocytes or an immediate progenitor thereof. The method includes a step of exposing a starting population of stem/progenitor cells comprising a non-lineage committed CD34⁺ cell to a perturbation having a perturbation signature that promotes the transition of the starting population of stem/progenitor cells into a HbF-expressing proerythroblasts, early erythroblasts, intermediate erythroblasts, and/or late erythroblasts. In some embodiments, the at least one perturbagen is selected from Table 3, or a variant thereof.

In an aspect, the present disclosure provides a method for promoting the formation of a erythrocytes or an immediate progenitor thereof. The method includes a step of exposing a starting population of stem/progenitor cells comprising a non-lineage committed CD34⁺ cell to a perturbation having a perturbation signature that promotes the transition of the starting population of stem/progenitor cells into a HbF-expressing proerythroblasts, early erythroblasts, intermediate erythroblasts, and/or late erythroblasts. In embodiments, the perturbation signature comprises increased expression and/or activity of one or more of genes selected from the genes designated as an “up” gene in the gene directionality column of Table 1a and/or Table 2 and/or a decreased expression and/or activity in the non-lineage committed CD34⁺ cell of one or more genes selected from the genes designated as a “down” gene in the gene directionality column of Table 1a and/or Table 2 and/or increased expression and/or activity of one or more of genes selected from Table 1b and/or a decreased expression and/or activity in the non-lineage committed CD34⁺ cell of one or more genes selected from Table 1b. Embodiments associated with the above aspects are likewise relevant to the present aspect. In other words, each of the embodiment mentioned above for the above aspects may be revised/adapted to be applicable to the present aspect.

In another aspect, the present disclosure provides a method of increasing a quantity of reticulocytes comprising HbF and/or erythrocytes or progenitors thereof. The method includes a step of exposing a starting population of stem/progenitor cells comprising a non-lineage committed CD34⁺ cell to a pharmaceutical composition that promotes the formation of lineage specific progenitor population selected from proerythroblasts, early erythroblasts, intermediate erythroblasts, and/or late erythroblasts. The pharmaceutical composition promotes the transition of a primitive stem/progenitor population into the lineage specific progenitor population that has the capacity to differentiate into reticulocytes comprising HbF and/or erythrocytes or progenitors thereof. In embodiments, the pharmaceutical composition comprises at least one perturbagen selected from Table 3, or a variant thereof. Embodiments associated with the above aspects are likewise relevant to the present aspect. In other words, each of the embodiment mentioned above for the above aspects may be revised/adapted to be applicable to the present aspect.

In yet another aspect, the present disclosure provides a perturbagen for use in any herein disclosed method.

In a further aspect, the present disclosure provides a pharmaceutical composition comprising perturbagen for use in any herein disclosed method.

### Methods and Perturbagens for Treating a Disease or Disorder

The major β-globin disorders, for example, sickle cell disease (SCD) and β-thalassemia, are classical Mendelian anemias caused by mutations in the adult β-globin gene. β-thalassemia results from mutations that decrease or ablate β-globin production. A single amino acid alteration, glutamine-valine substitution at codon 6 in the β-globin protein, leads to SCD and polymerization of deoxy sickle hemoglobin (HbS). Patients with elevated levels of fetal hemoglobin (also known as hemoglobin F, HbF, γ-globin) as adults exhibit reduced symptoms and enhanced survival.

HbF is the major hemoglobin produced during fetal life but is largely replaced by adult hemoglobin (HbA) following a “switch” around birth in normal individuals. In individuals having SCD, HbF is replaced by HbS. In most adults, HbF production is restricted to a small number of erythroid precursors and their progeny in the blood are F-cells. The γ-globin genes of fetal hemoglobin can be reactivated in adult individuals by several pharmacologic means and physiological manipulations.

Hydroxyurea (HU), a FDA-approved drug for SCD, is a potent inducer for HbF (γ-globin gene expression). While HU is effective for many patients, its utility is limited due to unpredictable induction of HbF and marginal benefit for patients with β-thalassemia. There exists a need for more effective therapies based on HbF reactivation. It is believed that HU induces the expression of HbF via the mechanism of action as a potent inhibitor of ribonucleotide reductase.

In some embodiments, the perturbagen of the present disclosure synergizes with HU. In some embodiments, the perturbagen of the present disclosure have substantially similar biological activity (e.g., induces a similar: cell behavior, gene expression profile, physiological response, or a combination of 1, 2, or all 3 of the foregoing) as HU. In some embodiments, one or more glucocorticoid receptor agonists synergize with HU or a perturbagen of the present disclosure. In some embodiments, one or more glucocorticoid receptor agonists have substantially similar biological activity as HU. In some embodiments, one or more glucocorticoids synergize with HU or a perturbagen of the present disclosure. In some embodiments, one or more glucocorticoids have substantially similar biological activity as HU. In some embodiments, the perturbagen is one or more glucocorticoid receptor agonists. In some embodiments, the perturbagen is used in combination with one or more glucocorticoid receptor agonists. In some embodiments, the glucocorticoid receptor agonist is selected from mapracorat and desoximetasone. In some embodiments, the perturbagen is used in combination with one or more glucocorticoids. In some embodiments, the glucocorticoid is dexamethasone.

In some embodiments, the therapeutic induction of HbF is through the transcriptional regulation of the human globin genes, for example, regulating chromatin modifying enzymes such as histone deacetylase (HDACs) with epigenetic regulators including selective and/or non-selective HDAC inhibitors (Brander et al., Chemical genetic strategy identifies histone deacetylase 1 (HDAC1) and HDAC 2 as therapeutic targets in sickle cell disease, PNAS, 2010, vol. 107, pp. 12617-12622). In some embodiments, the therapeutic induction of HbF is through the inhibition of BCL11A and KLF1 gene expressions (Steinberg et al., Blood, 2014, vol. 123, pp. 481-485).

The complexity of the HbF regulation suggests that combination therapy of different agents (e.g. one or more perturbagens, and/or another agent described herein, each optionally with a different mechanism of action, is an effective strategy for the induction of very high level of HbF while optionally limiting adverse effects. For example, combined therapy with hydroxyurea and recombinant erythropoietin elevates HbF level more than hydroxyurea alone in SCD patients (Roger et al., N Engl. J. Med. 1993, vol. 328, pp. 73-80).

In some embodiments, the present methods of treatment further comprise administration of one or more of recombinant erythropoietin and hydroxyurea. In some embodiments, the present methods of treatment involve a subject undergoing treatment with one or more of recombinant erythropoietin and hydroxyurea.

In some embodiments, the present methods of treatment further comprise administration of a P-selectin antibody, e.g. crizanlizumab. In some embodiments, the present methods of treatment involve a subject undergoing treatment with a P-selectin antibody, e.g. crizanlizumab.

An aspect of the present disclosure is a method for treating a disease or disorder characterized by an abnormal oxygen delivery. In this aspect, the method comprises the step of administering to a subject in need thereof a therapeutically effective amount of at least one perturbagen capable of altering a gene associated with at least one functionality in a progenitor cell.

Another aspect of the present disclosure is a method for treating a disease or disorder characterized by an abnormal oxygen delivery. In this aspect, the method comprises the step of administering to a subject in need thereof a cell, the cell having been contacted with at least one perturbagen capable of altering a gene associated with at least one functionality in a progenitor cell.

An aspect of the present disclosure is a method for treating a disease or disorder characterized by an abnormal oxygen delivery. In this aspect, the method comprises the step of administering to a subject in need thereof a therapeutically effective amount of a combination therapy having hydroxyurea and at least one perturbagen, wherein the combination therapy is capable of altering a gene associated with at least one functionality in a progenitor cell.

An aspect of the present disclosure is a method for treating a disease or disorder characterized by a hemoglobin deficiency. In this aspect, the method comprises the step of administering to a subject in need thereof a therapeutically effective amount of at least one perturbagen capable of altering a gene associated with at least one functionality in a progenitor cell.

Another aspect of the present disclosure is a method for treating a disease or disorder characterized by a hemoglobin deficiency. In this aspect, the method comprises the step of administering to a subject in need thereof a cell, the cell having been contacted with at least one perturbagen capable of altering a gene associated with at least one functionality in a progenitor cell.

An aspect of the present disclosure is a method for treating a disease or disorder characterized by a hemoglobin deficiency. In this aspect, the method comprises the step of administering to a subject in need thereof a therapeutically effective amount of a combination therapy having hydroxyurea and at least one perturbagen, wherein the combination therapy is capable of altering a gene associated with at least one functionality in a progenitor cell. Another aspect of the present disclosure is a method for treating a disease or disorder characterized by a hemoglobin deficiency. In this aspect, the method comprises the step of administering to a subject in need thereof a cell, the cell having been contacted with a combination therapy having hydroxyurea and at least one perturbagen, wherein the combination therapy is capable of altering a gene associated with at least one functionality in a progenitor cell.

An aspect of the present disclosure is a method for treating or preventing a sickle cell disease or a thalassemia. In this aspect, the method comprises the step of administering to a subject in need thereof a therapeutically effective amount of at least one perturbagen capable of altering a gene associated with at least one functionality in a progenitor cell.

Another aspect of the present disclosure is a method for treating or preventing a sickle cell disease or a thalassemia. In this aspect, the method comprises the step of administering to a subject in need thereof a cell, the cell having been contacted with at least one perturbagen capable of altering a gene associated with at least one functionality in a progenitor cell.

An aspect of the present disclosure is a method for treating or preventing a sickle cell disease or a thalassemia. In this aspect, the method comprises the step of administering to a subject in need thereof a therapeutically effective amount of a combination therapy of hydroxyurea and at least one perturbagen, wherein the combination therapy is capable of altering a gene associated with at least one functionality in a progenitor cell. Another aspect of the present disclosure is a method for treating or preventing a sickle cell disease or a thalassemia. In this aspect, the method comprises the step of administering to a subject in need thereof a cell, the cell having been contacted with a combination therapy of hydroxyurea and at least one perturbagen, wherein the combination therapy is capable of altering a gene associated with at least one functionality in a progenitor cell.

An aspect of the present disclosure is a method for treating a disease or disorder characterized by an abnormal oxygen delivery. The method comprises the steps of: (a) administering to a subject in need thereof a therapeutically effective amount of at least one perturbagen selected from Table 3, or a variant thereof, wherein the at least one perturbagen is capable of altering a gene associated with at least one functionality in a progenitor cell or (b) administering to a subject in need thereof a cell, the cell having been contacted with at least one perturbagen selected from Table 3, or a variant thereof.

In another aspect, the present disclosure provides a method for treating a disease or disorder characterized by a hemoglobin deficiency. The method comprises the steps of: (a) administering to a subject in need thereof a therapeutically effective amount of at least one perturbagen selected from Table 3, or a variant thereof, wherein the at least one perturbagen is capable of altering a gene associated with at least one functionality in a progenitor cell or (b) administering to a subject in need thereof a cell, the cell having been contacted with at least one perturbagen selected from Table 3, or a variant thereof. In some embodiments, the hemoglobin deficiency is an abnormal and/or reduced oxygen delivery functionality of hemoglobin, optionally resultant from mutations in one or more beta-like hemoglobin subunit genes, an exemplary mutation being in the adult HBB gene.

An aspect of the present disclosure is a method for treating a disease or disorder characterized by an abnormal oxygen delivery. In this aspect, the method comprises the step of administering to a subject in need thereof a therapeutically effective amount of at least one perturbagen of Table 3, or a variant thereof.

Another aspect of the present disclosure is a method for treating a disease or disorder characterized by an abnormal oxygen delivery. In this aspect, the method comprises the step of administering to a subject in need thereof a cell, the cell having been contacted with at least one perturbagen of Table 3.

An aspect of the present disclosure is a method for treating a disease or disorder characterized by an abnormal oxygen delivery. In this aspect, the method comprises the step of administering to a subject in need thereof a therapeutically effective amount of a combination therapy having hydroxyurea and at least one perturbagen selected from Table 3.

Another aspect of the present disclosure is a method for treating a disease or disorder characterized by an abnormal oxygen delivery. In this aspect, the method comprises the step of administering to a subject in need thereof a cell, the cell having been contacted with a combination therapy having hydroxyurea and at least one perturbagen selected from Table 3.

An aspect of the present disclosure is a method for treating a disease or disorder characterized by a hemoglobin deficiency. In this aspect, the method comprises the step of administering to a subject in need thereof a therapeutically effective amount of at least one perturbagen selected from Table 3.

Another aspect of the present disclosure is a method for treating a disease or disorder characterized by a hemoglobin deficiency. In this aspect, the method comprises the step of administering to a subject in need thereof a cell, the cell having been contacted with at least one perturbagen selected from Table 3.

An aspect of the present disclosure is a method for treating a disease or disorder characterized by a hemoglobin deficiency. In this aspect, the method comprises the step of administering to a subject in need thereof a therapeutically effective amount of a combination therapy having hydroxyurea and at least one perturbagen selected from Table 3. Another aspect of the present disclosure is a method for treating a disease or disorder characterized by a hemoglobin deficiency. In this aspect, the method comprises the step of administering to a subject in need thereof a cell, the cell having been contacted with a combination therapy having hydroxyurea and at least one perturbagen selected from Table 3.

An aspect of the present disclosure is a method for treating or preventing a sickle cell disease or a thalassemia. In this aspect, the method comprises the step of administering to a subject in need thereof a therapeutically effective amount of at least one perturbagen selected from Table 3.

Another aspect of the present disclosure is a method for treating or preventing a sickle cell disease or a thalassemia. In this aspect, the method comprises the step of administering to a subject in need thereof a cell, the cell having been contacted with at least one perturbagen selected from Table 3.

An aspect of the present disclosure is a method for treating or preventing a sickle cell disease or a thalassemia. In this aspect, the method comprises the step of administering to a subject in need thereof a therapeutically effective amount of a combination therapy of hydroxyurea and at least one perturbagen selected from Table 3. Another aspect of the present disclosure is a method for treating or preventing a sickle cell disease or a thalassemia. In this aspect, the method comprises the step of administering to a subject in need thereof a cell, the cell having been contacted with a combination therapy of hydroxyurea and at least one perturbagen selected from Table 3.

An aspect of the present disclosure is a method for treating a disease or disorder characterized by an abnormal oxygen delivery. In embodiments, the method comprises the steps of: (a) administering to a subject in need thereof a therapeutically effective amount of at least one perturbagen selected from Table 3, or a variant thereof, or (b) administering to a subject in need thereof a cell, the cell having been contacted with at least one perturbagen selected from Table 3, or a variant thereof.

In another aspect, the present disclosure provides a method for treating a disease or disorder characterized by a hemoglobin deficiency. In embodiments, the method comprises the steps of: (a) administering to a subject in need thereof a therapeutically effective amount of at least one perturbagen selected from Table 3, or a variant thereof, or (b) administering to a subject in need thereof a cell, the cell having been contacted with at least one perturbagen selected from Table 3, or a variant thereof. In some embodiments, the hemoglobin deficiency is an abnormal and/or reduced oxygen delivery functionality of hemoglobin, optionally resultant from mutations in one or more beta-like hemoglobin subunit genes, an exemplary mutation being in the adult HBB gene.

In some embodiments, for any herein disclosed method, the disease or disorder characterized by an abnormal oxygen delivery and/or a hemoglobin deficiency is an anemia.

In some embodiments, for any herein disclosed method, the administering is directed to the bone marrow of the subject. In some embodiments, for any herein disclosed method, the administering is via intraosseous injection or intraosseous infusion. In some embodiments, for any herein disclosed method, the administering the cell is via intravenous injection or intravenous infusion. In some embodiments, the administering is simultaneously or sequentially to one or more mobilization agents.

In another aspect, the present disclosure provides a method for treating or preventing a sickle cell disease or a thalassemia. The method comprises the steps of: (a) administering to a subject in need thereof a therapeutically effective amount of at least one perturbagen selected from Table 3, or a variant thereof, where the at least one perturbagen is capable of altering a gene associated with at least one functionality in a progenitor cell or (b) administering to a subject in need thereof a cell, the cell having been contacted with at least one perturbagen selected from Table 3, or a variant thereof.

An aspect of the present disclosure is a method for treating a sickle cell disease or a thalassemia. In this aspect, the method comprises the steps of administering to a subject in need thereof a therapeutically effective amount of at least one perturbagen capable of altering a gene associated with at least one functionality in a progenitor cell.

Another aspect of the present disclosure is a method for treating a sickle cell disease or a thalassemia. In this aspect, the method comprises the steps of administering to a subject in need thereof a cell, the cell having been contacted with at least one perturbagen capable of altering a gene associated with at least one functionality in a progenitor cell.

An aspect of the present disclosure is a method for treating a sickle cell disease or a thalassemia. In this aspect, the method comprises the steps of administering to a subject in need thereof a therapeutically effective amount of a combination therapy of hydroxyurea and at least one perturbagen, wherein the combination therapy is capable of altering a gene associated with at least one functionality in a progenitor cell. Another aspect of the present disclosure is a method for treating a sickle cell disease or a thalassemia. In this aspect, the method comprises the steps of administering to a subject in need thereof a cell, the cell having been contacted with a combination therapy of hydroxyurea and at least one perturbagen, wherein the combination therapy is capable of altering a gene associated with at least one functionality in a progenitor cell.

An aspect of the present disclosure is a method for treating a disease or disorder characterized by an abnormal oxygen delivery. In this aspect, the method comprises the steps of administering to a subject in need thereof a therapeutically effective amount of at least one perturbagen selected from Table 3, or a variant thereof, wherein the perturbagen is capable of altering a gene associated with at least one functionality in a progenitor cell and inducing the (CD34+/CD41^{Low}+/CD235a+) cell state in the progenitor cell. Another aspect of the present disclosure is a method for treating a disease or disorder characterized by an abnormal oxygen delivery. In this aspect, the method comprises the steps of administering to a subject in need thereof a cell, the cell having been contacted with a therapeutically effective amount of at least one perturbagen selected from Table 3, or a variant thereof, wherein the perturbagen is capable of altering a gene associated with at least one functionality in a progenitor cell and inducing the (CD34+/CD41^{Low}+/CD235a+) cell state in the cell.

An aspect of the present disclosure is a method for treating a disease or disorder characterized by an abnormal oxygen delivery. In this aspect, the method comprises the steps of administering to a subject in need thereof a combination of a therapeutically effective amount of hydroxyurea (HU) and a therapeutically effective amount of at least one perturbagen selected from Table 3, or a variant thereof, wherein the perturbagen is capable of altering a gene associated with at least one functionality in a progenitor cell and inducing the (CD34+/CD41^{Low}+/CD235a+) cell state in the progenitor cell. Another aspect of the present disclosure is a method for treating a disease or disorder characterized by an abnormal oxygen delivery. In this aspect, the method comprises the steps of administering to a subject in need thereof a cell, the cell having been contacted with a combination of a therapeutically effective amount of hydroxyurea (HU) and a therapeutically effective amount of at least one perturbagen selected from Table 3, or a variant thereof, wherein the perturbagen is capable of altering a gene associated with at least one functionality in a progenitor cell and inducing the (CD34+/CD41^{Low}+/CD235a+) cell state in the cell.

In another aspect, the present disclosure provides a method for treating or preventing a sickle cell disease or a thalassemia. In embodiments, the method comprises the steps of: (a) administering to a subject in need thereof a therapeutically effective amount of at least one perturbagen selected from Table 3, or a variant thereof, or (b) administering to a subject in need thereof a cell, the cell having been contacted with at least one perturbagen selected from Table 3, or a variant thereof.

An aspect of the present disclosure is a method for treating a disease or disorder characterized by an abnormal oxygen delivery. In an aspect, the method comprises the steps of administering to a subject in need thereof a therapeutically effective amount of at least one perturbagen selected from Table 3, or a variant thereof, wherein the perturbagen is capable of inducing the (CD34+/CD41^{Low}+/CD235a+) cell state in the progenitor cell. Another aspect of the present disclosure is a method for treating a disease or disorder characterized by an abnormal oxygen delivery. In an aspect, the method comprises the steps of administering to a subject in need thereof a cell, the cell having been contacted with a therapeutically effective amount of at least one perturbagen selected from Table 3, or a variant thereof, wherein the perturbagen is capable of inducing the (CD34+/CD41^{Low}+/CD235a+) cell state in the cell.

An aspect of the present disclosure is a method for treating a disease or disorder characterized by an abnormal oxygen delivery. In an aspect, the method comprises the steps of administering to a subject in need thereof a combination of a therapeutically effective amount of hydroxyurea (HU) and a therapeutically effective amount of at least one perturbagen selected from Table 3, or a variant thereof, wherein the perturbagen is capable of inducing the (CD34+/CD41^{Low}+/CD235a+) cell state in the cell. Another aspect of the present disclosure is a method for treating a disease or disorder characterized by an abnormal oxygen delivery. In an aspect, the method comprises the steps of administering to a subject in need thereof a cell, the cell having been contacted with a combination of a therapeutically effective amount of hydroxyurea (HU) and a therapeutically effective amount of at least one perturbagen selected from Table 3, or a variant thereof, wherein the perturbagen is capable of inducing the (CD34+/CD41^{Low}+/CD235a+) cell state in the cell.

An aspect of the present disclosure is a method for treating a disease or disorder characterized by an abnormal oxygen delivery. In this aspect, the method comprises the steps of administering to a subject in need thereof a combination of a therapeutically effective amount of hydroxyurea (HU) and a therapeutically effective amount of one or more glucocorticoids and/or one or more glucocorticoid receptor agonists and inducing the (CD34+/CD41^{Low}+/CD235a+) cell state in the progenitor cell. Another aspect of the present disclosure is a method for treating a disease or disorder characterized by an abnormal oxygen delivery. In this aspect, the method comprises the steps of administering to a subject in need thereof a cell, the cell having been contacted with a combination of a therapeutically effective amount of hydroxyurea (HU) and a therapeutically effective amount of one or more glucocorticoids and/or one or more glucocorticoid receptor agonists and inducing the (CD34+/CD41^{Low}+/CD235a+) cell state in the cell. In some embodiments, the glucocorticoid is dexamethasone. In some embodiments, the glucocorticoid receptor agonist is selected from mapracorat and desoximetasone.

An aspect of the present disclosure is a method for treating a disease or disorder characterized by a hemoglobin deficiency. In this aspect, the method comprises the steps of administering to a subject in need thereof a therapeutically effective amount of at least one perturbagen selected from Table 3, or a variant thereof, wherein the perturbagen is capable of altering a gene associated with at least one functionality in a progenitor cell and inducing the (CD34+/CD41^{Low}+/CD235a+) cell state in the progenitor cell. Another aspect of the present disclosure is a method for treating a disease or disorder characterized by a hemoglobin deficiency. In this aspect, the method comprises the steps of administering to a subject in need thereof a cell, the cell having been contacted with a therapeutically effective amount of at least one perturbagen selected from Table 3, or a variant thereof, wherein the perturbagen is capable of altering a gene associated with at least one functionality in a progenitor cell and inducing the (CD34+/CD41^{Low}+/CD235a+) cell state in the cell.

An aspect of the present disclosure is a method for treating a disease or disorder characterized by a hemoglobin deficiency. In this aspect, the method comprises the steps of administering to a subject in need thereof a combination of a therapeutically effective amount of hydroxyurea (HU) and a therapeutically effective amount of at least one perturbagen selected from Table 3, or a variant thereof, wherein the perturbagen is capable of altering a gene associated with at least one functionality in a progenitor cell and inducing the (CD34+/CD41^{Low}+/CD235a+) cell state in the progenitor cell. Another aspect of the present disclosure is a method for treating a disease or disorder characterized by a hemoglobin deficiency. In this aspect, the method comprises the steps of administering to a subject in need thereof a cell, the cell having been contacted with a combination of a therapeutically effective amount of hydroxyurea (HU) and a therapeutically effective amount of at least one perturbagen selected from Table 3, or a variant thereof, wherein the perturbagen is capable of altering a gene associated with at least one functionality in a progenitor cell and inducing the (CD34+/CD41^{Low}+/CD235a+) cell state in the cell.

An aspect of the present disclosure is a method for treating a disease or disorder characterized by a hemoglobin deficiency. In this aspect, the method comprises the steps of administering to a subject in need thereof a therapeutically effective amount of at least one perturbagen selected from Table 3, or a variant thereof, wherein the perturbagen is capable of inducing the (CD34+/CD41^{Low}+/CD235a+) cell state in the cell. Another aspect of the present disclosure is a method for treating a disease or disorder characterized by a hemoglobin deficiency. In this aspect, the method comprises the steps of administering to a subject in need thereof a cell, the cell having been contacted with a therapeutically effective amount of at least one perturbagen selected from Table 3, or a variant thereof, wherein the perturbagen is capable of inducing the (CD34+/CD41^{Low}+/CD235a+) cell state in the cell.

An aspect of the present disclosure is a method for treating a disease or disorder characterized by a hemoglobin deficiency. In this aspect, the method comprises the steps of administering to a subject in need thereof a combination of a therapeutically effective amount of hydroxyurea (HU) and a therapeutically effective amount of at least one perturbagen selected from Table 3, or a variant thereof, wherein the perturbagen is capable of inducing the (CD34+/CD41^{Low}+/CD235a+) cell state in the cell. Another aspect of the present disclosure is a method for treating a disease or disorder characterized by a hemoglobin deficiency. In this aspect, the method comprises the steps of administering to a subject in need thereof a cell, the cell having been contacted with a combination of a therapeutically effective amount of hydroxyurea (HU) and a therapeutically effective amount of at least one perturbagen selected from Table 3, or a variant thereof, wherein the perturbagen is capable of inducing the (CD34+/CD41^{Low}+/CD235a+) cell state in the cell.

An aspect of the present disclosure is a method for treating a disease or disorder characterized by a hemoglobin deficiency. In this aspect, the method comprises the steps of administering to a subject in need thereof a combination of a therapeutically effective amount of hydroxyurea (HU) and a therapeutically effective amount of one or more glucocorticoids and/or one or more glucocorticoid receptor agonists and inducing the (CD34+/CD41^{Low}+/CD235a+) cell state in the progenitor cell. Another aspect of the present disclosure is a method for treating a disease or disorder characterized by a hemoglobin deficiency. In this aspect, the method comprises the steps of administering to a subject in need thereof a cell, the cell having been contacted with a combination of a therapeutically effective amount of hydroxyurea (HU) and a therapeutically effective amount of one or more glucocorticoids and/or one or more glucocorticoid receptor agonists and inducing the (CD34+/CD41^{Low}+/CD235a+) cell state in the cell. In some embodiments, the glucocorticoid is dexamethasone. In some embodiments, the glucocorticoid receptor agonist is selected from mapracorat and desoximetasone.

An aspect of the present disclosure is a method for treating or preventing a sickle cell disease or a thalassemia. In this aspect, the method comprises the steps of administering to a subject in need thereof a therapeutically effective amount of at least one perturbagen selected from Table 3, or a variant thereof, wherein the perturbagen is capable of altering a gene associated with at least one functionality in a progenitor cell and inducing the (CD34+/CD41^{Low}+/CD235a+) cell state in the progenitor cell. Another aspect of the present disclosure is a method for treating or preventing a sickle cell disease or a thalassemia. In this aspect, the method comprises the steps of administering to a subject in need thereof a cell, the cell having been contacted with a therapeutically effective amount of at least one perturbagen selected from Table 3, or a variant thereof, wherein the perturbagen is capable of altering a gene associated with at least one functionality in a progenitor cell and inducing the (CD34+/CD41^{Low}+/CD235a+) cell state in the cell.

An aspect of the present disclosure is a method for treating or preventing a sickle cell disease or a thalassemia. In this aspect, the method comprises the steps of administering to a subject in need thereof a combination of a therapeutically effective amount of hydroxyurea (HU) and a therapeutically effective amount of at least one perturbagen selected from Table 3, or a variant thereof, wherein the perturbagen is capable of altering a gene associated with at least one functionality in a progenitor cell and inducing the (CD34+/CD41^{Low}+/CD235a+) cell state in the progenitor cell. Another aspect of the present disclosure is a method for treating or preventing a sickle cell disease or a thalassemia. In this aspect, the method comprises the steps of administering to a subject in need thereof a cell, the cell having been contacted with a combination of a therapeutically effective amount of hydroxyurea (HU) and a therapeutically effective amount of at least one perturbagen selected from Table 3, or a variant thereof, wherein the perturbagen is capable of altering a gene associated with at least one functionality in a progenitor cell and inducing the (CD34+/CD41^{Low}+/CD235a+) cell state in the cell.

An aspect of the present disclosure is a method for treating or preventing a sickle cell disease or a thalassemia. In this aspect, the method comprises the steps of administering to a subject in need thereof a therapeutically effective amount of at least one perturbagen selected from Table 3, or a variant thereof, wherein the perturbagen is capable of inducing the (CD34+/CD41^{Low}+/CD235a+) cell state in the cell. Another aspect of the present disclosure is a method for treating or preventing a sickle cell disease or a thalassemia. In this aspect, the method comprises the steps of administering to a subject in need thereof a cell, the cell having been contacted with a therapeutically effective amount of at least one perturbagen selected from Table 3, or a variant thereof, wherein the perturbagen is capable of inducing the (CD34+/CD41^{Low}+/CD235a+) cell state in the cell.

An aspect of the present disclosure is a method for treating or preventing a sickle cell disease or a thalassemia. In this aspect, the method comprises the steps of administering to a subject in need thereof a combination of a therapeutically effective amount of hydroxyurea (HU) and a therapeutically effective amount of at least one perturbagen selected from Table 3, or a variant thereof, wherein the perturbagen is capable of inducing the (CD34+/CD41^{Low}+/CD235a+) cell state in the cell. Another aspect of the present disclosure is a method for treating or preventing a sickle cell disease or a thalassemia. In this aspect, the method comprises the steps of administering to a subject in need thereof a cell, the cell having been contacted with a combination of a therapeutically effective amount of hydroxyurea (HU) and a therapeutically effective amount of at least one perturbagen selected from Table 3, or a variant thereof, wherein the perturbagen is capable of inducing the (CD34+/CD41^{Low}+/CD235a+) cell state in the cell.

An aspect of the present disclosure is a method for treating or preventing a sickle cell disease or a thalassemia. In this aspect, the method comprises the steps of administering to a subject in need thereof a combination of a therapeutically effective amount of hydroxyurea (HU) and a therapeutically effective amount of one or more glucocorticoids and/or one or more glucocorticoid receptor agonists and inducing the (CD34+/CD41^{Low}+/CD235a+) cell state in the progenitor cell. Another aspect of the present disclosure is a method for treating or preventing a sickle cell disease or a thalassemia. In this aspect, the method comprises the steps of administering to a subject in need thereof a cell, the cell having been contacted with a combination of a therapeutically effective amount of hydroxyurea (HU) and a therapeutically effective amount of one or more glucocorticoids and/or one or more glucocorticoid receptor agonists and inducing the (CD34+/CD41^{Low}+/CD235a+) cell state in the cell. In some embodiments, the glucocorticoid is dexamethasone. In some embodiments, the glucocorticoid receptor agonist is selected from mapracorat and desoximetasone.

In some embodiments, for any herein disclosed method, the sickle cell disease or a thalassemia is beta-thalassemia (transfusion dependent). In some embodiments, for any herein disclosed method, the sickle cell disease or a thalassemia is beta-thalassemia major. In some embodiments, for any herein disclosed method, the sickle cell disease or a thalassemia is beta-thalassemia intermedia. In some embodiments, for any herein disclosed method, the sickle cell disease or a thalassemia is beta-thalassemia minor. In a further embodiment, for any herein disclosed method, the sickle cell disease or a thalassemia is sickle cell anemia (SS), sickle hemoglobin-C disease (SC), or sickle beta-plus thalassemia and sickle beta-zero thalassemia.

In some embodiments, for any herein disclosed method, the at least one perturbagen is capable of altering a gene associated with at least one functionality selected from the functionality of the genes designated as an “up” gene in the gene directionality column of Table 1a and/or the genes designated as a “down” gene in the gene directionality column of Table 1a.

In some embodiments, for any herein disclosed method, the at least one perturbagen is capable of altering a gene associated with at least one functionality selected from the functionality of the genes of Table 1b.

In some embodiments, for any herein disclosed method, the at least one perturbagen is capable of altering a gene associated with at least one functionality selected from the functionality of the genes designated as an “up” gene in the gene directionality column of Table 2 and/or the genes designated as a “down” gene in the gene directionality column of Table 2.

In an embodiment, the subject is selected by steps including: obtaining from the subject having the disease or disorder a sample of cells comprising a non-lineage committed CD34⁺ cell; and contacting the sample of cells with least one perturbagen selected from Table 3, or a variant thereof. In this aspect, the at least one perturbagen alters a gene associated with at least one functionality in the sample of cells. In some embodiments, the subject is selected by steps including: obtaining from a subject having the disease or disorder a sample of cells comprising a non-lineage committed CD34⁺ cell; and contacting the sample of cells with at least one perturbagen capable of altering a gene associated with at least one functionality in a non-lineage committed CD34⁺ cell. In this aspect, the at least one perturbagen increases in the sample of cells the expression and/or activity of a gene associated with at least one functionality selected from the functionality of the genes designated as an “up” gene in the gene directionality column of Table 1a and/or Table 2 and/or decreases in the sample of cells the expression and/or activity of one or more genes associated with at least one functionality selected from the functionality of the genes designated as a “down” gene in the gene directionality column of Table 1a and/or Table 2 and/or increases in the sample of cells the expression and/or activity of one or more genes selected from Table 1b and/or decreases in the sample of cells the expression and/or activity of one or more genes selected from Table 1b. In some embodiments, altering the gene signature further comprises increasing the expression of one or more transcription factors. In some embodiments, the expression and/or activity of a gene is increased and/or decreased by increasing the expression of one or more transcription factors.

In an embodiment, the subject is selected by steps including: obtaining from the subject having the disease or disorder a sample of cells comprising a non-lineage committed CD34⁺ cell; and contacting the sample of cells with least one perturbagen selected from Table 3, or a variant thereof. In this aspect, the at least one perturbagen increases in the sample of cells the expression and/or activity of a gene associated with at least one functionality selected from the functionality of the genes designated as an “up” gene in the gene directionality column of Table 1a and/or Table 2 and/or decreases in the sample of cells the expression and/or activity of one or more genes associated with at least one functionality selected from the functionality of the genes designated as a “down” gene in the gene directionality column of Table 1a and/or Table 2 and/or increases in the sample of cells the expression and/or activity of one or more genes selected from Table 1b and/or decreases in the sample of cells the expression and/or activity of one or more genes selected from Table 1b. In some embodiments, the expression and/or activity of a gene is increased and/or decreased by increasing the expression of one or more transcription factors.

In an embodiment, the subject is selected by steps including: obtaining from the subject having the disease or disorder a sample of cells comprising a non-lineage committed CD34⁺ cell; and contacting the sample of cells with least one perturbagen selected from Table 3, or a variant thereof. In this aspect, when the at least one perturbagen alters a gene associated with at least one functionality in the sample of cells, the subject is selected as a subject.

In some embodiments, the present disclosure provides a method for selecting the subject for the treatment described above, the method including the steps of: obtaining from a subject having the disease or disorder a sample of cells comprising a non-lineage committed CD34⁺ cell; and contacting the sample of cells with at least one perturbagen capable of altering a gene associated with at least one functionality in a non-lineage committed CD34⁺ cell. In this aspect, when the at least one perturbagen causes the increases in the sample of cells the expression and/or activity of a gene associated with at least one functionality selected from the functionality of the genes designated as an “up” gene in the gene directionality column of Table 1a and/or Table 2 and/or decreases in the sample of cells the expression and/or activity of one or more genes associated with at least one functionality selected from the functionality of the genes designated as a “down” gene in the gene directionality column of Table 1a and/or Table 2 and/or increases in the sample of cells the expression and/or activity of one or more genes selected from Table 1b and/or decreases in the sample of cells the expression and/or activity of one or more genes selected from Table 1b, the subject is selected as a subject. In some embodiments, the expression and/or activity of a gene is increased and/or decreased by increasing the expression of one or more transcription factors.

In an embodiment, the subject is selected by steps including: obtaining from the subject having the disease or disorder a sample of cells comprising a non-lineage committed CD34⁺ cell; and contacting the sample of cells with least one perturbagen selected from Table 3, or a variant thereof. In this aspect, when the at least one perturbagen causes the increases in the sample of cells the expression and/or activity of a gene associated with at least one functionality selected from the functionality of the genes designated as an “up” gene in the gene directionality column of Table 1a and/or Table 2 and/or decreases in the sample of cells the expression and/or activity of one or more genes associated with at least one functionality selected from the functionality of the genes designated as a “down” gene in the gene directionality column of Table 1a and/or Table 2 and/or increases in the sample of cells the expression and/or activity of one or more genes selected from Table 1b and/or decreases in the sample of cells the expression and/or activity of one or more genes selected from Table 1b, the subject is selected as a subject. In some embodiments, the expression and/or activity of a gene is increased and/or decreased by increasing the expression of one or more transcription factors. In some embodiments, the present disclosure provides a method for selecting the subject for the treatment described above, the method including the steps of: obtaining from a subject having the disease or disorder a sample of cells comprising a non-lineage committed CD34⁺ cell; and contacting the sample of cells with at least one perturbagen selected from Table 3, or a variant thereof. In this aspect, when the at least one perturbagen increases in the sample of cells the expression and/or activity of one or more genes selected from the genes designated as an “up” gene in the gene directionality column of Table 1a and/or Table 2 and/or decreases in the sample of cells the expression and/or activity of one or more genes selected from the genes designated as a “down” gene in the gene directionality column of Table 1a and/or Table 2 and/or increases in the sample of cells the expression and/or activity of one or more genes selected from Table 1b and/or decreases in the sample of cells the expression and/or activity of one or more genes selected from Table 1b, the subject is selected as a subject. In some embodiments, the expression and/or activity of a gene is increased and/or decreased by increasing the expression of one or more transcription factors. An aspect of the present disclosure provides use of the perturbagen of Table 3, or a variant thereof in the manufacture of a medicament for treating a disease or disorder characterized by an abnormal oxygen delivery or a hemoglobin deficiency. In another aspect, the present disclosure provides use of the perturbagen of Table 3, or a variant thereof in the manufacture of a medicament for treating sickle cell disease or a thalassemia.

An aspect of the present disclosure is related to a method of identifying a candidate perturbation for promoting the transition of a starting population of progenitor cells into erythrocytes comprising HbF or HbF-expressing progenitors thereof. The method include the steps of: exposing the starting population of progenitor cells to a perturbation identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into erythrocytes comprising HbF or HbF-expressing progenitors thereof following exposure of the population of cells to the perturbation, identifying the perturbation as a candidate perturbation for promoting the transition of a population of progenitor cells into erythrocytes comprising HbF or HbF-expressing progenitors thereof based on the perturbation signature. In this aspect, the perturbation signature is an increase in expression and/or activity in the progenitor cell of one or more genes selected from the genes designated as an “up” gene in the gene directionality column of Table 1a and/or Table 2, and/or a decrease in expression and/or activity in the progenitor cell of one or more genes selected from the genes designated as a “down” gene in the gene directionality column of Table 1a and/or Table 2 and/or an increase in expression and/or activity in the progenitor cell of one or more genes selected from Table 1b and/or a decrease in expression and/or activity in the progenitor cell of one or more genes selected from Table 1b. In some embodiments, the expression and/or activity of a gene is increased and/or decreased by increasing the expression of one or more transcription factors. Further, in this aspect, in some embodiments, altering the gene signature comprises an increase in expression and/or activity in the progenitor cell of a network module designated in the network module column of Table 1a and/or Table 1b. Further, in this aspect, in some embodiments, the activation of one or more genes of the network module designated in the network module column of Table 1a and/or Table 1b comprises modulating expression and/or activity of 2 or more genes within a network module. Further, in some embodiments of this aspect, altering the gene signature comprises an increase in expression and/or activity in the progenitor cell of two or more genes designated as an “up” gene in the gene directionality column of Table 1a. Further, in this aspect, in some embodiments, altering the gene signature comprises a decrease in expression and/or activity in the progenitor cell of two or more genes designated as a “down” gene in the gene directionality column of Table 1a. Further, in some embodiments of this aspect, altering the gene signature comprises an increase in expression and/or activity in the progenitor cell of two or more genes of Table 1b.

In another aspect, the present disclosure provides a method for making a therapeutic agent for a disease or disorder selected from a sickle cell disease, or a thalassemia, or a disease or disorder characterized by an abnormal oxygen delivery or a hemoglobin deficiency. In this aspect, the method comprises the steps of: (a) identifying a candidate perturbation for therapy as described above and (b) formulating the candidate perturbation as a therapeutic agent for the treatment of the disease or disorder.

An aspect of the present disclosure provides use of the perturbagen of Table 3, or a variant thereof in the manufacture of a medicament for treating a disease or disorder characterized by an abnormal oxygen delivery or a hemoglobin deficiency. In another aspect, the present disclosure provides use of the perturbagen of Table 3, or a variant thereof in the manufacture of a medicament for treating sickle cell disease or a thalassemia.

In some embodiments, the promoting the transition of a starting population of progenitor cells into erythrocytes comprising HbF or HbF-expressing progenitors thereof occurs in vitro or ex vivo. In an embodiment, promoting the transition of a starting population of progenitor cells into erythrocytes comprising HbF or HbF-expressing progenitors thereof occurs in vivo in a subject. In an embodiment, the subject is a human. In an embodiment, the human is an adult human.

The efficacy of the treatment with at least one perturbagen in the subject may be measured by an absolute increase or relative increase of HbF %, and or the F-cell percentage increase. % HbF is the proportion of HbF in the total Hb in hemolysate, which ignores the numbers of red blood cells. % F⁺ cells is the proportion of the HbF containing red blood cells in total red blood cells. (See Mundee et al., Cytometry (Communication in Clinical Cytometry), 2000, vol. 42, p. 389-393).

In some embodiments, the at least one perturbagen induces at least 50% absolute increase or a 100% relative increase in HbF percentage levels (HbF %). In some embodiments, the mean % HbF and % F⁺ cells in the subject treated with at least one perturbagen is about 13.0% or greater as measured by, e.g., HPLC and about 45.0% or greater as measured by, e.g., immunofluorescence technique respectively. In some embodiments, the mean % HbF in the subject treated with at least one perturbagen ranges from about 25.0% to about 30.0% as measured by, e.g., HPLC.

In some embodiments, the mean % HbF in the subject treated with at least one perturbagen is selected from about 4.0%, about 5.0%, about 6.0%, about 7.0%, about 8.0%, about 9.0%, about 10.0%, about 11.0%, about 12.0%, about 13.0%, about 13.1%, about 13.2%, about 13.3%, about 13.4%, about 13.5%, about 13.6%, about 13.7%, about 13.8%, about 13.9%, about 14.0%, about 14.1%, about 14.2%, about 14.3%, about 14.4%, about 14.5%, about 14.6%, about 14.7%, about 14.8%, about 14.9%, about 15.0%, about 15.1%, about 15.2%, about 15.3%, about 15.4%, about 15.5%, about 15.6%, about 15.7%, about 15.8%, about 15.9%, about 16.0%, about 16.1%, about 16.2%, about 16.3%, about 16.4%, about 16.5%, about 16.6%, about 16.7%, about 16.8%, about 16.9%, about 17.0%, about 17.1%, about 17.2%, about 17.3%, about 17.4%, about 17.5%, about 17.6%, about 17.7%, about 17.8%, about 17.9%, about 18.0%, about 18.1%, about 18.2%, about 18.3%, about 18.4%, about 18.5%, about 18.6%, about 18.7%, about 18.8%, about 18.9%, about 19.0%, about 18.1%, about 18.2%, about 18.3%, about 18.4%, about 18.5%, about 18.6%, about 18.7%, about 18.8%, about 18.9%, about 20.0%, about 20.1%, about 20.2%, about 20.3%, about 20.4%, about 20.5%, about 20.6%, about 20.7%, about 20.8%, about 20.9%, about 21.0%, about 21.1%, about 21.2%, about 21.3%, about 21.4%, about 21.5%, about 21.6%, about 21.7%, about 21.8%, about 21.9%, about 22.0%, about 22.1%, about 22.2%, about 22.3%, about 22.4%, about 22.5%, about 22.6%, about 22.7%, about 22.8%, about 22.9%, about 23.0%, about 23.1%, about 23.2%, about 23.3%, about 23.4%, about 23.5%, about 23.6%, about 23.7%, about 23.8%, about 23.9%, about 24.0%, about 24.1%, about 24.2%, about 24.3%, about 24.4%, about 24.5%, about 24.6%, about 24.7%, about 24.8%, about 24.9%, about 25.0%, about 25.1%, about 25.2%, about 25.3%, about 25.4%, about 25.5%, about 25.6%, about 250.7%, about 25.8%, about 25.9%, about 26.0%, about 26.1%, about 26.2%, about 26.3%, about 26.4%, about 26.5%, about 26.6%, about 26.7%, about 26.8%, about 26.9%, about 27.0%, about 27.1%, about 27.2%, about 27.3%, about 27.4%, about 27.5%, about 27.6%, about 27.7%, about 27.8%, about 27.9%, about 28.0%, about 28.1%, about 28.2%, about 28.3%, about 28.4%, about 28.5%, about 28.6%, about 28.7%, about 28.8%, about 28.9%, about 29.0%, about 29.1%, about 29.2%, about 29.3%, about 29.4%, about 29.5%, about 29.6%, about 29.7%, about 29.8%, about 29.9%, and about 30.0% as measured by e.g., HPLC.

In some embodiments, the mean % HbF in the subject treated with at least one perturbagen is selected from about 25.0%, about 25.1%, about 25.2%, about 25.3%, about 25.4%, about 25.5%, about 25.6%, about 250.7%, about 25.8%, about 25.9%, about 26.0%, about 26.1%, about 26.2%, about 26.3%, about 26.4%, about 26.5%, about 26.6%, about 26.7%, about 26.8%, about 26.9%, about 27.0%, about 27.1%, about 27.2%, about 27.3%, about 27.4%, about 27.5%, about 27.6%, about 27.7%, about 27.8%, about 27.9%, about 28.0%, about 28.1%, about 28.2%, about 28.3%, about 28.4%, about 28.5%, about 28.6%, about 28.7%, about 28.8%, about 28.9%, about 29.0%, about 29.1%, about 29.2%, about 29.3%, about 29.4%, about 29.5%, about 29.6%, about 29.7%, about 29.8%, about 29.9%, and about 30.0% as measured by e.g., HPLC.

In some embodiments, the treatment with at least one perturbagen causes a median % F⁺ cells increase ranges about 0.1% to about 50% above the baseline % F″ cells as measured by immunofluorescence technique. In some embodiments, the treatment with at least one perturbagen causes a median % F″ cells increase selected from the group consisting of about 0.1%, about 0.5%, about 1.0%, about 2.0%, about 3.0%, about 4.0%, about 5.0%, about 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0%, about 20.0%, about 21.0%, about 22.0%, about 23.0%, about 24.0%, about 25.0%, about 26.0%, about 27.0%, about 28.0%, about 29.0%, about 30.0%, about 31.0%, about 32.0%, about 33.0%, about 34.0%, about 35.0%, about 36.0%, about 37.0%, about 38.0%, about 39.0%, about 40.0%, about 41.0%, about 42.0%, about 43.0%, about 44.0%, about 45.0%, about 46.0%, about 47.0%, about 48.0%, about 49.0%, and about 50.0% as measured by e.g., HPLC.

Any aspect or embodiment disclosed herein can be combined with any other aspect or embodiment as disclosed herein.

### Administration, Dosing, and Treatment Regimens

As examples, administration results in the delivery of one or more perturbagens disclosed herein into the bloodstream (via enteral or parenteral administration), or alternatively, the one or more perturbagens is administered directly to the site of hematopoietic cell proliferation and/or maturation, i.e., in the bone marrow.

Delivery of one or more perturbagens disclosed herein to the bone marrow may be via intravenous injection or intravenous infusion or via intraosseous injection or intraosseous infusion. Devices and apparatuses for performing these delivery methods are well known in the art.

Delivery of one or more perturbagens disclosed herein into the bloodstream via intravenous injection or intravenous infusion may follow or be contemporaneous with stem cell mobilization. In stem cell mobilization, certain drugs are used to cause the movement of stem cells from the bone marrow into the bloodstream. In embodiments, once in the bloodstream, the stem cells are contacted with the one or more perturbagens. In embodiments, once in the bloodstream, the stem cells are contacted with the one or more perturbagens and are able to alter a gene signature in a progenitor cell, for example. Drugs and methods relevant to stem cell mobilization are well known in the art; see, e.g., Mohammadi et al, “Optimizing Stem Cells Mobilization Strategies to Ameliorate Patient Outcomes: A Review of Guide-lines and Recommendations.” Int. J. Hematol. Oncol. Stem Cell Res. 2017 Jan. 1; 11(1): 78-88; Hopman and DiPersio “Advances in Stem Cell Mobilization.” Blood Review, 2014, 28(1): 31-40; and Kim “Hematopoietic stem cell mobilization: current status and future perspective.” Blood Res. 2017 June; 52(2): 79-81. The content of each of which is incorporated herein by reference in its entirety.

Dosage forms suitable for parenteral administration include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any perturbagen disclosed herein as well as the dosing schedule can depend on various parameters and factors, including, but not limited to, the specific perturbagen, the disease being treated, the severity of the condition, whether the condition is to be treated or prevented, the subject's age, weight, and general health, and the administering physician's discretion. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In another embodiment, delivery can be in a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989).

A perturbagen disclosed herein can be administered by a controlled-release or a sustained-release means or by delivery a device that is well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydroxypropyl methylcellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, e.g., the bone marrow, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533 may be used.

The dosage regimen utilizing any perturbagen disclosed herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the disclosure employed.

Any perturbagen disclosed herein can be administered in a single daily dose (also known as QD, qd or q. d.), or the total daily dosage can be administered in divided doses of twice daily (also known as BID, bid, or b.i.d.), three times daily (also known as TID, tid, or t.i.d.), or four times daily (also known as QID, qid, or q.i.d.). Furthermore, any perturbagen disclosed herein can be administered continuously rather than intermittently throughout the dosage regimen.

### Pharmaceutical Compositions and Formulations

Aspects of the present disclosure include a pharmaceutical composition comprising a therapeutically effective amount of one or more perturbagens, as disclosed herein.

The perturbagens disclosed herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety. In some embodiments, the compositions disclosed herein are in the form of a pharmaceutically acceptable salt.

Further, any perturbagen disclosed herein can be administered to a subject as a component of a composition, e.g., pharmaceutical composition that comprises a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In some embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any perturbagen disclosed herein, if desired, can also formulated with wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

In some embodiments, the compositions, e.g., pharmaceutical compositions, disclosed herein are suspended in a saline buffer (including, without limitation TBS, PBS, and the like).

The present disclosure includes the disclosed perturbagens in various formulations of pharmaceutical compositions. Any perturbagens disclosed herein can take the form of solutions, suspensions, emulsion, drops, tablets, pills, pellets, capsules, capsules containing liquids, powders, sustained-release formulations, emulsions, aerosols, sprays, suspensions, or any other form suitable for use.

Where necessary, the pharmaceutical compositions comprising the perturbagens can also include a solubilizing agent. Also, the agents can be delivered with a suitable vehicle or delivery device as known in the art.

In some embodiments, the present disclosure provides a pharmaceutical composition comprising the combination of two or more perturbagens selected from Table 3 for the treatment of a disease or disorder selected from the group consisting of sickle cell disease, thalassemia, disease or disorder characterized by an abnormal oxygen delivery, and hemoglobin deficiency.

In some embodiments, the present disclosure provides a pharmaceutical composition comprising the combination of two or more perturbagens, each with a different mechanism of action for the treatment of a disease or disorder selected from the group consisting of sickle cell disease, thalassemia, disease or disorder characterized by an abnormal oxygen delivery, and hemoglobin deficiency. In some embodiments, the present disclosure provides a pharmaceutical composition comprising the combination of two or more perturbagens, each with a different mechanism of action, selected from Table 3 for the treatment a disease or disorder selected from the group consisting of anemia, beta-thalassemia, sickle cell anemia (SS), sickle hemoglobin-C disease (SC), sickle beta-plus thalassemia, and sickle beta-zero thalassemia. In some embodiments, the present disclosure provides a pharmaceutical composition comprising the combination of two or more perturbagens, each with a different mechanism of action, selected from Table 3 for the treatment beta-thalassemia, or sickle cell anemia.

In some embodiments, the present disclosure provides a pharmaceutical composition comprising the combination of hydroxyurea and one or more perturbagens selected from Table 3.

In some embodiments, the present disclosure provides a pharmaceutical composition comprising the combination of two or more perturbagens, each with a different mechanism of action, selected from Table 3 for the treatment of a disease or disorder selected from the group consisting of sickle cell disease, thalassemia, disease or disorder characterized by an abnormal oxygen delivery, and hemoglobin deficiency. In some embodiments, the present disclosure provides a pharmaceutical composition comprising the combination of two or more perturbagens, each with a different mechanism of action, selected from Table 3 for the treatment a disease or disorder selected from the group consisting of anemia, beta-thalassemia, sickle cell anemia (SS), sickle hemoglobin-C disease (SC), sickle beta-plus thalassemia, and sickle beta-zero thalassemia. In some embodiments, the present disclosure provides a pharmaceutical composition comprising the combination of two or more perturbagens, each with a different mechanism of action, selected from Table 3 for the treatment beta-thalassemia, or sickle cell anemia.

In some embodiments, the present disclosure provides a pharmaceutical composition comprising the combination of hydroxyurea and one or more perturbagens selected from Table 3 for the treatment of a disease or disorder selected from the group consisting of sickle cell disease, thalassemia, disease or disorder characterized by an abnormal oxygen delivery, and hemoglobin deficiency. In some embodiments, the present disclosure provides a pharmaceutical composition comprising the combination of hydroxyurea and one or more perturbagens selected from Table 3 for the treatment a disease or disorder selected from the group consisting of anemia, beta-thalassemia, sickle cell anemia (SS), sickle hemoglobin-C disease (SC), sickle beta-plus thalassemia, and sickle beta-zero thalassemia. In some embodiments, the present disclosure provides a pharmaceutical composition comprising the combination of hydroxyurea and one or more perturbagens selected from Table 3 for the treatment beta-thalassemia, or sickle cell anemia.

In some embodiments, the present disclosure provides a pharmaceutical composition comprising the combination of hydroxyurea and one or more HDAC inhibitors selected from Table 3. In some embodiments, the present disclosure provides a pharmaceutical composition comprising the combination of hydroxyurea and trichostatin-a.

In some embodiments, the present disclosure provides a pharmaceutical composition comprising the combination of hydroxyurea and a FABI inhibitor disclosed in Table 3. In some embodiments, the present disclosure provides a pharmaceutical composition comprising the combination of hydroxyurea and isoniazid.

In some embodiments, the present disclosure provides a pharmaceutical composition comprising the combination of hydroxyurea and a CFTR channel potentiator disclosed in Table 3. In some embodiments, the present disclosure provides a pharmaceutical composition comprising the combination of hydroxyurea and ivacaftor.

In some embodiments, two or more perturbagens selected from Table 3 may be mixed into a single preparation or two or more perturbagens of the combination may be formulated into separate preparations for use in combination separately or at the same time. In some embodiments, the present disclosure provides a kit containing the two or more perturbagens selected from Table 3 of the combination, formulated into separate preparations. In some embodiments, the combination therapies, comprising more than one perturbagen, can be co-delivered in a single delivery vehicle or delivery device.

As used herein, the term “combination” or “pharmaceutical combination” refers to the combined administration of the perturbagens. The combination of two or more perturbagen may be formulated as fixed dose combination or co-packaged discrete perturbagen dosages. In some embodiments, the fixed dose combination therapy of perturbagens comprises bilayer tablet, triple layer tablet, multilayered tablet, or capsule having plurality populations of particles of perturbagens. In some embodiments, the combination of two or more perturbagens may be administered to a subject in need thereof, e.g., concurrently or sequentially.

In some embodiments, the combination therapies of perturbagens as described above give synergistic effects on induction of HbF in a subject. The term “synergistic,” or “synergistic effect” or “synergism” as used herein, generally refers to an effect such that the one or more effects of the combination of compositions is greater than the one or more effects of each component alone, or they can be greater than the sum of the one or more effects of each component alone. The synergistic effect can be greater than about 10%, 20%, 30%, 40%, 50%, 60%, 75%, 100%, 110%, 120%, 150%, 200%, 250%, 350%, or 500% or more than the effect on a subject with one of the components alone, or the additive effects of each of the components when administered individually. The effect can be any of the measurable effects described herein. Advantageously, such synergy between the agents when combined, may allow for the use of smaller doses of one or both agents, may provide greater efficacy at the same doses, and may prevent or delay the build-up of multi-drug resistance. The combination index (CI) method of Chou and Talalay may be used to determine the synergy, additive or antagonism effect of the agents used in combination (Chou, Cancer Res. 2010, vol. 70, pp. 440-446). When the CI value is less than 1, there is synergy between the compounds used in the combination; when the CI value is equal to 1, there is an additive effect between the compounds used in the combination and when CI value is more than 1, there is an antagonistic effect. The synergistic effect may be attained by co-formulating the agents of the pharmaceutical combination. The synergistic effect may be attained by administering two or more agents as separate formulations administered simultaneously or sequentially.

Compositions for administration can optionally include a local anesthetic such as, for example, lignocaine to lessen pain at the site of the injection.

The pharmaceutical compositions comprising the perturbagens of the present disclosure may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In some embodiments, any perturbagens disclosed herein is formulated in accordance with routine procedures as a pharmaceutical composition adapted for a mode of administration disclosed herein.

### OTHER ASPECTS OF THE PRESENT DISCLOSURE

Embodiments associated with any of the above-disclosed aspects are likewise relevant to the below-mentioned aspects. In other words, each of the embodiment mentioned above for the above aspects may be revised/adapted to be applicable to the below aspects.

Yet another aspect of the present disclosure is a use of the perturbagen of Table 3, or a variant thereof in the manufacture of a medicament for treating a disease or disorder characterized by an abnormal oxygen delivery, hemoglobin deficiency, major β-globin, sickle cell disease, and thalassemia.

In another aspect, the present disclosure provides a method of identifying a candidate perturbation for promoting the transition of a starting population of progenitor cells into erythrocytes and/or reticulocytes or progenitors thereof. The method includes the steps of: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of cells in the population of progenitor cells into erythrocytes and/or reticulocytes or progenitors thereof following exposure of the population of cells to the perturbation; and identifying the perturbation as a candidate perturbation for promoting the transition of a population of progenitor cells into erythrocytes and/or reticulocytes or progenitors thereof based on the perturbation signature. In this aspect, the perturbation signature is an increase in expression and/or activity in the progenitor cell of one or more genes selected from the genes designated as an “up” gene in the gene directionality column of Table 1a and/or Table 2, and/or a decrease in expression and/or activity in the progenitor cell of one or more genes selected from the genes designated as a “down” gene in the gene directionality column of Table 1a and/or Table 2 and/or an increase in expression and/or activity in the progenitor cell of one or more genes selected from Table 1b and/or a decrease in expression and/or activity in the progenitor cell of one or more genes selected from Table 1b. In some embodiments, the expression and/or activity of a gene is increased and/or decreased by increasing the expression of one or more transcription factors.

In another aspect, the present disclosure provides a method for making a therapeutic agent for a disease or disorder selected from sickle cell disease, thalassemia, disease or disorder characterized by an abnormal oxygen delivery, and hemoglobin deficiency. In another aspect, the present disclosure provides a method for making a therapeutic agent for a disease or disorder selected from the group consisting of anemia, beta-thalassemia, sickle cell anemia (SS), sickle hemoglobin-C disease (SC), sickle beta-plus thalassemia, and sickle beta-zero thalassemia. In another aspect, the present disclosure provides a method for making a therapeutic agent for sickle cell disease, or thalassemia. In embodiments, the method includes the steps of: (a) identifying a candidate perturbation for therapy; and (b) formulating the candidate perturbation as a therapeutic agent for the treatment of the disease or disorder. In this aspect, identifying a therapeutic agent for therapy comprises steps of: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell fate of the population of the population of progenitor cells into erythrocytes and/or reticulocytes or progenitors thereof following exposure of the population of cells to the perturbation; and identifying the perturbation as a candidate perturbation for promoting the transition of a population of progenitor cells into erythrocytes and/or reticulocytes or progenitors thereof based on the perturbation signature. Further, in some embodiments of this aspect, the perturbation signature is an increase in expression and/or activity in the progenitor cell of one or more genes selected from the genes designated as an “up” gene in the gene directionality column of Table 1a and/or Table 2, and/or a decrease in expression and/or activity in the progenitor cell of one or more genes selected from the genes designated as a “down” gene in the gene directionality column of Table 1a and/or Table 2 and/or an increase in expression and/or activity in the progenitor cell of one or more genes selected from Table 1b and/or a decrease in expression and/or activity in the progenitor cell of one or more genes selected from Table 1b. In some embodiments, the expression and/or activity of a gene is increased and/or decreased by increasing the expression of one or more transcription factors.

In various embodiments, the present methods reduce the amount of cells having sickling hemoglobin (HbS). In various embodiments, the present methods increase the amount of cells anti-sickling hemoglobin (HbF).

In various embodiments, the present methods involving the monitoring of cell sickling, e.g. with one or more in vitro sickling assays (see Example 3 and Smith et al. Variable deformability of irreversibly sickled erythrocytes. Blood. 1981; 58(1):71-78; van Beers et al. Imaging flow cytometry for automated detection of hypoxia-induced erythrocyte shape change in sickle cell disease Am J Hematol. 2014 June; 89(6): 598-603; and Rab, et al. Rapid and reproducible characterization of sickling during automated deoxygenation in sickle cell disease patients Am J Hematol. 2019 May; 94(5): 575-584.

To further evaluate the impact of the perturbagens in reducing disease burden, are performed on sickle derived erythrocytes cells by enrichment of enucleated erythrocytes followed by incubation of cells at low oxygen or incubation in 2% sodium metabisulfite. Cell sickling is monitored using time lapse imaging.

Yet another aspect of the present disclosure is a perturbagen capable of causing a change in a gene signature.

In an aspect, the present disclosure provides a perturbagen capable of causing a change in cell fate.

In another aspect, the present disclosure provides a perturbagen capable of causing a change in a gene signature and a change in cell fate.

In yet another aspect, the present disclosure provides a pharmaceutical composition comprising any herein disclosed perturbagen.

In a further aspect, the present disclosure provides a unit dosage form comprising an effective amount of the pharmaceutical composition comprising any herein disclosed perturbagen.

### Methods of Culturing Cells In Vitro to Perform Single-Cell Analyses

In carrying out the techniques described herein for identifying the causes of cell fate, it is useful to generate datasets regarding cellular-component measurements obtained from single-cells. To generate these datasets, a population of cells of interest may be cultured in vitro. Alternately, these datasets may be generated, from single cells that have not been previously cultured; for example, cells used in single cell analyses may be obtained from dissociated primary tissue or from a blood product. This latter method of generating datasets is often desirable if one wants to capture information of the primary cell/organ as close to the in vivo setting as possible. However, for cells undergoing culturing, single-cell measurements of one or more cellular-components of interest may be performed at one or more time periods during the culturing to generate datasets.

In some embodiments, cellular-components of interest include nucleic acids, including DNA, modified (e.g., methylated) DNA, RNA, including coding (e.g., mRNAs) or non-coding RNA (e.g., sncRNAs), proteins, including post-transcriptionally modified protein (e.g., phosphorylated, glycosylated, myristilated, etc. proteins), lipids, carbohydrates, nucleotides (e.g., adenosine triphosphate (ATP), adenosine diphosphate (ADP) and adenosine monophosphate (AMP)) including cyclic nucleotides such as cyclic adenosine monophosphate (cAMP) and cyclic guanosine monophosphate (cGMP), other small molecule cellular-components such as oxidized and reduced forms of nicotinamide adenine dinucleotide (NADP/NADPH), and any combinations thereof. In some embodiments, the cellular-component measurements comprise gene expression measurements, such as RNA levels.

Any one of a number of single-cell cellular-component expression measurement techniques may be used to collect the datasets. Examples include, but are not limited to single-cell ribonucleic acid (RNA) sequencing (scRNA-seq), scTag-seq, single-cell assay for transposase-accessible chromatin using sequencing (scATAC-seq), CyTOF/SCoP, E-MS/Abseq, miRNA-seq, CITE-seq, and so on. The cellular-component expression measurement can be selected based on the desired cellular-component to be measured. For instance, scRNA-seq, scTag-seq, and miRNA-seq measure RNA expression. Specifically, scRNA-seq measures expression of RNA transcripts, scTag-seq allows detection of rare mRNA species, and miRNA-seq measures expression of micro-RNAs. CyTOF/SCoP and E-MS/Abseq measure protein expression in the cell. CITE-seq simultaneously measures both gene expression and protein expression in the cell. And scATAC-seq measures chromatin conformation in the cell. Table 4 below provides links to example protocols for performing each of the single-cell cellular-component expression measurement techniques described herein.

TABLE 4
Example Measurement Protocols

| Technique | Protocol |
|---|---|
| RNA-seq | Olsen and Baryawno “Introduction to Single-Cell RNA Sequencing” Current |
| | Protocols in Molecular Biology. Volume 122, Issue 1, April 2018, e57 |
| Tag-seq | Rozenberg et al., “Digital gene expression analysis with sample multiplexing and |
| | PCR duplicate detection: A straightforward protocol”, BioTechniques, vol. 61, |
| | No. 1, March 2018 |
| ATAC-seq | Buenrostro et al., “ATAC-seq: A Method for Assaying Chromatin Accessibility |
| | Genome-Wide”, Curr Protoc Mol Biol. 2015; 109: 21.29.1-21.29.9 |
| miRNA-seq | Faridani et al., “Sinqle-cell sequencing of the small-RNA transcriptome” Nature |
| | Biotechnology volume 34, pages 1264-1266 (2016) |
| CyTOF/SCoPE-MS/Abseq | Bandura et al., “Mass Cytometry: Technique for Real Time Single Cell |
| | Multitarget Immunoassay Based on Inductively Coupled Plasma Time-of-Flight |
| | Mass Spectrometry”, Anal Chem. 2009 Aug. 15: 81(16): 6813-22 |
| | Shahi et al., “Abseq: Ultrahigh-throughput single cell protein profiling with droplet |
| | microfluidic barcoding”, Scientific Reports volume 7, Article number: 44447 |
| | (2017) |
| | Budnik et al., “SCoPE-MS: mass spectrometry of sinqle mammalian cells |
| | quantifies proteome heterogeneity during cell differentiation”, Genome Biology |
| | 2018 19: 161 |
| CITE-seq | Stoeckius et al., “Simultaneous epitope and transcriptome measurement in |
| | single cells”, Nature Methods, vol 14, pages 865-868 (2017) |

The cellular-component expression measurement technique used may result in cell death. Alternatively, cellular-components may be measured by extracting out of the live cell, for example by extracting cell cytoplasm without killing the cell. Techniques of this variety allow the same cell to be measured at multiple different points in time.

If the cell population is heterogeneous such that multiple different cell types that originate from a same “progenitor” cell are present in the population, then single-cell cellular-component expression measurements can be performed at a single time point or at relatively few time points as the cells grow in culture. As a result of the heterogeneity of the cell population, the collected datasets will represent cells of various types along a trajectory of transition.

If the cell population is substantially homogeneous such that only a single or relatively few cell types, mostly the “progenitor” cell of interest, are present in the population, then single-cell cellular-component expression measurements can be performed multiple times over a period of time as the cells transition.

A separate single-cell cellular-component expression dataset is generated for each cell, and where applicable at each of the time periods. The collection of single-cell cellular-component expression measurements from a population of cells at multiple different points in time can collectively be interpreted as a “pseudo-time” representation of cell expression over time for the cell types originating from the same “progenitor” cell. The term pseudo-time is used in two respects, first, in that cell state transition is not necessarily the same from cell to cell, and thus the population of cell provides a distribution of what transition processes a cell of that “progenitor” type is likely to go through over time, and second, that the cellular-component expression measurements of those multiple cell's expressions at multiple time points simulates the possible transition behavior over time, even if cellular-component expression measurements of distinct cells give rise to the datasets. As a deliberately simple example, even if cell X gave a dataset for time point A and cell Y gave a dataset for time point B, together these two datasets represent the pseudo-time of transition between time point A and time point B.

For convenience of description, two such datasets captured for a “same” cell at two different time periods (assuming a technique is used that does not kill the cell) are herein referred to as different “cells” (and corresponding different datasets) because in practice such cells will often be slightly or significantly transitioned from each other, in some cases having an entirely distinct cell type as determined from the relative quantities of various cellular-components. Viewed from this context, these two measurements of a single-cell at different time points can be interpreted as different cells for the purpose of analysis because the cell itself has changed.

Note that the separation of datasets by cell/time period described herein is for clarity of description, in practice, these datasets may be stored in computer memory and logically operated on as one or more aggregate dataset/s (e.g., by cell for all time periods, for all cells and time periods at once).

In some instances, it is useful to collect datasets where a “progenitor” cell of interest has been perturbed from its base line state. There are a number of possible reasons to do this, for example, to knock out one or more cellular-components, to evaluate the difference between healthy and diseased cell states. In these instances, a process may also include steps for introducing the desired modifications to the cells. For example, one or more perturbations may be introduced to the cells, tailored viruses designed to knock out one or more cellular-components may be introduced, clustered regularly interspaced short palindromic repeats (CRISPR) may be used to edit cellular-components, and so on. Examples of techniques that could be used include, but are not limited to, RNA interference (RNAi), Transcription activator-like effector nuclease (TALEN) or Zinc Finger Nuclease (ZFN).

Depending upon how the perturbation is applied, not all cells will be perturbed in the same way. For example, if a virus is introduced to knockout a particular gene, that virus may not affect all cells in the population. More generally, this property can be used advantageously to evaluate the effect of many different perturbations with respect to a single population. For example, a large number of tailored viruses may be introduced, each of which performs a different perturbation such as causing a different gene to be knocked out. The viruses will variously infect some subset of the various cells, knocking out the gene of interest. Single-cell sequencing or another technique can then be used to identify which viruses affected which cells. The resulting differing single-cell sequencing datasets can then be evaluated to identify the effect of gene knockout on gene expression in accordance with the methods described elsewhere in this description.

Other types of multi-perturbation cell modifications can be performed similarly, such as the introduction of multiple different perturbations, barcoding CRISPR, etc. Further, more than one type perturbation may be introduced into a population of cells to be analyzed. For example, cells may be affected differently (e.g., different viruses introduced), and different perturbations may be introduced into different sub-populations of cells.

Additionally, different subsets of the population of cells may be perturbed in different ways beyond simply mixing many perturbations and post-hoc evaluating which cells were affected by which perturbations. For example, if the population of cells is physically divided into different wells of a multi-well plate, then different perturbations may be applied to each well. Other ways of accomplishing different perturbations for different cells are also possible.

Below, methods are exemplified using single-cell gene expression measurements. It is to be understood that this is by way of illustration and not limitation, as the present disclosure encompasses analogous methods using measurements of other cellular-components obtained from single-cells. It is to be further understood that the present disclosure encompasses methods using measurements obtained directly from experimental work carried out by an individual or organization practicing the methods described in this disclosure, as well as methods using measurements obtained indirectly, e.g., from reports of results of experimental work carried out by others and made available through any means or mechanism, including data reported in third-party publications, databases, assays carried out by contractors, or other sources of suitable input data useful for practicing the disclosed methods.

As discussed herein, gene expression in a cell can be measured by sequencing the cell and then counting the quantity of each gene transcript identified during the sequencing. In some embodiments, the gene transcripts sequenced and quantified may comprise RNA, for example mRNA. In alternative embodiments, the gene transcripts sequenced and quantified may comprise a downstream product of mRNA, for example a protein such as a transcription factor. In general, as used herein, the term “gene transcript” may be used to denote any downstream product of gene transcription or translation, including post-translational modification, and “gene expression” may be used to refer generally to any measure of gene transcripts.

Although the remainder of this description focuses on the analysis of gene transcripts and gene expression, all of the techniques described herein are equally applicable to any technique that obtains data on a single-cell basis regarding those cells. Examples include single-cell proteomics (protein expression), chromatin conformation (chromatin status), methylation, or other quantifiable epigenetic effects.

The following description provides an example general description for culturing a population of cells in vitro in order to carry out single-cell cellular-component expression measurement multiple time periods. Methods for culturing cells in vitro are known in the art. Those of skill in the art will also appreciate how this process could be modified for longer/shorter periods, for additional/fewer single-cell measurement steps, and so on.

In one embodiment, the process for culturing cells in a first cell state into cells in a second cell state includes one or more of the following steps:
Day 0: Thaw cells in the first cell state into a plate in a media suitable for growth of the cells.
Day 1: Seed cells in the first cell state into a multi-well plate. If applicable, perform additional steps to affect gene expression by cells. For example, simultaneously infect with one or more viruses to activate or knock out genes of interest.
   Perform gene expression measurement iteration t₁ for cells in the wells.
Day 1+l: Change media as needed if any additional processes are performed.
   If applicable, perform gene expression measurement iteration tₗ cells in the wells.
Day 1+m: Change media to media appropriate to support growth of cells in the second cell state.
   If applicable, perform gene expression measurement iteration tₘ for cells in the wells.
Days 1+n, o, p, etc.: Media change as needed to support further cell state transition from the first cell state to the second cell state. If applicable, perform additional steps to affect further transition from the first cell state to the second cell state. For example, add perturbations of interest to push cells towards the second cell state.
   If applicable, perform gene expression measurement iterations tₙ, tₒ, tₚ, etc., for cells in the wells.
Day q: Perform gene expression measurement iteration t_{q} for cells in the wells and in the second state.
Collect cells into a tube and stain in suspension with antibodies matched to genes/proteins of interest, thereby sorting/identifying cells without having to lyse/destroy them. This step also can identify surface proteins that might not be seen with as much resolution in the setting of the cytoplasm. Image with a cell imaging system such as the BD Celestra flow cytometer or similar instrument by acquiring the cells from each well or tube. Quantify of number of cells per well that are in the first cell state and the number of cells per well that are in the second cell state. These steps can be used with unfixed cells.

Any aspect or embodiment disclosed herein can be combined with any other aspect or embodiment as disclosed herein.

### Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

Any terms not directly defined herein shall be understood to have the meanings commonly associated with them as understood within the art of the disclosure. Certain terms are discussed herein to provide additional guidance to the practitioner in describing the compositions, the devices, the methods and the like of aspects of the disclosure and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the disclosure herein.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”. Likewise, the term “and/or” covers both “or” and “and”.

Unless specifically stated or obvious from context, as used herein, the term “about” is understood as within a range of normal tolerance in the art, for example within 2 standard deviations of the mean. About is understood to be within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

As used herein, the terms “cell fate” and “cell state” are interchangeable and synonymous.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

The term “ex vivo” refers to a medical procedure in which an organ, cells, or tissue are taken from a living body for a treatment or procedure, and then returned to the living body (See NCI Dictionary of Cancer Terms, https://www.cancer.gov/publications/dictionaries/cancer-terms/def/ex-vivo).

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “perturbation” in reference to a cell (e.g., a perturbation of a cell or a cellular perturbation) refers to any treatment of the cell with one or more active agents capable of causing a change in the cell's lineage or cell state (or in the lineage or cell state of the cell's progeny). These active agents can be referred to as “perturbagens.” In some embodiments, the perturbagen can comprise, e.g., a small molecule, a biologic, a protein, a protein combined with a small molecule, an antibody-drug conjugate (ADC), a nucleic acid, such as an siRNA or interfering RNA, a cDNA over-expressing wild-type and/or mutant shRNA, a cDNA over-expressing wild-type and/or mutant guide RNA (e.g., Cas9 system, Cas9-gRNA complex, or other gene editing system), or any combination of any of the foregoing. As used herein, a perturbagen classified as a “compound” may be a small molecule or a biologic. Also, a perturbagen classified as “overexpression of gene” may be cDNA over-expressing a wild-type gene or an mRNA encoding a wild-type gene. In some embodiments, an mRNA may comprise a modified nucleotide that promotes stability of the mRNA and/or reduces toxicity to a subject. Examples of modified nucleotides useful in the present disclosure include pseudouridine and 5-methylcytidine. Where a perturbagen is (or includes) a nucleic acid or protein described by reference to a particular sequence, it should be understood that variants with similar function and nucleic acid or amino acid identity are encompassed as well, e.g., variants with about: 1%, 2%, 3%, 4%, 5%, 6%, 7%, 8%, 9%, 10%, 11%, 12%, 13%, 14%, 15%, or more, variation, i.e., having about: 99%, 98%, 97%, 96%, 95%, 94%, 93%, 92%, 91%, 90%, 89%, 88%, 87%, 86%, or 85% identity to the reference sequence; e.g., in some embodiments, having, for example, at least: 1, 2, 3, 4, 5, 6, 7, 8, 9, 10, 11, 12, 13, 14, 15, 20, 25, 30, 35, 40, 45, 50, or more, substitutions.

The term “progenitor” in reference to a cell (e.g., a progenitor cell) refers to any cell that is capable of transitioning from one cell state to at least one other cell state. Thus, a progenitor can differentiate into one or more cell types and/or can expand into one or more types of cell populations.

As used herein, the term “red blood cells” refers to both reticulocytes and erythrocytes.

As used herein, the terms “treat,” “treatment,” and/or “treating” may refer to the management of a disease, disorder, or pathological condition, or symptom thereof with the intent to cure, ameliorate, stabilize, prevent, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition).

As used herein, the term “preventing” (also prophylaxis) refers to action taken to decrease the chance of getting a disease or condition.

The term “subject,” refers to an individual organism such as a human or an animal. In some embodiments, the subject is a mammal (e.g., a human, a non-human primate, or a non-human mammal), a vertebrate, a laboratory animal, a domesticated animal, an agricultural animal, or a companion animal. In some embodiments, the subject is a human (e.g., a human patient). In some embodiments, the subject is a rodent, a mouse, a rat, a hamster, a rabbit, a dog, a cat, a cow, a goat, a sheep, or a pig.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The disclosure will be further described in the following examples, which do not limit the scope of the disclosure described in the claims.

Embodiment 1: A method for directing a change in cell state of a progenitor cell comprising: contacting a population of cells comprising a progenitor cell with at least one perturbagen selected from Table 3, or a variant thereof, wherein the at least one perturbagen is capable of altering a gene signature in the progenitor cell; and wherein the progenitor cell is a non-lineage committed CD34+ cell.
Embodiment 2: A method for directing a change in cell state of a progenitor cell, comprising: contacting a population of cells comprising a progenitor cell with at least one perturbagen capable of altering a gene signature in the progenitor cell, wherein altering the gene signature comprises an increase in expression and/or activity in the progenitor cell of one or more genes selected from the genes designated as an “up” gene in the gene directionality column of Table 1a and/or Table 2 and/or a decrease in expression and/or activity in the progenitor cell of one or more genes selected from the genes designated as a “down” gene in the gene directionality column of Table 1a and/or Table 2 and/or an increase in expression and/or activity in the progenitor cell of one or more genes selected from Table 1b and/or a decrease in expression and/or activity in the progenitor cell of one or more genes selected from Table 1b and wherein the progenitor cell is a non-lineage committed CD34+ cell.
Embodiment 3: A method for directing a change in cell state of a progenitor cell, comprising: contacting a population of cells comprising a progenitor cell with at least one perturbagen selected from Table 3, or a variant thereof, and capable of altering a gene signature in the progenitor cell, wherein altering the gene signature comprises an increase in expression and/or activity in the progenitor cell of one or more genes selected from the genes designated as an “up” gene in the gene directionality column of Table 1a and/or Table 2 and/or a decrease in expression and/or activity in the progenitor cell of one or more genes selected from the genes designated as a “down” gene in the gene directionality column of Table 1a and/or Table 2 and/or an increase in expression and/or activity in the progenitor cell of one or more genes selected from Table 1b and/or a decrease in expression and/or activity in the progenitor cell of one or more genes selected from Table 1b and wherein the progenitor cell is a non-lineage committed CD34+ cell.
Embodiment 4: The method of Embodiment 2 or 3, wherein altering the gene signature comprises an increase in expression and/or activity in the progenitor cell of a network module designated in the network module column of Table 1a and/or Table 1b.
Embodiment 5: The method of Embodiment 4, wherein the activation of one or more genes of the network module designated in the network module column of Table 1a and/or Table 1b comprises modulating expression and/or activity of 2 or more genes within a network module.
Embodiment 6: The method of Embodiment 2 or 3, wherein altering the gene signature comprises an increase in expression and/or activity in the progenitor cell of two or more genes designated as an “up” gene in the gene directionality column of Table 1a and/or Table 2.
Embodiment 7: The method of Embodiment 6, wherein altering the gene signature comprises a decrease in expression and/or activity in the progenitor cell of two or more genes designated as a “down” gene in the gene directionality column of Table 1a and/or Table 2.
Embodiment 8: The method of any one of Embodiments 1 to 7, wherein the change in cell state provides an increase in the number of one or more of proerythroblasts, early erythroblasts, intermediate erythroblasts, late erythroblasts, reticulocytes, and erythrocytes, wherein one or more of the proerythroblasts, early erythroblasts, intermediate erythroblasts, late erythroblasts, reticulocytes, and erythrocytes expresses fetal hemoglobin (HbF).
Embodiment 9: The method of any one of Embodiments 1 to 8, wherein the change in cell state provides an increase in F cells.
Embodiment 10: The method of Embodiment 8 or 9, wherein one or more of the proerythroblasts, early erythroblasts, intermediate erythroblasts, late erythroblasts, reticulocytes, and erythrocytes expresses HBG1 and/or HBG2
Embodiment 11: The method of Embodiment 10, wherein the increase in the number of erythrocytes comprising HbF is relative to the number of erythrocytes obtained from a population of progenitor cells that is not contacted with the at least one perturbagen.
Embodiment 12: The method of Embodiment 10, wherein the increase in the number of erythrocytes comprising HbF is relative to the population of progenitor cells prior to contacting with the at least one perturbagen.
Embodiment 13: The method of Embodiment 11 or Embodiment 12, wherein the change in cell state provides an increase in the number of erythrocytes comprising HbF.
Embodiment 14: The method of Embodiment 8, wherein the ratio of the number of erythrocytes comprising HbF to the number of progenitor cells is increased relative to the ratio obtained from a population of progenitor cells that is not contacted with the at least one perturbagen.
Embodiment 15: The method of Embodiment 8, wherein the ratio of the number of erythrocytes comprising HbF to the number of progenitor cells is increased relative to the ratio in the population of progenitor cells prior to contacting with the at least one perturbagen.
Embodiment 16: The method of any one of Embodiments 8 to 15, wherein the increase in the number of erythrocytes comprising HbF, is due in part to increased cell proliferation of the erythrocytes comprising HbF.
Embodiment 17: The method of any one of Embodiments 8 to 16, wherein the increase in the number of erythrocytes comprising HbF, is due in part to an increased lifespan of the erythrocytes comprising HbF.
Embodiment 18: The method of any one of Embodiments 8 to 17, wherein the increase in the number of erythrocytes comprising HbF, is due in part to reduced cell death among the erythrocytes comprising HbF.
Embodiment 19: The method of any one of Embodiments 8 to 18, wherein the increase in the number of erythrocytes comprising HbF, is due in part to a change of cell state from progenitor cells into the erythrocyte lineage.
Embodiment 20: The method of any one of Embodiments 1 to 19, wherein the number of progenitor cells is decreased.
Embodiment 21: The method of Embodiment 20, wherein the decrease in the number of progenitor cells is due in part to decreased cell proliferation of the progenitor cells.
Embodiment 22: The method of Embodiment 20 or Embodiment 21, wherein the decrease in the number of progenitor cells is due in part to a decreased lifespan of the progenitor cells.
Embodiment 23: The method of any one of Embodiments 20 to 22, wherein the decrease in the number of progenitor cells is due in part to increased cell death among the progenitor cells.
Embodiment 24: The method of any one of Embodiments 20 to 23, wherein the decrease in the number of progenitor cells is relative to the number of progenitor cells in a population of progenitor cells that is not contacted with the at least one perturbagen.
Embodiment 25: The method of any one of Embodiments 20 to 24, wherein the decrease in the number of progenitor cells is relative to the number of progenitor cells in the population prior to contacting with the at least one perturbagen.
Embodiment 26: The method of any one of Embodiments 20 to 25, wherein the decrease in the number of progenitor cells is due to a change of cell state from a progenitor cell into the erythrocyte lineage.
Embodiment 27: The method of any one of Embodiments 1 to 19, wherein the number of progenitor cells is increased.
Embodiment 28: The method of Embodiment 27, wherein the increase in the number of progenitor cells is due in part to increased cell proliferation of the progenitor cells.
Embodiment 29: The method of Embodiment 27 or Embodiment 28, wherein the increase in the number of progenitor cells is due in part to an increased lifespan of the progenitor cells.
Embodiment 30: The method of any one of Embodiments 27 to 29, wherein the increase in the number of progenitor cells is due in part to decreased cell death among the progenitor cells.
Embodiment 31: The method of any one of Embodiments 27 to 30, wherein the increase in the number of progenitor cells is relative to the number of progenitor cells in a population of progenitor cells that is not contacted with the at least one perturbagen.
Embodiment 32: The method of any one of Embodiments 27 to 31, wherein the increase in the number of progenitor cells is relative to the number of progenitor cells in the population prior to contacting with the at least one perturbagen.
Embodiment 33: The method of any one of Embodiments 1 to 19, wherein the number of proerythroblasts, BFU-E cells, CFU-E cells, early erythroblasts, intermediate erythroblasts, late erythroblasts, reticulocytes, and/or erythrocytes is increased after contacting the population of cells comprising a CD34+ cell with the at least one perturbagen, wherein one or more of the proerythroblasts, early erythroblasts, intermediate erythroblasts, late erythroblasts, reticulocytes, and erythrocytes comprise HbF.
Embodiment 34: The method of any one of Embodiments 1 to 19, wherein the number of erythrocytes comprising HbF is increased after contacting the population of cells comprising a CD34+ cell with the at least one perturbagen.
Embodiment 35: The method of any one of Embodiments 1 to 19, wherein the number of reticulocytes comprising HbF, and/or erythrocytes comprising HbF is increased after contacting the population of cells comprising a CD34+ cell with the at least one perturbagen.
Embodiment 36: The method of Embodiment 33, wherein the ratio of the number of proerythroblasts, BFU-E cells, CFU-E cells, early erythroblasts, intermediate erythroblasts, late erythroblasts, reticulocytes, and/or erythrocytes to the number of progenitor cells is increased relative to the ratio obtained from a population of progenitor cells that is not contacted with the at least one perturbagen, wherein one or more of the proerythroblasts, early erythroblasts, intermediate erythroblasts, late erythroblasts, reticulocytes, and erythrocytes comprise HbF.
Embodiment 37: The method of Embodiment 33, wherein the ratio of the number proerythroblasts, BFU-E cells, CFU-E cells, early erythroblasts, intermediate erythroblasts, late erythroblasts, reticulocytes, and/or erythrocytes to the number of progenitor cells is increased relative to the ratio in the population of progenitor cells prior to contacting with the at least one perturbagen, wherein one or more of the proerythroblasts, early erythroblasts, intermediate erythroblasts, late erythroblasts, reticulocytes, and erythrocytes comprise HbF.
Embodiment 38: The method of any one of Embodiments 1 to 19, wherein the ratio of the number of early erythroblasts, intermediate erythroblasts, late erythroblasts, reticulocytes, and/or erythrocytes to the number of proerythroblasts cells is increased relative to the ratio obtained from a population of progenitor cells that is not contacted with the at least one perturbagen, wherein one or more of the proerythroblasts, early erythroblasts, intermediate erythroblasts, late erythroblasts, reticulocytes, and erythrocytes comprise HbF.
Embodiment 39: The method of any one of Embodiments 1 to 19, wherein the ratio of the number of early erythroblasts, intermediate erythroblasts, late erythroblasts, reticulocytes, and/or erythrocytes to the number of proerythroblasts is increased relative to the ratio in the population of progenitor cells prior to contacting with the at least one perturbagen, wherein one or more of the proerythroblasts, early erythroblasts, intermediate erythroblasts, late erythroblasts, reticulocytes, and erythrocytes comprise HbF.
Embodiment 40: The method of any one of Embodiments 1 to 19, wherein the ratio of the number of HBF-expressing early erythroblasts to the number of HBF-expressing proerythroblasts is increased relative to the ratio obtained from a population of progenitor cells that is not contacted with the at least one perturbagen.
Embodiment 41: The method of any one of Embodiments 1 to 19, wherein the ratio of the number of HBF-expressing early erythroblasts to the number of HBF-expressing proerythroblasts is increased relative to the ratio in the population of progenitor cells prior to contacting with the at least one perturbagen.
Embodiment 42: The method of any one of Embodiments 1 to 19, wherein the ratio of the number of HBF-expressing intermediate erythroblasts to the number of HBF-expressing early erythroblasts is increased relative to the ratio obtained from a population of progenitor cells that is not contacted with the at least one perturbagen.
Embodiment 43: The method of any one of Embodiments 1 to 19, wherein the ratio of the number of HBF-expressing intermediate erythroblasts to the number of HBF-expressing early erythroblasts is increased relative to the ratio in the population of progenitor cells prior to contacting with the at least one perturbagen.
Embodiment 44: The method of any one of Embodiments 1 to 19, wherein the ratio of the number of HBF-expressing late erythroblasts to the number of HBF-expressing intermediate erythroblasts is increased relative to the ratio obtained from a population of progenitor cells that is not contacted with the at least one perturbagen.
Embodiment 45: The method of any one of Embodiments 1 to 19, wherein the ratio of the number of HBF-expressing late erythroblasts to the number of HBF-expressing intermediate erythroblasts is increased relative to the ratio in the population of progenitor cells prior to contacting with the at least one perturbagen.
Embodiment 46: The method of any one of Embodiments 1 to 19, wherein the ratio of the number of r reticulocytes comprising HbF to the number of HBF-expressing late erythroblasts is increased relative to the ratio obtained from a population of progenitor cells that is not contacted with the at least one perturbagen.
Embodiment 47: The method of any one of Embodiments 1 to 19, wherein the ratio of the number of reticulocytes comprising HbF to the number of HBF-expressing late erythroblasts is increased relative to the ratio in the population of progenitor cells prior to contacting with the at least one perturbagen.
Embodiment 48: The method of any one of Embodiments 1 to 19, wherein the ratio of the number of erythrocytes comprising HbF to the number of reticulocytes comprising HbF is increased relative to the ratio obtained from a population of progenitor cells that is not contacted with the at least one perturbagen.
Embodiment 49: The method of any one of Embodiments 1 to 19, wherein the ratio of the number of erythrocytes comprising HbF to the number of reticulocytes comprising HbF is increased relative to the ratio in the population of progenitor cells prior to contacting with the at least one perturbagen.
Embodiment 50: The method of any one of Embodiments 1 to 19, wherein the ratio of the number of erythrocytes comprising HbF to HBF-expressing late erythroblasts is increased relative to the ratio obtained from a population of progenitor cells that is not contacted with the at least one perturbagen.
Embodiment 51: The method of any one of Embodiments 1 to 19, wherein the ratio of the number of erythrocytes comprising HbF to HBF-expressing late erythroblasts is increased relative to the ratio in the population of progenitor cells prior to contacting with the at least one perturbagen.
Embodiment 52: The method of any one of Embodiments 1 to 19, wherein the ratio of the number of erythrocytes comprising HbF to HBF-expressing intermediate erythroblasts is increased relative to the ratio obtained from a population of progenitor cells that is not contacted with the at least one perturbagen.
Embodiment 53: The method of any one of Embodiments 1 to 19, wherein the ratio of the number of erythrocytes comprising HbF to HBF-expressing intermediate erythroblasts is increased relative to the ratio in the population of progenitor cells prior to contacting with the at least one perturbagen.
Embodiment 54: The method of any one of Embodiments 1 to 19, wherein the ratio of the number of erythrocytes comprising HbF to HBF-expressing early erythroblasts is increased relative to the ratio obtained from a population of progenitor cells that is not contacted with the at least one perturbagen.
Embodiment 55: The method of any one of Embodiments 1 to 19, wherein the ratio of the number of erythrocytes comprising HbF to HBF-expressing early erythroblasts is increased relative to the ratio in the population of progenitor cells prior to contacting with the at least one perturbagen.
Embodiment 56: The method of any one of Embodiments 1 to 19, wherein the ratio of the number of erythrocytes comprising HbF to HBF-expressing proerythroblasts is increased relative to the ratio obtained from a population of progenitor cells that is not contacted with the at least one perturbagen.
Embodiment 57: The method of any one of Embodiments 1 to 19, wherein the ratio of the number of erythrocytes comprising HbF to HBF-expressing proerythroblasts is increased relative to the ratio in the population of progenitor cells prior to contacting with the at least one perturbagen.
Embodiment 58: The method of any one of Embodiments 1 to 19, wherein the ratio of the number of erythrocytes comprising HbF to proerythroblasts is increased relative to the ratio obtained from a population of progenitor cells that is not contacted with the at least one perturbagen.
Embodiment 59: The method of any one of Embodiments 1 to 19, wherein the ratio of the number of erythrocytes comprising HbF to proerythroblasts is increased relative to the ratio in the population of progenitor cells prior to contacting with the at least one perturbagen.
Embodiment 60: The method of any of Embodiments 1 to 59, wherein the number of proerythroblasts is decreased.
Embodiment 61: The method of any of Embodiments 1 to 59, wherein the number of HBF-negative or HBF-low proerythroblasts is decreased.
Embodiment 62: The method of any of Embodiments 1 to 59, wherein the number of HBF-negative or HBF-low early erythroblasts is decreased.
Embodiment 63: The method of any of Embodiments 1 to 59, wherein the number of HBF-negative or HBF-low intermediate erythroblasts is decreased.
Embodiment 64: The method of any of Embodiments 1 to 59, wherein the number of HBF-negative or HBF-low late erythroblasts is decreased.
Embodiment 65: The method of any of Embodiments 1 to 59, wherein the number of HBF-negative or HBF-low reticulocytes is decreased.
Embodiment 66: The method of any of Embodiments 1 to 59, wherein the number of proerythroblasts is increased.
Embodiment 67: The method of any of Embodiments 1 to 59, wherein the number of HBF-positive or HBF-high proerythroblasts is increased.
Embodiment 68: The method of any of Embodiments 1 to 59, wherein the number of HBF-positive or HBF-high early erythroblasts is increased.
Embodiment 69: The method of any of Embodiments 1 to 59, wherein the number of HBF-positive or HBF-high intermediate erythroblasts is increased.
Embodiment 70: The method of any of Embodiments 1 to 59, wherein the number of HBF-positive or HBF-high late erythroblasts is increased.
Embodiment 71: The method of any of Embodiments 1 to 59, wherein the number of HBF-positive or HBF-high reticulocytes is increased.
Embodiment 72: The method of any of Embodiments 1 to 59, wherein the number of HBF-positive or HBF-high erythrocytes is increased.
Embodiment 73: The method of any of Embodiments 1 to 59, wherein the number of F cells is increased.
Embodiment 74: The method of any of Embodiments 1 to 59, wherein the ratio of the number of F cells to non-F cells is increased relative to the ratio obtained from a population of progenitor cells that is not contacted with the at least one perturbagen.
Embodiment 75: The method of any of Embodiments 1 to 59, wherein the ratio of the number of F cells to non-F cells is increased relative to the ratio in the population of progenitor cells prior to contacting with the at least one perturbagen.
Embodiment 76: The method of any one of Embodiments 1 to 75, wherein the at least one perturbagen selected from Table 3, or a variant thereof, comprises at least 2, at least 3, at least 4, or at least 5 perturbagens selected from Table 3, or variants thereof.
Embodiment 77 The method of any one of Embodiments 1 to 76, wherein the at least one perturbagen is used in combination with one or more additional therapeutic agents.
Embodiment 78: The method of Embodiment 77, wherein the additional therapeutic agent is hydroxyurea (HU).
Embodiment 79: The method of Embodiment 2 or 3, wherein the one or more genes selected from the genes designated as an “up” gene in the gene directionality column of Table 1a comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, 81 or more, 82 or more, 83 or more, 84 or more, 85 or more, 86 or more, 87 or more, 88 or more, 89 or more, 90 or more, 91 or more, 92 or more, 93 or more, 94 or more, 95 or more, 96 or more, 97 or more, 98 or more, 99 or more, 100 or more, 101 or more, 102 or more, 103 or more, 104 or more, or 105 or more genes selected from the genes designated as an “up” gene in the gene directionality column of Table 1a.
Embodiment 80: The method of Embodiment 79, wherein the one or more genes selected from the genes designated as an “up” gene in the gene directionality column of Table 1a comprises at least one of DDIT4, EPRS, MTHFD2, EIF4EBP1, AARS, ABCC5, PHGDH, TUBB6, LSM6, EIF4G1, RNF167, CD320, CTNNAL1, GADD45A, PTK2, CFLAR, IGF2BP2, CDK1, CDC45, CDCA4, MELK, HAT1, PAK1, TSPAN6, TIMM17B, KDM5A, UBE3B, RPS5, PAICS, RPIA, KDELR2, PNP, CAST, H2AFV, ATP11B, CTNND1, ORC1, FDFT1, CDKN1B, INSIG1, IGF1R, TRAP1, TSTA3, SUZ12, CDK4, HMGCS1, LAP3, TBPL1, FAH, CCP110, APOE, IGF2R, DYRK3, MYBL2, APP, DNMT1, SMC3, HTATSF1, CAT, ACAT2, HK1, PSMD4, CLTC, MAP4K4, PROS1, DLD, SDHB, GNAS, COPS7A, MPC2, HEBP1, BLVRA, ID2, SCAND1, ETFB, MRPS16, PIN1, TRAK2, AMDHD2, PLEKHJ1, BZW2, PCNA, WDR61, RFC5, OXA1L, MCM3, CEP57, PSMF1, POLR2K, PSMD2, ATP6V1D, PSMD9, AKAP8L, GRN, SPAG7, ENOSF1, PCK2, PCCB, NOLC1, EBNA1BP2, CD58, RFC2, ASAH1, LAGE3, AKR7A2, and RSU1.
Embodiment 81: The method of Embodiment 2 or 3, wherein the one or more genes selected from the genes designated as a “down” gene in the gene directionality column of Table 1a comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, or 26 or more genes selected from the genes designated as a “down” gene in the gene directionality column of Table 1a.
Embodiment 82: The method of Embodiment 81, wherein the one or more genes selected from the genes designated as a “down” gene in the gene directionality column of Table 1a comprises at least one of NUCB2, XBP1, CCNB1, CDC20, PLK1, CDK6, ITGB1BP1, CCNE2, PTPN6, CBR1, HLA-DRA, MAP7, SOX4, CASP3, DNAJB6, HOXA10, IL1B, ICAM3, ADGRG1, HLA-DMA, PDLIM1, PSMB8, EPB41L2, RPL39L, PYGL, CYB561, and HOMER2.
Embodiment 83: The method of Embodiment 2 or 3, wherein the one or more genes selected from the genes designated as an “up” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, o 16 or more, 17 or more, 18 or more, or 19 or more genes selected from the genes designated as an “up” gene in the gene directionality column of Table 2.
Embodiment 84: The method of Embodiment 83, wherein the one or more genes selected from the genes designated as an “up” gene in the gene directionality column of Table 2 comprises at least one of HMGA1, KLF1, KLF6, SREBF1, NFE2, ARID3A, GFI1B, KLF13, MLXIP, E2F8, MYBL2, HSF1, GMEB1, NFX1, TGIF1, KLF3, SP1, CENPX, HES6, and LIN28B.
Embodiment 85: The method of Embodiment 2 or 3, wherein the one or more genes selected from the genes designated as a “down” gene in the gene directionality column of Table 2 comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, or 19 or more genes selected from the genes designated as a “down” gene in the gene directionality column of Table 2.
Embodiment 86: The method of Embodiment 85, wherein the one or more genes selected from the genes designated as a “down” gene in the gene directionality column of Table 2 comprises at least one of HOXA10, XBP1, SOX4, ZNF385D, NFIC, BATF, HHEX, RARG, KDM5B, ZFX, SPI1, TEAD4, SATB1, NFIX, PLAGL1, MEF2C, ZBTB1, HOXA9, THAP5, and ZFP57.
Embodiment 87: The method of Embodiment 2 or 3, wherein the one or more genes selected from Table 1b comprises 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more, or 21 or more, or 22 or more, or 23 or more, or 24 or more, or 25 or more, or 26 or more, or 27 or more, or 28 or more, or 29 or more, or 30 or more, or 31 or more, or 32 or more, or 33 or more, or 34 or more, or 35 or more, or 36 or more, or 37 or more, or 38 or more, or 39 or more, or 40 or more, or 41 or more, or 42 or more, or 43 or more, or 44 or more, or 45 or more, or 46 or more, or 47 or more, or 48 or more, or 49 or more, 50 or more, 51 or more, or 52 or more, or 53 or more, or 54 or more, or 55 or more, or 56 or more, or 57 or more, or 58 or more, or 59 or more, 60 or more, or 61 or more, or 62 or more, or 63 or more, or 64 or more, or 65 or more, or 66 or more, or 67 or more, or 68 or more, or 69 or more, 70 or more, or 71 or more, or 72 or more, or 73 or more, or 74 or more, or 75 or more, or 76 or more, or 77 or more, or 78 or more, or 79 or more, 80 or more, or 81 or more, or 82 or more, or 83 or more, or 84 or more, or 85 or more, or 86 or more, or 87 or more, or 88 or more, or 89 or more, 90 or more, or 91 or more, or 92 or more, or 93 or more, or 94 or more, or 95 or more, or 96 or more, or 97 or more, or 98 or more, or 99 or more, or 100 or more, or 101 or more, or 102 or more, or 103 or more, or 104 or more, or 105 or more, or 106 or more, or 107 or more, or 108 or more, or 109 or more, or 110 or more, or 111 or more, or 112 or more, or 113 or more, or 114 or more, or 115 or more, or 116 or more, or 117 or more, or 118 or more, or 119 or more, or 120 or more, or 121 or more, or 122 or more, or 123 or more, or 124 or more, or 125 or more, or 126 or more, or 127 or more, or 128 or more, or 129 or more, or 130 or more, or 131 or more, or 132 or more, 133 or more, or 134 or more, or 135 or more, or 136 or more, or 137 or more, or 138 or more, or 139 or more, or 140 or more, or 141 or more, or 142 or more, or 143 or more, or 144 or more, or 145 or more, or 146 or more, or 147 or more, or 148 or more, or 149 or more, 150 or more, 151 or more, or 152 or more, or 153 or more, or 154 or more, or 155 or more, or 156 or more, or 157 or more, or 158 or more, or 159 or more, 160 or more, or 161 or more, or 162 or more, or 163 or more, or 164 or more, or 165 or more, or 166 or more, or 167 or more, or 168 or more, or 169 or more, 170 or more, or 171 or more, or 172 or more, or 173 or more, or 174 or more, or 175 or more, or 176 or more, or 177 or more, or 178 or more, or 179 or more, 180 or more, or 181 or more, or 182 or more, or 183 or more, or 184 or more, or 185 or more, or 186 or more, or 187 or more, or 188 or more, or 189 or more, 190 or more, or 191 or more, or 192 or more, or 193 or more, or 194 or more, or 195 or more, or 196 or more, or 197 or more, or 198 or more, or 199 or more, or 200 or more, or 201 or more, or 202 or more, or 203 or more, or 204 or more, or 205 or more, or 206 or more, or 207 or more, or 208 or more, or 209 or more, or 210 or more, or 211 or more, or 212 or more, or 213 or more, or 214 or more, or 215 or more, or 216 or more, or 217 or more, or 218 or more, or 219 or more, or 220 or more, or 221 or more, or 222 or more, or 223 or more, or 224 or more, or 225 or more, or 226 or more, or 227 or more, or 228 or more, or 229 or more, or 230 or more, or 231 or more, or 232 or more, or 233 or more, or 234 or more, or 235 or more, or 236 or more, or 237 or more, or 238 or more, or 239 or more, or 240 or more, or 241 or more, or 242 or more, or 243 or more, or 244 or more, or 245 or more, or 246 or more, or 247 or more, or 248 or more, or 249 or more, 250 or more, 251 or more, or 252 or more, or 253 or more, or 254 or more, or 255 or more, or 256 or more, or 257 or more, or 258 or more, or 259 or more, 260 or more, or 261 or more, or 262 or more, or 263 or more, or 264 or more, or 265 or more, or 266 or more, or 267 or more, or 268 or more, or 269 or more, 270 or more, or 271 or more, or 272 or more genes selected from Table 1b.
Embodiment 88: The method of Embodiment 87, wherein the one or more genes selected from Table 1b comprises at least one of RAP1GAP, E2F2, RSRP1, RHD, RHCE, ERMAP, SLC2A1, CD58, SELENBP1, PPOX, NPL, ADIPOR1, BTG2, KLHDC8A, SDE2, GUK1, LBH, LTBP1, ZC3H6, TRAK2, STRADB, TMBIM1, DNAJB2, KAT2B, ABHD5, CPOX, RAB6B, PAQR9, SIAH2, NCEH1, KLF3, FRYL, MOB1B, HERC6, TSPAN5, GYPE, GYPB, FHDC1, CLCN3, ANKH, EPB41L4A, IRF1, CYSTM1, FAXDC2, TRIM10, TSPO2, CCND3, GTPBP2, GCLC, FOXO3, SERINC1, CITED2, JAZF1, MTURN, CD36, PNPLA8, BPGM, KDM7A, MFHAS1, CTSB, SLC25A37, BNIP3L, RNF19A, GRINA, HSF1, AQP3, CTSL, TMOD1, STOM, RXRA, OPTN, FRMD4A, STAM, MXI1, UROS, RIC8A, ILK, SOX6, CAT, YPEL4, UCP2, PPME1, ENDOD1, PTMS, CMAS, NFE2, KIF5A, RAB3IP, NUDT4, HECTD4, SDSL, RB1, PNP, DCAF11, ATP6V1D, DPF3, ZFYVE21, KIF26A, KLF13, CCNDBP1, EPB42, REXO5, ITFG1, TERF2IP, SLC7A5, EPN2,NATD1, PLEKHH3, SLC4A1, FAM117A, WIPI1, SMIM5, LPIN2, RIOK3, UBXN6, 2-Mar, JUNB, AKAP8L, UPK1A-AS1, PPP1R15A, GPCPD1, FAM210B, RBM38, TUBB1, ITSN1, GRAP2, WWC3, ALAS2, FAM122C, MOSPD1, SOX4, CLSTN1, PGM1, CD2, PHGDH, MLLT11, IFI16, FCER1A, RGS18, CD34, NENF, PLEK, IL1B, IGFBP7, INPP4B, BASP1, FYB1, CD74, SERPINB6, TUBB2B, LTB, LST1, AIF1, MPIG6B, HLA-DRA, HLA-DRB1, HLA-DMA, HLA-DPA1, HLA-DPB1, MAP7, CPVL, SCRN1, GNAI1, CYP3A4, PRKAG2, CLU, PVT1, ALDH1A1, NFIL3, DNM1, FAM69B, NPDC1, VIM, ARID5B, ZMIZ1, SESN2, GADD45A, DENND2D, FAM91A3P, S100A6, IER5, RGS16, PHLDA3, XPC, LXN, ZMAT3, CDKN1A, SESN1, PLIN2, RPS6, CDKN2A, ANKRD18A, LCN12, FAS, CTSD, HBBP1, DDB2, CTTN, SNORD15B, MDM2, PXMP2, PLEK2, RPS27L, LYRM1, RNF167, RNU4-34P, MYL4, FDXR, TNFSF9, CD70, GDF15, ECH1, BBC3, BAX, FTL, RPS5, ADA, GNAS, APOBEC3H, RHOC, CYP1B1, SUCNR1, TIPARP, HSD17B11, HLA-A, HLA-B, PSMB9, ASAH1, VPS28, HACD1, BGLT3, HBG1, HBG2, TRIM22, PRDX5, TSC22D1, RGS6, IFI27L2, B2M, ARID3A, RABAC1, and BEX1.
Embodiment 89: The method of any one of Embodiments 1 to 88, wherein contacting the population of progenitor cells occurs in vitro or ex vivo.
Embodiment 90: The method of any one of Embodiments 1 to 88, wherein contacting the population of progenitor cells occurs in vivo in a subject.
Embodiment 91: The method of Embodiment 90, wherein the subject is a human.
Embodiment 92: The method of Embodiment 91, wherein the human is an adult human.
Embodiment 93: A perturbagen for use in the method of any one of Embodiments 1 to 92.
Embodiment 94: A pharmaceutical composition comprising the perturbagen of Embodiment 93.
Embodiment 95: A method for treating a disease or disorder characterized by an abnormal oxygen delivery, comprising: (a) administering to a subject in need thereof a therapeutically effective amount of at least one perturbagen selected from Table 3, or a variant thereof, wherein the at least one perturbagen is capable of changing a gene signature in a progenitor cell or (b) administering to a subject in need thereof a cell, the cell having been contacted with at least one perturbagen selected from Table 3, or a variant thereof, wherein the at least one perturbagen is capable of changing a gene signature in a progenitor cell.
Embodiment 96: A method for treating a disease or disorder characterized by a hemoglobin deficiency, comprising: (a) administering to a subject in need thereof a therapeutically effective amount of at least one perturbagen selected from Table 3, or a variant thereof, wherein the at least one perturbagen is capable of changing a gene signature in a progenitor cell or (b) administering to a subject in need thereof a cell, the cell having been contacted with at least one perturbagen selected from Table 3, or a variant thereof, wherein the at least one perturbagen is capable of changing a gene signature in a progenitor cell.
Embodiment 97: A method for treating or preventing a sickle cell disease or a thalassemia, comprising: (a) administering to a subject in need thereof a therapeutically effective amount of at least one perturbagen selected from Table 3, or a variant thereof, wherein the at least one perturbagen is capable of changing a gene signature in a progenitor cell or (b) administering to a subject in need thereof a cell, the cell having been contacted with at least one perturbagen selected from Table 3, or a variant thereof, wherein the at least one perturbagen is capable of changing a gene signature in a progenitor cell.
Embodiment 98: The method of Embodiment 96, wherein the hemoglobin deficiency is an abnormal and/or reduced oxygen delivery functionality of hemoglobin, optionally resultant from mutations in one or more hemoglobin genes, the mutation optionally being in a HBB gene.
Embodiment 99: The method of any one of Embodiments 95 to 97, wherein the administering is directed to the bone marrow of the subject.
Embodiment 100: The method of Embodiment 99, wherein the administering is via intraosseous injection or intraosseous infusion.
Embodiment 101: The method of any one of Embodiments 95 to 100, wherein the administering the cell is via intravenous injection or intravenous infusion.
Embodiment 102: The method of any one of Embodiments 96 to 101, wherein the administering is simultaneously or sequentially to one or more mobilization agents.
Embodiment 103: The method of any one of Embodiments 95 to 102, wherein the disease or disorder characterized by an abnormal oxygen delivery and/or a hemoglobin deficiency is an anemia.
Embodiment 104: The method of Embodiment 95 to 103, wherein the sickle cell disease or a thalassemia is beta-thalassemia (transfusion dependent).
Embodiment 105: The method of any one of Embodiments 95 to 103, wherein the sickle cell disease or a thalassemia is beta-thalassemia major.
Embodiment 106: The method of any one of Embodiments 95 to 103, wherein the sickle cell disease or a thalassemia is beta-thalassemia intermedia.
Embodiment 107: The method of any one of Embodiments 95 to 103, wherein the sickle cell disease or a thalassemia is beta-thalassemia minor.
Embodiment 108: The method of any one of Embodiments 95 to 103, wherein the sickle cell disease or a thalassemia is sickle cell anemia (SS), sickle hemoglobin-C disease (SC), sickle beta-plus thalassemia and sickle beta-zero thalassemia.
Embodiment 109: The method of any one of Embodiments 95 to 108, wherein the at least one perturbagen is capable of changing a gene signature in a progenitor cell.
Embodiment 110: The method of any one of Embodiments 95 to 108, wherein the subject is selected by steps comprising: obtaining from the subject having the disease or disorder a sample of cells comprising a non-lineage committed CD34+ cell; and contacting the sample of cells with least one perturbagen selected from Table 3, or a variant thereof, wherein the at least one perturbagen alters a gene signature in the sample of cells.
Embodiment 111: The method of any one of Embodiments 95 to 108, wherein the subject is selected by steps comprising: obtaining from a subject having the disease or disorder a sample of cells comprising a non-lineage committed CD34+ cell; and contacting the sample of cells with at least one perturbagen capable of altering a gene signature in a non-lineage committed CD34+ cell, wherein the at least one perturbagen increases in the sample of cells the expression and/or activity of one or more genes selected from the genes designated as an “up” gene in the gene directionality column of Table 1a and/or Table 2 and/or decreases in the sample of cells the expression and/or activity of one or more genes selected from the genes designated as a “down” gene in the gene directionality column of Table 1a and/or Table 2 and/or increases in the sample of cells the expression and/or activity of one or more genes selected from Table 1b and/or decreases in the sample of cells the expression and/or activity of one or more genes selected from Table 1b.
Embodiment 112: The method of any one of Embodiments 95 to 108, wherein the subject is selected by steps comprising: obtaining from a subject having the disease or disorder a sample of cells comprising a non-lineage committed CD34+ cell; and contacting the sample of cells with at least one perturbagen selected from Table 3, or a variant thereof; wherein the at least one perturbagen increases in the sample of cells the expression and/or activity of one or more genes selected from the genes designated as an “up” gene in the gene directionality column of Table 1a and/or Table 2 and/or decreases in the sample of cells the expression and/or activity of one or more genes selected from the genes designated as a “down” gene in the gene directionality column of Table 1a and/or Table 2 and/or increases in the sample of cells the expression and/or activity of one or more genes selected from Table 1b and/or decreases in the sample of cells the expression and/or activity of one or more genes selected from Table 1b.
Embodiment 113: A method for selecting the subject of any one of Embodiments 95 to 108, comprising: obtaining from a subject having the disease or disorder a sample of cells comprising a non-lineage committed CD34+ cell; and contacting the sample of cells with least one perturbagen selected from Table 3, or a variant thereof, wherein when the at least one perturbagen alters a gene signature in the sample of cells, the subject is selected as a subject.
Embodiment 114: A method for selecting the subject of any one of Embodiments 95 to 108, comprising: obtaining from a subject having the disease or disorder a sample of cells comprising a non-lineage committed CD34+ cell; and contacting the sample of cells with at least one perturbagen capable of altering a gene signature in a non-lineage committed CD34+ cell, wherein when the at least one perturbagen increases in the sample of cells the expression and/or activity of one or more genes selected from the genes designated as an “up” gene in the gene directionality column of Table 1a and/or Table 2 and/or decreases in the sample of cells the expression and/or activity of one or more genes selected from the genes designated as a “down” gene in the gene directionality column of Table 1a and/or Table 2 and/or increases in the sample of cells the expression and/or activity of one or more genes selected from Table 1b and/or decreases in the sample of cells the expression and/or activity of one or more genes selected from Table 1b, the subject is selected as a subject.
Embodiment 115: A method for selecting the subject of any one of Embodiments 95 to 108, comprising: obtaining from a subject having the disease or disorder a sample of cells comprising a non-lineage committed CD34+ cell; and contacting the sample of cells with at least one perturbagen selected from Table 3, or a variant thereof; wherein when the at least one perturbagen increases in the sample of cells the expression and/or activity of one or more genes selected from the genes designated as an “up” gene in the gene directionality column of Table 1a and/or Table 2 and/or decreases in the sample of cells the expression and/or activity of one or more genes selected from the genes designated as a “down” gene in the gene directionality column of Table 1a and/or Table 2 and/or increases in the sample of cells the expression and/or activity of one or more genes selected from Table 1b and/or decreases in the sample of cells the expression and/or activity of one or more genes selected from Table 1b, the subject is selected as a subject.
Embodiment 116: Use of the perturbagen of Table 3, or a variant thereof in the manufacture of a medicament for treating a disease or disorder characterized an abnormal oxygen delivery or a hemoglobin deficiency.
Embodiment 117: Use of the perturbagen of Table 3, or a variant thereof in the manufacture of a medicament for treating sickle cell disease or a thalassemia.
Embodiment 118: A method of identifying a candidate perturbation for promoting the transition of a starting population of progenitor cells into erythrocytes comprising HbF or HbF-expressing progenitors thereof, the method comprising: exposing the starting population of progenitor cells to a perturbation; identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into erythrocytes comprising HbF or HbF-expressing progenitors thereof following exposure of the population of cells to the perturbation; and identifying the perturbation as a candidate perturbation for promoting the transition of a population of progenitor cells into erythrocytes comprising HbF or HbF-expressing progenitors thereof based on the perturbation signature, wherein the perturbation signature is an increase in expression and/or activity in the progenitor cell of one or more genes selected from the genes designated as an “up” gene in the gene directionality column of Table 1a and/or Table 2, and/or a decrease in expression and/or activity in the progenitor cell of one or more genes selected from the genes designated as a “down” gene in the gene directionality column of Table 1a and/or Table 2 and/or an increase in expression and/or activity in the progenitor cell of one or more genes selected from Table 1b and/or a decrease in expression and/or activity in the progenitor cell of one or more genes selected from Table 1b.
Embodiment 119: The method of Embodiment 118, wherein the perturbation signature is an increase in expression and/or activity in the progenitor cell of a network module designated in the network module column of Table 1a and/or Table 1b.
Embodiment 120: The method of Embodiment 119, wherein the perturbation signature comprises, the activation of one or more genes of the network module designated in the network module column of Table 1a and/or Table 1b comprises modulating expression and/or activity of 2 or more genes within a network module.
Embodiment 121: The method of Embodiment 118, wherein the perturbation signature is an increase in expression and/or activity in the progenitor cell of two or more genes designated as an “up” gene in the gene directionality column of Table 1a and/or Table 2.
Embodiment 122: The method of Embodiment 121, wherein the perturbation signature is a decrease in expression and/or activity in the progenitor cell of two or more genes designated as a “down” gene in the gene directionality column of Table 1a and/or Table 2.
Embodiment 123: The method of Embodiment 121 or 122, wherein the perturbation signature is an increase in expression and/or activity in the progenitor cell of one or more genes selected from Table 1b.
Embodiment 124: The method of any one of Embodiments 121-123, wherein the perturbation signature is a decrease in expression and/or activity in the progenitor cell of one or more genes selected from Table 1b.
Embodiment 125: A method for making a therapeutic agent for a disease or disorder selected from a sickle cell disease or a thalassemia or a disease or disorder characterized by an abnormal oxygen delivery or a hemoglobin deficiency, comprising: (a) identifying a candidate perturbation for therapy according to the method of Embodiment 118 and (b) formulating the candidate perturbation as a therapeutic agent for the treatment of the disease or disorder.
Embodiment 126: The method of any one of Embodiments 95-99, wherein the at least one perturbagen is administered in combination with a therapeutically effective amount of one or more additional therapeutic agents.
Embodiment 127: The method of Embodiment 126, wherein the additional therapeutic agent is hydroxyurea (HU).
Embodiment 128: A method for directing a change in cell state of a progenitor cell comprising: contacting a population of cells comprising a progenitor cell with at least one perturbagen selected from Table 3, or a variant thereof, and wherein the progenitor cell is a non-lineage committed CD34+ cell.
Embodiment 129: The method of Embodiment 128, wherein the change in cell state provides an increase in the number of one or more of proerythroblasts, early erythroblasts, intermediate erythroblasts, late erythroblasts, reticulocytes, and erythrocytes, wherein one or more of the proerythroblasts, early erythroblasts, intermediate erythroblasts, late erythroblasts, reticulocytes, and erythrocytes expresses fetal hemoglobin (HbF).
Embodiment 130: The method of Embodiments 128 or 129, wherein the change in cell state provides an increase in F cells.
Embodiment 131: The method of Embodiment 129 or 130, wherein one or more of the proerythroblasts, early erythroblasts, intermediate erythroblasts, late erythroblasts, reticulocytes, and erythrocytes expresses HBG1 and/or HBG2
Embodiment 132: The method of Embodiment 131, wherein the increase in the number of erythrocytes comprising HbF is relative to the number of erythrocytes obtained from a population of progenitor cells that is not contacted with the at least one perturbagen.
Embodiment 133: The method of Embodiment 131, wherein the increase in the number of erythrocytes comprising HbF is relative to the population of progenitor cells prior to contacting with the at least one perturbagen.
Embodiment 134: The method of Embodiment 132 or Embodiment 133, wherein the change in cell state provides an increase in the number of erythrocytes comprising HbF.
Embodiment 135: The method of Embodiment 129, wherein the ratio of the number of erythrocytes comprising HbF to the number of progenitor cells is increased relative to the ratio obtained from a population of progenitor cells that is not contacted with the at least one perturbagen.
Embodiment 136: The method of Embodiment 129, wherein the ratio of the number of erythrocytes comprising HbF to the number of progenitor cells is increased relative to the ratio in the population of progenitor cells prior to contacting with the at least one perturbagen.
Embodiment 137: The method of any one of Embodiments 129 to 136, wherein the increase in the number of erythrocytes comprising HbF, is due in part to increased cell proliferation of the erythrocytes comprising HbF
Embodiment 138: The method of any one of Embodiments 129 to 137, wherein the increase in the number of erythrocytes comprising HbF, is due in part to an increased lifespan of the erythrocytes comprising HbF.
Embodiment 139: The method of any one of Embodiments 129 to 138, wherein the increase in the number of erythrocytes comprising HbF, is due in part to reduced cell death among the erythrocytes comprising HbF.
Embodiment 140: The method of any one of Embodiments 129 to 139, wherein the increase in the number of erythrocytes comprising HbF, is due in part to a change of cell state from progenitor cells into the erythrocyte lineage.
Embodiment 141: The method of any one of Embodiments 128 to 140, wherein the number of progenitor cells is decreased.
Embodiment 142: The method of Embodiment 141, wherein the decrease in the number of progenitor cells is due in part to decreased cell proliferation of the progenitor cells.
Embodiment 143: The method of Embodiment 141 or Embodiment 142, wherein the decrease in the number of progenitor cells is due in part to a decreased lifespan of the progenitor cells.
Embodiment 144: The method of any one of Embodiments 141 to 143, wherein the decrease in the number of progenitor cells is due in part to increased cell death among the progenitor cells.
Embodiment 145: The method of any one of Embodiments 141 to 144, wherein the decrease in the number of progenitor cells is relative to the number of progenitor cells in a population of progenitor cells that is not contacted with the at least one perturbagen.
Embodiment 146: The method of any one of Embodiments 141 to 145, wherein the decrease in the number of progenitor cells is relative to the number of progenitor cells in the population prior to contacting with the at least one perturbagen.
Embodiment 147: The method of any one of Embodiments 141 to 146, wherein the decrease in the number of progenitor cells is due to a change of cell state from a progenitor cell into the erythrocyte lineage.
Embodiment 148: The method of any one of Embodiments 128 to 140, wherein the number of progenitor cells is increased.
Embodiment 149: The method of Embodiment 148, wherein the increase in the number of progenitor cells is due in part to increased cell proliferation of the progenitor cells.
Embodiment 150: The method of Embodiment 148 or Embodiment 149, wherein the increase in the number of progenitor cells is due in part to an increased lifespan of the progenitor cells.
Embodiment 151: The method of any one of Embodiments 148 to 150, wherein the increase in the number of progenitor cells is due in part to decreased cell death among the progenitor cells.
Embodiment 152: The method of any one of Embodiments 148 to 151, wherein the increase in the number of progenitor cells is relative to the number of progenitor cells in a population of progenitor cells that is not contacted with the at least one perturbagen.
Embodiment 153: The method of any one of Embodiments 148 to 151, wherein the increase in the number of progenitor cells is relative to the number of progenitor cells in the population prior to contacting with the at least one perturbagen.
Embodiment 154: The method of any one of Embodiments 128 to 140, wherein the number of proerythroblasts, BFU-E cells, CFU-E cells, early erythroblasts, intermediate erythroblasts, late erythroblasts, reticulocytes, and/or erythrocytes is increased after contacting the population of cells comprising a CD34+ cell with the at least one perturbagen, wherein one or more of the proerythroblasts, early erythroblasts, intermediate erythroblasts, late erythroblasts, reticulocytes, and erythrocytes comprise HbF.
Embodiment 155: The method of any one of Embodiments 128 to 140, wherein the number of erythrocytes comprising HbF is increased after contacting the population of cells comprising a CD34+ cell with the at least one perturbagen.
Embodiment 156: The method of any one of Embodiments 128 to 140, wherein the number of reticulocytes comprising HbF, and/or erythrocytes comprising HbF is increased after contacting the population of cells comprising a CD34+ cell with the at least one perturbagen.
Embodiment 157: The method of Embodiment 154, wherein the ratio of the number of proerythroblasts, BFU-E cells, CFU-E cells, early erythroblasts, intermediate erythroblasts, late erythroblasts, reticulocytes, and/or erythrocytes to the number of progenitor cells is increased relative to the ratio obtained from a population of progenitor cells that is not contacted with the at least one perturbagen, wherein one or more of the proerythroblasts, early erythroblasts, intermediate erythroblasts, late erythroblasts, reticulocytes, and erythrocytes comprise HbF.
Embodiment 158: The method of Embodiment 154, wherein the ratio of the number proerythroblasts, BFU-E cells, CFU-E cells, early erythroblasts, intermediate erythroblasts, late erythroblasts, reticulocytes, and/or erythrocytes to the number of progenitor cells is increased relative to the ratio in the population of progenitor cells prior to contacting with the at least one perturbagen, wherein one or more of the proerythroblasts, early erythroblasts, intermediate erythroblasts, late erythroblasts, reticulocytes, and erythrocytes comprise HbF.
Embodiment 159: The method of any one of Embodiments 128 to 140, wherein the ratio of the number of early erythroblasts, intermediate erythroblasts, late erythroblasts, reticulocytes, and/or erythrocytes to the number of proerythroblasts cells is increased relative to the ratio obtained from a population of progenitor cells that is not contacted with the at least one perturbagen, wherein one or more of the proerythroblasts, early erythroblasts, intermediate erythroblasts, late erythroblasts, reticulocytes, and erythrocytes comprise HbF.
Embodiment 160: The method of any one of Embodiments 128 to 140, wherein the ratio of the number of early erythroblasts, intermediate erythroblasts, late erythroblasts, reticulocytes, and/or erythrocytes to the number of proerythroblasts is increased relative to the ratio in the population of progenitor cells prior to contacting with the at least one perturbagen, wherein one or more of the proerythroblasts, early erythroblasts, intermediate erythroblasts, late erythroblasts, reticulocytes, and erythrocytes comprise HbF.
Embodiment 161: The method of any one of Embodiments 128 to 140, wherein the ratio of the number of HBF-expressing early erythroblasts to the number of HBF-expressing proerythroblasts is increased relative to the ratio obtained from a population of progenitor cells that is not contacted with the at least one perturbagen.
Embodiment 162: The method of any one of Embodiments 128 to 140, wherein the ratio of the number of HBF-expressing early erythroblasts to the number of HBF-expressing proerythroblasts is increased relative to the ratio in the population of progenitor cells prior to contacting with the at least one perturbagen.
Embodiment 163: The method of any one of Embodiments 128 to 140, wherein the ratio of the number of HBF-expressing intermediate erythroblasts to the number of HBF-expressing early erythroblasts is increased relative to the ratio obtained from a population of progenitor cells that is not contacted with the at least one perturbagen.
Embodiment 164: The method of any one of Embodiments 128 to 140, wherein the ratio of the number of HBF-expressing intermediate erythroblasts to the number of HBF-expressing early erythroblasts is increased relative to the ratio in the population of progenitor cells prior to contacting with the at least one perturbagen.
Embodiment 165: The method of any one of Embodiments 128 to 140, wherein the ratio of the number of HBF-expressing late erythroblasts to the number of HBF-expressing intermediate erythroblasts is increased relative to the ratio obtained from a population of progenitor cells that is not contacted with the at least one perturbagen.
Embodiment 166: The method of any one of Embodiments 128 to 140, wherein the ratio of the number of HBF-expressing late erythroblasts to the number of HBF-expressing intermediate erythroblasts is increased relative to the ratio in the population of progenitor cells prior to contacting with the at least one perturbagen.
Embodiment 167: The method of any one of Embodiments 128 to 140, wherein the ratio of the number of r reticulocytes comprising HbF to the number of HBF-expressing late erythroblasts is increased relative to the ratio obtained from a population of progenitor cells that is not contacted with the at least one perturbagen.
Embodiment 168: The method of any one of Embodiments 128 to 140, wherein the ratio of the number of reticulocytes comprising HbF to the number of HBF-expressing late erythroblasts is increased relative to the ratio in the population of progenitor cells prior to contacting with the at least one perturbagen.
Embodiment 169: The method of any one of Embodiments 128 to 140, wherein the ratio of the number of erythrocytes comprising HbF to the number of reticulocytes comprising HbF is increased relative to the ratio obtained from a population of progenitor cells that is not contacted with the at least one perturbagen.
Embodiment 170: The method of any one of Embodiments 128 to 140, wherein the ratio of the number of erythrocytes comprising HbF to the number of reticulocytes comprising HbF is increased relative to the ratio in the population of progenitor cells prior to contacting with the at least one perturbagen.
Embodiment 171: The method of any one of Embodiments 128 to 140, wherein the ratio of the number of erythrocytes comprising HbF to HBF-expressing late erythroblasts is increased relative to the ratio obtained from a population of progenitor cells that is not contacted with the at least one perturbagen.
Embodiment 172: The method of any one of Embodiments 128 to 140, wherein the ratio of the number of erythrocytes comprising HbF to HBF-expressing late erythroblasts is increased relative to the ratio in the population of progenitor cells prior to contacting with the at least one perturbagen.
Embodiment 173: The method of any one of Embodiments 128 to 140, wherein the ratio of the number of erythrocytes comprising HbF to HBF-expressing intermediate erythroblasts is increased relative to the ratio obtained from a population of progenitor cells that is not contacted with the at least one perturbagen.
Embodiment 174: The method of any one of Embodiments 128 to 140, wherein the ratio of the number of erythrocytes comprising HbF to HBF-expressing intermediate erythroblasts is increased relative to the ratio in the population of progenitor cells prior to contacting with the at least one perturbagen.
Embodiment 175: The method of any one of Embodiments 128 to 140, wherein the ratio of the number of erythrocytes comprising HbF to HBF-expressing early erythroblasts is increased relative to the ratio obtained from a population of progenitor cells that is not contacted with the at least one perturbagen.
Embodiment 176: The method of any one of Embodiments 128 to 140, wherein the ratio of the number of erythrocytes comprising HbF to HBF-expressing early erythroblasts is increased relative to the ratio in the population of progenitor cells prior to contacting with the at least one perturbagen.
Embodiment 177: The method of any one of Embodiments 128 to 140, wherein the ratio of the number of erythrocytes comprising HbF to HBF-expressing proerythroblasts is increased relative to the ratio obtained from a population of progenitor cells that is not contacted with the at least one perturbagen.
Embodiment 178: The method of any one of Embodiments 128 to 140, wherein the ratio of the number of erythrocytes comprising HbF to HBF-expressing proerythroblasts is increased relative to the ratio in the population of progenitor cells prior to contacting with the at least one perturbagen.
Embodiment 179: The method of any one of Embodiments 128 to 140, wherein the ratio of the number of erythrocytes comprising HbF to proerythroblasts is increased relative to the ratio obtained from a population of progenitor cells that is not contacted with the at least one perturbagen.
Embodiment 180: The method of any one of Embodiments 128 to 140, wherein the ratio of the number of erythrocytes comprising HbF to proerythroblasts is increased relative to the ratio in the population of progenitor cells prior to contacting with the at least one perturbagen.
Embodiment 181: The method of any of Embodiments 128 to 180, wherein the number of proerythroblasts is decreased.
Embodiment 182: The method of any of Embodiments 128 to 180, wherein the number of HBF-negative or HBF-low proerythroblasts is decreased.
Embodiment 183: The method of any of Embodiments 128 to 180, wherein the number of HBF-negative or HBF-low early erythroblasts is decreased.
Embodiment 184: The method of any of Embodiments 128 to 180, wherein the number of HBF-negative or HBF-low intermediate erythroblasts is decreased.
Embodiment 185: The method of any of Embodiments 128 to 180, wherein the number of HBF-negative or HBF-low late erythroblasts is decreased.
Embodiment 186: The method of any of Embodiments 128 to 180, wherein the number of HBF-negative or HBF-low reticulocytes is decreased.
Embodiment 187: The method of any of Embodiments 128 to 180, wherein the number of proerythroblasts is increased.
Embodiment 188: The method of any of Embodiments 128 to 180, wherein the number of HBF-positive or HBF-high proerythroblasts is increased.
Embodiment 189: The method of any of Embodiments 128 to 180, wherein the number of HBF-positive or HBF-high early erythroblasts is increased.
Embodiment 190: The method of any of Embodiments 128 to 180, wherein the number of HBF-positive or HBF-high intermediate erythroblasts is increased.
Embodiment 191: The method of any of Embodiments 128 to 180, wherein the number of HBF-positive or HBF-high late erythroblasts is increased.
Embodiment 192: The method of any of Embodiments 128 to 180, wherein the number of HBF-positive or HBF-high reticulocytes is increased.
Embodiment 193: The method of any of Embodiments 128 to 180, wherein the number of HBF-positive or HBF-high erythrocytes is increased.
Embodiment 194: The method of any of Embodiments 128 to 180, wherein the number of F cells is increased.
Embodiment 195: The method of any of Embodiments 128 to 180, wherein the ratio of the number of F cells to non-F cells is increased relative to the ratio obtained from a population of progenitor cells that is not contacted with the at least one perturbagen.
Embodiment 196: The method of any of Embodiments 128 to 180, wherein the ratio of the number of F cells to non-F cells is increased relative to the ratio in the population of progenitor cells prior to contacting with the at least one perturbagen.
Embodiment 197: The method of any one of Embodiments 128 to 196, wherein the at least one perturbagen selected from Table 3, or a variant thereof, comprises at least 2, at least 3, at least 4, or at least 5 perturbagens selected from Table 3, or variants thereof.
Embodiment 198: The method of any one of Embodiments 128 to 197, wherein the at least one perturbagen is used in combination with one or more additional therapeutic agents.
Embodiment 199: The method of Embodiment 198, wherein the additional therapeutic agent is hydroxyurea (HU).
Embodiment 200: The method of any one of Embodiments 128 to 199, wherein contacting the population of progenitor cells occurs in vitro or ex vivo.
Embodiment 201: The method of any one of Embodiments 128 to 199, wherein contacting the population of progenitor cells occurs in vivo in a subject.
Embodiment 202: The method of Embodiment 201, wherein the subject is a human.
Embodiment 203: The method of Embodiment 202, wherein the human is an adult human.
Embodiment 204: A perturbagen for use in the method of any one of Embodiments 128 to 203.
Embodiment 205: A pharmaceutical composition comprising the perturbagen of Embodiment 204.
Embodiment 206: A method for treating a disease or disorder characterized by an abnormal oxygen delivery, comprising: (a) administering to a subject in need thereof a therapeutically effective amount of at least one perturbagen selected from Table 3, or a variant thereof, or (b) administering to a subject in need thereof a cell, the cell having been contacted with at least one perturbagen selected from Table 3, or a variant thereof.
Embodiment 207: A method for treating a disease or disorder characterized by a hemoglobin deficiency, comprising: (a) administering to a subject in need thereof a therapeutically effective amount of at least one perturbagen selected from Table 3, or a variant thereof, or (b) administering to a subject in need thereof a cell, the cell having been contacted with at least one perturbagen selected from Table 3, or a variant thereof.
Embodiment 208: A method for treating or preventing an sickle cell disease or a thalassemia, comprising: (a) administering to a subject in need thereof a therapeutically effective amount of at least one perturbagen selected from Table 3, or a variant thereof, or (b) administering to a subject in need thereof a cell, the cell having been contacted with at least one perturbagen selected from Table 3, or a variant thereof.
Embodiment 209: The method of Embodiment 207, wherein the hemoglobin deficiency is an abnormal and/or reduced oxygen delivery functionality of hemoglobin, optionally resultant from mutations in one or more hemoglobin genes, the mutation optionally being in a HBB gene.
Embodiment 210: The method of any one of Embodiments 206 to 208, wherein the administering is directed to the bone marrow of the subject.
Embodiment 211: The method of Embodiment 210, wherein the administering is via intraosseous injection or intraosseous infusion.
Embodiment 212: The method of any one of Embodiments 206 to 211, wherein the administering the cell is via intravenous injection or intravenous infusion.
Embodiment 213: The method of any one of Embodiments 206 to 212, wherein the administering is simultaneously or sequentially to one or more mobilization agents.
Embodiment 214: The method of any one of Embodiments 206 to 213 wherein the disease or disorder characterized by an abnormal oxygen delivery and/or a hemoglobin deficiency is an anemia.
Embodiment 215: The method of Embodiment 206 to 214, wherein the sickle cell disease or a thalassemia is beta-thalassemia (transfusion dependent).
Embodiment 216: The method of any one of Embodiments 206 to 206, wherein the sickle cell disease or a thalassemia is beta-thalassemia major.
Embodiment 217: The method of any one of Embodiments 206 to 206, wherein the sickle cell disease or a thalassemia is beta-thalassemia intermedia.
Embodiment 218: The method of any one of Embodiments 206 to 206, wherein the sickle cell disease or a thalassemia is beta-thalassemia minor.
Embodiment 219: The method of any one of Embodiments 206 to 206, wherein the sickle cell disease or a thalassemia is sickle cell anemia (SS), sickle hemoglobin-C disease (SC), sickle beta-plus thalassemia and sickle beta-zero thalassemia.
Embodiment 220: The method of any one of Embodiments 206 to 219, wherein the subject is selected by steps comprising: obtaining from the subject having the disease or disorder a sample of cells comprising a non-lineage committed CD34+ cell; and contacting the sample of cells with least one perturbagen selected from Table 3, or a variant thereof.
Embodiment 221: A method for selecting the subject of any one of Embodiments 206 to 219, comprising obtaining from a subject having the disease or disorder a sample of cells comprising a non-lineage committed CD34+ cell; and contacting the sample of cells with least one perturbagen selected from Table 3, or a variant thereof.
Embodiment 222: Use of the perturbagen of Table 3, or a variant thereof in the manufacture of a medicament for treating a disease or disorder characterized an abnormal oxygen delivery or a hemoglobin deficiency.
Embodiment 223: Use of the perturbagen of Table 3, or a variant thereof in the manufacture of a medicament for treating sickle cell disease or a thalassemia.
Embodiment 224: The method of any one of Embodiments 206-219, wherein the at least one perturbagen is administered in combination with a therapeutically effective amount of one or more additional therapeutic agents. Embodiment 225: The method of Embodiment 224, wherein the additional therapeutic agent is hydroxyurea (HU). Embodiments 226: A method for directing a change in cell state of a progenitor cell, comprising: contacting a population of cells comprising a progenitor cell with at least one perturbagen capable of altering a gene signature in the progenitor cell, wherein altering the gene signature comprises an increase in expression and/or activity in the progenitor cell of one or more genes selected from the genes designated as an “up” gene in the gene directionality column of Table 1a and/or Table 2 and/or a decrease in expression and/or activity in the progenitor cell of one or more genes selected from the genes designated as a “down” gene in the gene directionality column of Table 1a and/or Table 2 and/or an increase in expression and/or activity in the progenitor cell of one or more genes selected from Table 1b and/or a decrease in expression and/or activity in the progenitor cell of one or more genes selected from Table 1b; wherein the progenitor cell is a non-lineage committed CD34+ cell; the change in cell state provides an increase in the number of one or more of proerythroblasts, early erythroblasts, intermediate erythroblasts, late erythroblasts, reticulocytes, and erythrocytes, and the increase in the number of proerythroblasts, early erythroblasts, intermediate erythroblasts, late erythroblasts, reticulocytes, and/or erythrocytes is relative to the number of proerythroblasts, early erythroblasts, intermediate erythroblasts, late erythroblasts, reticulocytes, and/or erythrocytes obtained from a population of progenitor cells that is not contacted with the at least one perturbagen.
Embodiments 227: The method of Embodiment 226, wherein the at least one perturbagen is selected from Table 3, or a variant thereof.
Embodiments 228: The method of Embodiment 227, wherein the at least one perturbagen selected from Table 3, or a variant thereof, comprises at least 2, at least 3, at least 4, or at least 5 perturbagens selected from Table 3, or variants thereof.
Embodiments 229: The method of Embodiment 226, wherein one or more of the proerythroblasts, early erythroblasts, intermediate erythroblasts, late erythroblasts, reticulocytes, and erythrocytes expresses fetal hemoglobin (HbF) expresses HBG1 and/or HBG2.
Embodiments 230: The method of Embodiment 226, wherein one or more of the proerythroblasts, early erythroblasts, intermediate erythroblasts, late erythroblasts, reticulocytes, and erythrocytes expresses fetal hemoglobin (HbF).
Embodiments 231: The method of Embodiment 226, wherein the at least one perturbagen is used in combination with one or more additional therapeutic agents.
Embodiments 232: The method of Embodiment 231, wherein the additional therapeutic agent is hydroxyurea (HU).
Embodiments 233: The method of Embodiment 226, wherein the one or more genes selected from the genes designated as an “up” gene in the gene directionality column of Table 1a comprises at least one of DDIT4, EPRS, MTHFD2, EIF4EBP1, AARS, ABCC5, PHGDH, TUBB6, LSM6, EIF4G1, RNF167, CD320, CTNNAL1, GADD45A, PTK2, CFLAR, IGF2BP2, CDK1, CDC45, CDCA4, MELK, HAT1, PAK1, TSPAN6, TIMM17B, KDM5A, UBE3B, RPS5, PAICS, RPIA, KDELR2, PNP, CAST, H2AFV, ATP11B, CTNND1, ORC1, FDFT1, CDKN1B, INSIG1, IGF1R, TRAP1, TSTA3, SUZ12, CDK4, HMGCS1, LAP3, TBPL1, FAH, CCP110, APOE, IGF2R, DYRK3, MYBL2, APP, DNMT1, SMC3, HTATSF1, CAT, ACAT2, HK1, PSMD4, CLTC, MAP4K4, PROS1, DLD, SDHB, GNAS, COPS7A, MPC2, HEBP1, BLVRA, ID2, SCAND1, ETFB, MRPS16, PIN1, TRAK2, AMDHD2, PLEKHJ1, BZW2, PCNA, WDR61, RFC5, OXA1L, MCM3, CEP57, PSMF1, POLR2K, PSMD2, ATP6V1D, PSMD9, AKAP8L, GRN, SPAG7, ENOSF1, PCK2, PCCB, NOLC1, EBNA1BP2, CD58, RFC2, ASAH1, LAGE3, AKR7A2, and RSU1.
Embodiments 234: The method of Embodiment 226, wherein the one or more genes selected from the genes designated as a “down” gene in the gene directionality column of Table 1a comprises at least one of NUCB2, XBP1, CCNB1, CDC20, PLK1, CDK6, ITGB1BP1, CCNE2, PTPN6, CBR1, HLA-DRA, MAP7, SOX4, CASP3, DNAJB6, HOXA10, IL1B, ICAM3, ADGRG1, HLA-DMA, PDLIM1, PSMB8, EPB41L2, RPL39L, PYGL, CYB561, and HOMER2.
Embodiments 235: The method of Embodiment 226, wherein the one or more genes selected from the genes designated as an “up” gene in the gene directionality column of Table 2 comprises at least one of HMGA1, KLF1, KLF6, SREBF1, NFE2, ARID3A, GFI1B, KLF13, MLXIP, E2F8, MYBL2, HSF1, GMEB1, NFX1, TGIF1, KLF3, SP1, CENPX, HES6, and LIN28B.
Embodiments 236: The method of Embodiment 226, wherein the one or more genes selected from the genes designated as a “down” gene in the gene directionality column of Table 2 comprises at least one of HOXA10, XBP1, SOX4, ZNF385D, NFIC, BATF, HHEX, RARG, KDM5B, ZFX, SPI1, TEAD4, SATB1, NFIX, PLAGL1, MEF2C, ZBTB1, HOXA9, THAP5, and ZFP57.
Embodiments 237: The method of Embodiment 226, wherein the one or more genes selected from Table 1b comprises at least one of RAP1GAP, E2F2, RSRP1, RHD, RHCE, ERMAP, SLC2A1, CD58, SELENBP1, PPOX, NPL, ADIPOR1, BTG2, KLHDC8A, SDE2, GUK1, LBH, LTBP1, ZC3H6, TRAK2, STRADB, TMBIM1, DNAJB2, KAT2B, ABHD5, CPOX, RAB6B, PAQR9, SIAH2, NCEH1, KLF3, FRYL, MOB1B, HERC6, TSPAN5, GYPE, GYPB, FHDC1, CLCN3, ANKH, EPB41L4A, IRF1, CYSTM1, FAXDC2, TRIM10, TSPO2, CCND3, GTPBP2, GCLC, FOXO3, SERINC1, CITED2, JAZF1, MTURN, CD36, PNPLA8, BPGM, KDM7A, MFHAS1, CTSB, SLC25A37, BNIP3L, RNF19A, GRINA, HSF1, AQP3, CTSL, TMOD1, STOM, RXRA, OPTN, FRMD4A, STAM, MXI1, UROS, RIC8A, ILK, SOX6, CAT, YPEL4, UCP2, PPME1, ENDOD1, PTMS, CMAS, NFE2, KIF5A, RAB3IP, NUDT4, HECTD4, SDSL, RB1, PNP, DCAF11, ATP6V1D, DPF3, ZFYVE21, KIF26A, KLF13, CCNDBP1, EPB42, REXO5, ITFG1, TERF2IP, SLC7A5, EPN2,NATD1, PLEKHH3, SLC4A1, FAM117A, WIPI1, SMIM5, LPIN2, RIOK3, UBXN6, 2-Mar, JUNB, AKAP8L, UPK1A-AS1, PPP1R15A, GPCPD1, FAM210B, RBM38, TUBB1, ITSN1, GRAP2, WWC3, ALAS2, FAM122C, MOSPD1, SOX4, CLSTN1, PGM1, CD2, PHGDH, MLLT11, IFI16, FCER1A, RGS18, CD34, NENF, PLEK, IL1B, IGFBP7, INPP4B, BASP1, FYB1, CD74, SERPINB6, TUBB2B, LTB, LST1, AIF1, MPIG6B, HLA-DRA, HLA-DRB1, HLA-DMA, HLA-DPA1, HLA-DPB1, MAP7, CPVL, SCRN1, GNAI1, CYP3A4, PRKAG2, CLU, PVT1, ALDH1A1, NFIL3, DNM1, FAM69B, NPDC1, VIM, ARID5B, ZMIZ1, SESN2, GADD45A, DENND2D, FAM91A3P, S100A6, IER5, RGS16, PHLDA3, XPC, LXN, ZMAT3, CDKN1A, SESN1, PLIN2, RPS6, CDKN2A, ANKRD18A, LCN12, FAS, CTSD, HBBP1, DDB2, CTTN, SNORD15B, MDM2, PXMP2, PLEK2, RPS27L, LYRM1, RNF167, RNU4-34P, MYL4, FDXR, TNFSF9, CD70, GDF15, ECH1, BBC3, BAX, FTL, RPS5, ADA, GNAS, APOBEC3H, RHOC, CYP1B1, SUCNR1, TIPARP, HSD17B11, HLA-A, HLA-B, PSMB9, ASAH1, VPS28, HACD1, BGLT3, HBG1, HBG2, TRIM22, PRDX5, TSC22D1, RGS6, IFI27L2, B2M, ARID3A, RABAC1, and BEX1.

### EXAMPLES

### Example 1: Single Cell Gene Expression Profiling Adult and Fetal Erythroid Differentiation

To develop a high-resolution single cell map of adult and fetal erythropoiesis, mobilized peripheral blood (mPB) and cord blood (CB) CD34+ cells were thawed from 3 distinct donors, cultured and subjected to single cell gene expression profiling (GEP) at 8 time points throughout the in vitro erythroid differentiation process. In brief, cells were cultured and passaged every 2 days in serum free Expansion media (day 0-day 6) following by cultured in serum free Differentiation media (day 7-day 10) and culminating in cultured and passaging in maturation media (day 10-day 18). For sequencing, 3,000 cells were loaded into a single channel of a Chromium Next GEM Chip G and partitioned into droplets with gel beads using a Chromium controller per time point. After emulsion droplets were formed and collected, reverse transcription reactions were incubated at 53° C. for 45 min. Barcoded transcripts were purified, amplified, fragmented and ligated to indexed sequencing adapters, all according to the manufacturer's recommended protocol. Libraries were sequenced on an Illumina NextSeq550 or NovaSeq6000 with paired end reads as follows: Read1=28 cycles, i7 Index=8 cycles, i5=0 cycles (not used), Read2=89 cycles (NovaSeq) or 91 cycles (NextSeq). Cell Ranger (v3.1.0) mkfastq was used to generate demultiplexed FASTQ files from the raw sequencing data. Cell Ranger count was used to align reads to the human GRCh38 genome reference and quantify gene and UMI counts.

A dimensionality-reduced representation of the count matrix was embedded using UMAP, clustered using Leiden clustering and annotated using differential expression testing (Wilcoxon test) and comparisons to established marker genes. Proxies for cellular states are the annotated clusters, as shown in FIG. 1A. Analysis revealed a pseudo-trajectory associated with a fetal erythropoiesis program marked by high expression of HBG1 (FIG. 1B). Differential signatures for the 3 to 15 transition cell state (FIG. 1C) where the earliest branch point between the adult and fetal program was observed (Table 5) were then used to predict perturbations that would promote a high fetal hemoglobin cell state (Table 6).

TABLE 5
Signatures for 3 to 15 Transition

| | | Gene_Di- | |
|---|---|---|---|
| Gene | Gene_Entrez_ID | rectionality | Network_Module |
| DDIT4 | 54541 | up | 0 |
| EPRS | 2058 | up | 0 |
| MTHFD2 | 10797 | up | 0 |
| EIF4EBP1 | 1978 | up | 0 |
| AARS | 16 | up | 0 |
| ABCC5 | 10057 | up | 0 |
| PHGDH | 26227 | up | 0 |
| NUCB2 | 4925 | down | 0 |
| XBP1 | 7494 | down | 0 |
| TUBB6 | 84617 | up | 1 |
| LSM6 | 11157 | up | 1 |
| EIF4G1 | 1981 | up | 1 |
| RNF167 | 26001 | up | 1 |
| CD320 | 51293 | up | 1 |
| CCNB1 | 891 | down | 1 |
| CDC20 | 991 | down | 1 |
| PLK1 | 5347 | down | 1 |
| CTNNAL1 | 8727 | up | 2 |
| GADD45A | 1647 | up | 2 |
| PTK2 | 5747 | up | 2 |
| CFLAR | 8837 | up | 2 |
| IGF2BP2 | 10644 | up | 2 |
| CDK6 | 1021 | down | 2 |
| ITGB1BP1 | 9270 | down | 2 |
| CDK1 | 983 | up | 3 |
| CDC45 | 8318 | up | 3 |
| CDCA4 | 55038 | up | 3 |
| MELK | 9833 | up | 3 |
| HAT1 | 8520 | up | 3 |
| CCNE2 | 9134 | down | 3 |
| PAK1 | 5585 | up | 4 |
| TSPAN6 | 7105 | up | 4 |
| TIMM17B | 10245 | up | 4 |
| KDM5A | 5927 | up | 4 |
| UBE3B | 89910 | up | 4 |
| PTPN6 | 5777 | down | 4 |
| RPS5 | 6193 | up | 5 |
| PAICS | 10606 | up | 5 |
| RPIA | 22934 | up | 5 |
| KDELR2 | 11014 | up | 5 |
| PNP | 4860 | up | 5 |
| CBR1 | 873 | down | 5 |
| CAST | 10849 | up | 6 |
| H2AFV | 94239 | up | 6 |
| ATP11B | 23200 | up | 6 |
| CTNND1 | 1500 | up | 6 |
| ORC1 | 4998 | up | 6 |
| HLA-DRA | 3122 | down | 6 |
| FDFT1 | 2222 | up | 7 |
| CDKN1B | 1027 | up | 7 |
| INSIG1 | 3638 | up | 7 |
| IGF1R | 3480 | up | 7 |
| MAP7 | 9053 | down | 7 |
| TRAP1 | 7190 | up | 8 |
| TSTA3 | 7264 | up | 8 |
| SUZ12 | 23512 | up | 8 |
| CDK4 | 1019 | up | 8 |
| SOX4 | 6659 | down | 8 |
| HMGCS1 | 3157 | up | 9 |
| LAP3 | 51056 | up | 9 |
| TBPL1 | 9519 | up | 9 |
| FAH | 2184 | up | 9 |
| CCP110 | 9738 | up | 9 |
| APOE | 348 | up | 10 |
| IGF2R | 3482 | up | 10 |
| DYRK3 | 8444 | up | 10 |
| CASP3 | 836 | down | 10 |
| DNAJB6 | 10049 | down | 10 |
| MYBL2 | 4605 | up | 11 |
| APP | 351 | up | 11 |
| DNMT1 | 1786 | up | 11 |
| SMC3 | 9126 | up | 11 |
| HTATSF1 | 27336 | up | 11 |
| CAT | 10249 | up | 12 |
| ACAT2 | 39 | up | 12 |
| HOXA10 | 3206 | down | 12 |
| IL1B | 3553 | down | 12 |
| HK1 | 3098 | up | 13 |
| PSMD4 | 5710 | up | 13 |
| CLTC | 1213 | up | 13 |
| MAP4K4 | 9448 | up | 13 |
| PROS1 | 5627 | up | 14 |
| DLD | 1738 | up | 14 |
| SDHB | 6390 | up | 14 |
| ICAM3 | 3385 | down | 14 |
| GNAS | 2778 | up | 15 |
| COPS7A | 50813 | up | 15 |
| ADGRG1 | 9289 | down | 15 |
| HLA-DMA | 3108 | down | 15 |
| MPC2 | 25874 | up | 16 |
| HEBP1 | 50865 | up | 16 |
| BLVRA | 644 | up | 16 |
| ID2 | 3398 | up | 16 |
| SCAND1 | 51282 | up | 17 |
| ETFB | 2109 | up | 17 |
| MRPS16 | 51021 | up | 17 |
| PIN1 | 5300 | up | 18 |
| TRAK2 | 66008 | up | 18 |
| AMDHD2 | 51005 | up | 18 |
| PLEKHJ1 | 55111 | up | 19 |
| BZW2 | 28969 | up | 19 |
| PDLIM1 | 9124 | down | 19 |
| PCNA | 5111 | up | 20 |
| WDR61 | 80349 | up | 20 |
| RFC5 | 5985 | up | 20 |
| OXA1L | 5018 | up | 21 |
| MCM3 | 4172 | up | 21 |
| PSMB8 | 5696 | down | 21 |
| CEP57 | 9702 | up | 22 |
| PSMF1 | 9491 | up | 22 |
| POLR2K | 5440 | up | 22 |
| PSMD2 | 5708 | up | 23 |
| ATP6V1D | 51382 | up | 23 |
| PSMD9 | 5715 | up | 23 |
| AKAP8L | 26993 | up | 24 |
| GRN | 2896 | up | 24 |
| SPAG7 | 9552 | up | 24 |
| ENOSF1 | 55556 | up | 25 |
| PCK2 | 5106 | up | 25 |
| PCCB | 5096 | up | 25 |
| NOLC1 | 9221 | up | 26 |
| EBNA1BP2 | 10969 | up | 26 |
| EPB41L2 | 2037 | down | 26 |
| CD58 | 965 | up | 27 |
| RFC2 | 5982 | up | 27 |
| RPL39L | 116832 | down | 27 |
| ASAH1 | 427 | up | 28 |
| LAGE3 | 8270 | up | 28 |
| AKR7A2 | 8574 | up | 28 |
| RSU1 | 6251 | up | 29 |
| PYGL | 5836 | down | 29 |
| CYB561 | 1534 | down | 30 |
| HOMER2 | 9455 | down | 30 |

TABLE 6
Predictions for 3 to 15 Transition

| Perturbagen | Molecular | Molecular Weight | Effective in vitro | |
|---|---|---|---|---|
| No. | Formula | (g/mol) | concentration | |
| 1 | C₃₁H₃₁ClFN₇O₂ | 588.1 | 10 | μM |
| 2 | C₁₈H₁₄Cl₄N₂O | 416.1 | 0.04 | um |
| 3 | C₂₈H₃₁N₃O₆ | 505.6 | 10.0 | um |
| 4 | C₂₃H₂₇N₇O₃S₂ | 513.6 | 3.33 | um |
| 5 | C₂₂H₂₉FO₄ | 376.5 | 10 | μM |
| 6 | C₂₃H₂₈N₄O₂ | 392.5 | 10 | μM |
| 7 | C₂₅H₃₂ClFO₅ | 467 | 3.33 | um |
| 8 | C₃₀H₃₃N₇O | 507.6 | 10 | μM |
| 9 | C₄₁H₆₄O₁₄ | 780.9 | 10 | μM |
| 10 | C₁₁H₁₅NO₅ | 241.24 | 10 | μM |
| 11 | C₉H₅Cl₂NO | 214.04 | 10 | μM |
| 12 | C₁₆H₁₃N₃O₃ | 295.29 | 1 | μM |
| 13 | C₁₉H₁₉NOS | 309.4 | 10.0 | um |
| 14 | C₁₆H₁₉ClN₂ | 274.79 | 10 | μM |
| 15 | C₂₁H₂₅N₅O₄S | 443.5 | 0.12 | um |
| 16 | C₂₅H₃₁N₅O₄ | 465.5 | 10 | μM |
| 17 | C₁₃H₁₅N₃O₂S | 277.34 | 10 | μM |
| 18 | C₁₄H₁₅ClN₆O | 318.76 | 10 | μM |
| 19 | C₈H₇NO₂S | 181.21 | 10 | μM |
| 20 | C₃₂H₄₈N₄O₈ | 616.7 | 5 | μM |
| 21 | C₂₁H₁₉FN₄O | 362.4 | 3.5 | um |
| 22 | C₈H₇ClN₂O₂S | 230.67 | 10 | μM |
| 23 | C₂₃H₁₃Cl₂N₃O₂ | 434.3 | 0.04 | um |
| 24 | C₂₄H₂₉N₃O₃ | 407.5 | 10 | μM |
| 25 | C₂₀H₁₄ClN₃O₃S | 411.9 | 0.37 | um |
| 26 | C₁₉H₂₁NO₅S | 375.4 | 10 | μM |
| 27 | C₁₁H₁₁F₃N₂O₃ | 276.21 | 10.0 | um |
| 28 | C₁₄H₁₀F₃NO₂ | 281.23 | 10 | μM |
| 29 | C₂₇H₃₂N₄O₇S | 556.6 | 10 | μM |
| 30 | C₂₈H₂₅FN₆O₃ | 512.5 | 0.12 | um |
| 31 | C₃₆H₅₆O₈ | 616.8 | 10 | μM |
| 32 | C₂₀H₂₄N₂O₃ | 340.4 | 10 | μM |
| 33 | C₂₁H₂₈BN₃O₅ | 413.3 | 0.12 | um |
| 34 | C₂₇H₄₁NO₆S | 507.7 | 1.11 | um |
| 35 | C₄₇H₅₁NO₁₄ | 853.9 | 500 | nM |
| 36 | C₁₅H₂₀O₃ | 248.32 | 10 | μM |
| 37 | C₂₇H₂₉NO₁₁ | 543.5 | 3 | μM |
| 38 | C₂₀H₂₈O₃ | 316.4 | 10 | μM |
| 39 | C₂₉H₄₀N₂O₄ | 480.6 | 500 | nM |
| 40 | C₃₃H₃₄N₄O₄S | 582.7 | 0.1 | um |
| 41 | C₁₅H₂₃NO₄ | 281.35 | 0.1 | um |
| 42 | C₄₂H₆₈N₆O₆S | 785.1 | 10.0 | um |

### Example 2: Testing Perturbagens In Vitro Culture to Induce a Fetal Erythropoiesis Program (Signature Diversity Screen)

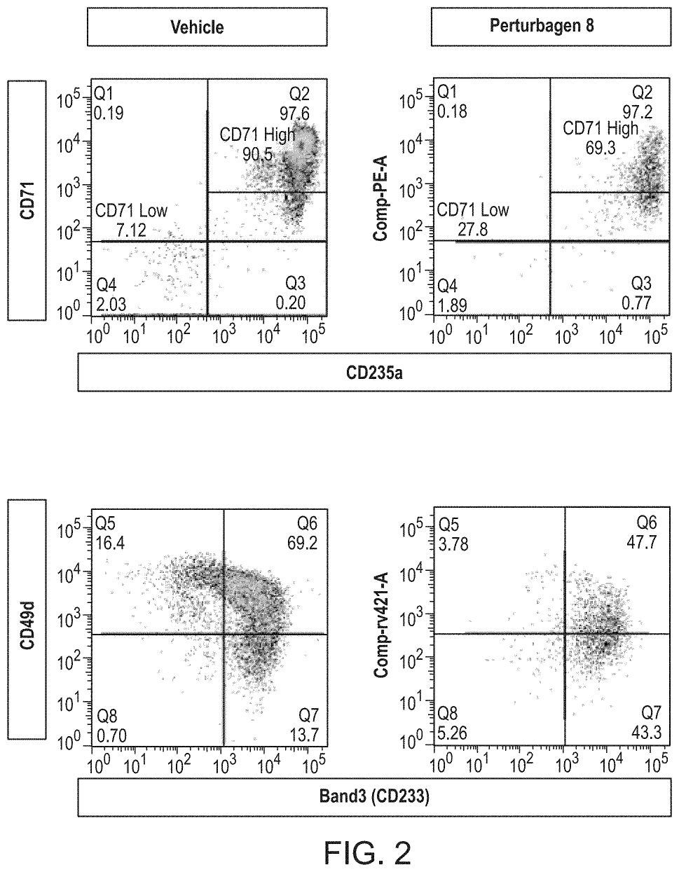

Briefly, mobilized peripheral blood (mPB), or bone marrow (BM) derived CD34+ hematopoietic stem and progenitor cells (HSPCs) were thawed and cultured in Hematopoietic stem cell expansion media (Table 7) from Day −4 to Day 0 following by 3-stage in vitro erythroid differentiation (Table 8). Specifically, cells were cultured in erythroid expansion media (phase 1) from Day 0-6, followed by differentiation media (phase 2) between Day 7-10, and culminating with incubation in erythroid maturation media (phase 3) from Day 10-18. Perturbagens reconstituted in DMSO (or appropriate solvent) were added starting at day −2 (corresponding to a stage of CD34+ hematopoietic progenitors). CD34+ cells were incubated with perturbagens for 48 hrs prior to induction of erythroid differentiation (Day 0-18) with addition of fresh compound at each cell passaging. As part of the analysis, the expansion (fold growth), viability, and erythroid maturation was measured throughout the course of differentiation. Early erythroid induction was determined by flow cytometry using a six-antibody panel (CD34, CD38, CD36, CD71, CD41, CD235a) (Table 9). Similarly, late erythroid maturation was determined by flow cytometry using a four-antibody panel (Table 10) (CD71, CD235a, CD233, CD49d) tracking increased CD233 expression, with a concomitant loss of CD49d expression, and a shift in CD71^{Hi} to CD71^{low} erythroid population (CD235a+) over 18 days (FIG. 2).

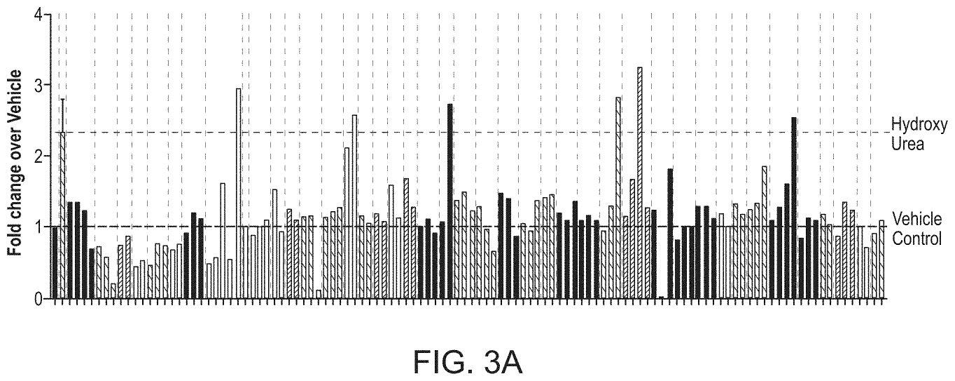

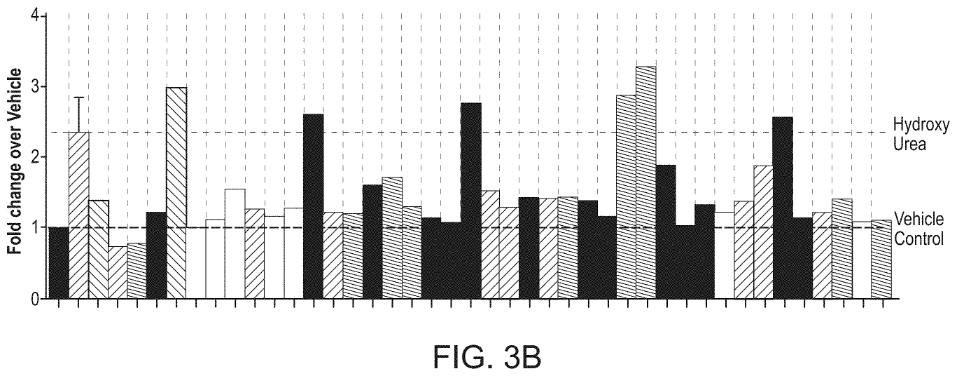

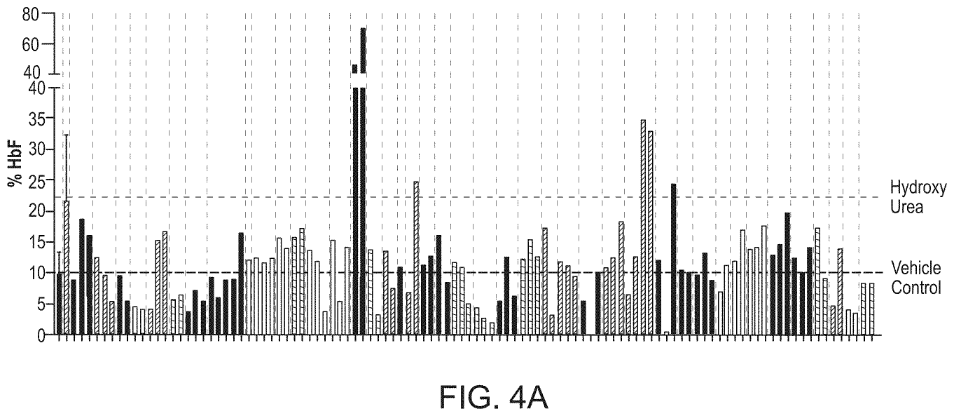

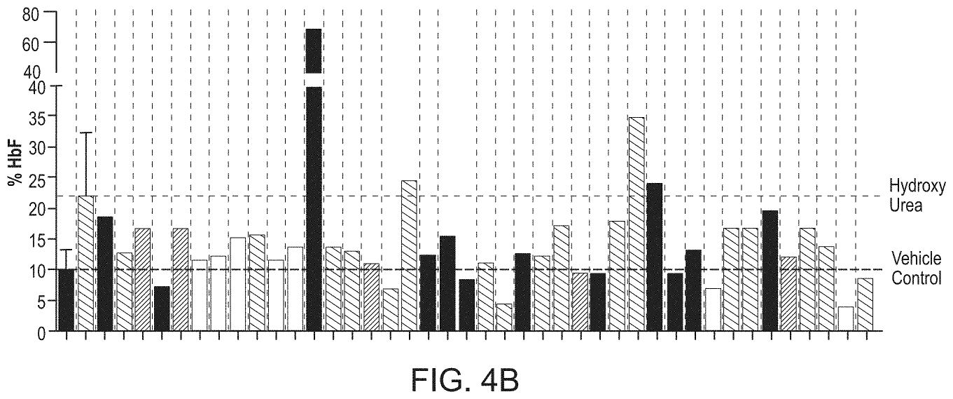

To measure the efficacy of the perturbagens at promoting a fetal hemoglobin (HbF) cell state, a flow cytometry assay was used measure the percentage of HbF expressing cells (F-cells) compared to vehicle control and hydroxyurea (50 uM) an FDA approved compound shown to increase HbF in humans. In brief, day 18 in vitro derived erythrocytes were fixed and stained with anti-HbF (gamma globin subunit) and anti-carbonic anhydrase 1 (CA1), an enzyme expressed predominantly in adult red blood cells. Furthermore, to accurately gate F-cells using flow cytometry, cord blood derived erythrocytes and Fetaltrol (Thermo Fisher) were used as positive controls. A panel of 42 compounds as listed in Table 6 are profiled for HbF induction activities using the assay protocol described above. The testing results are summarized in FIG. 3A and FIG. 3B. In addition to flow cytometry, ion-exchange chromatography is used to measure the percentage HbF relative to all other hemoglobin (HbF/HbA+HbF) in samples tested (FIG. 4A and FIG. 4B).

TABLE 7
Hematopoietic Stem Cell Progenitor Expansion Media

| Stem Cell Expansion Media | Vendor | Cat# |
|---|---|---|
| StemSpan SFEM | StemCellTech | 09650 |
| CC100 Supplement | Stem Cell Tech | 02690 |
| Human Recombinant Thrombopoietin | Stem Cell Tech | 78210 |

TABLE 8
Erythroid Differentiation Media Composition

| | Phase2 | | | |
|---|---|---|---|---|
| Phase 1 (Expansion) | (Differentiation) | Phase 3 (maturation) | Vendor | Cat# |
| StemSpan SFEM | StemSpan SFEM | StemSpan SFEM | StemCellTech | 09650 |
| GlutaMax (100x) | GlutaMax (100x) | GlutaMax (100x) | ThermoFisher | 35050061 |
| Holo-transferrin | Holo-transferrin | Holo-transferrin | Sigma | T0665 |
| (500 ug/ml) | (500 ug/ml) | (500 ug/ml) | | |
| Lipid Mixture (5 ul/ml) | Lipid Mixture (5 ul/ml) | Lipid Mixture (5 ul/ml) | Sigma | L0288 |
| Insulin (10 ng/ml) | Insulin (10 ng/ml) | Insulin (10 ng/ml) | Sigma | I9278 |
| Epo (2.5 U/ml) | Epo (2.5 U/ml) | Epo (2.5 U/ml) | R&D | 287-TC |
| SCF (50 ng/ml) | SCF (50 ng/ml) | | R&D | 255-SC050 |
| IL3 (10 ng/ml) | | | R&D | 203-IL-010 |

TABLE 9
Antibodies for Lineage Commitment

| Antibody | Fluorophore | Vendor | Clone | Cat # |
|---|---|---|---|---|
| CD41a | FITC | eBiosciences | HIP8 | 11-0419-42 |
| CD71 | PE | eBiosciences | OKT9 | 12-0719-42 |
| CD38 | AF700 | eBiosciences | HIT2 | 56-0389-42 |
| CD34 | APC-eFlur780 | eBiosciences | 4H11 | 47-0349-42 |
| CD123 | BV421 | Biolegend | 6H6 | 306018 |
| CD135 | BV421 | BD | 4G8 | 564708 |
| CD45RA | BV785 | BD | Hi100 | 563870 |
| CD235a | PE-Cy7 | BD | HIR2 | 563666 |
| CD36 | BV605 | BD | CB38 | 563518 |
| CD238 | BV510 | BD | BRIC 203 | 563475 |

TABLE 10
Antibodies for Erythroid Maturation
and Fetal Hemoglobin Detection

| Antibody | Fluorophore | Vendor | Clone | Cat # |
|---|---|---|---|---|
| CD71 | PE | eBiosciences | OKT9 | 12-0719-42 |
| CD235a | PE-Cy7 | BD | HIR2 | 563666 |
| CD49d | BV421 | BD | 9F10 | 565277 |
| CD233 (Band3) | FITC | IBGRL | BRIC6 | 9439 |
| HbF | APC | ThermoFisher | HBF-1 | MHFH05 |
| HbF | PE | IQProducts | N/A | IQP363 |
| CA | FITC | IQProducts | N/A | IQP363 |

### Example 3: Identification of Two Distinct Cell Trajectories to Induce Fetal Hemoglobin

To understand the mechanism/cell behavior driving fetal hemoglobin induction, a number of known HbF inducers were evaluated and their signature was elucidated by scRNA-seq. In brief, mPB CD34+ cells were thawed, cultured and treated with selected HbF inducers (Table 11) as described in Example 2 and subjected to single cell gene expression profiling (GEP) at 5 time points capturing the induction of erythroid differentiation from a hematopoietic stem cell progenitor. For sequencing, 10,000 cells (1000 cells/perturbation) were loaded into a single channel of a Chromium Next GEM Chip G and partitioned into droplets with gel beads using a Chromium controller per time point. After emulsion droplets were formed and collected, reverse transcription reactions were incubated at 53° C. for 45 min.

TABLE 11
Table of Perturbagens (HbF inducers) Evaluated

| | Molecular | Molecular Weight |
|---|---|---|
| Perturbagen | Formula | (g/mol) |
| A | CH₄N₂O₂ | 76.055 |
| B | C₈H₁₂N₄O₄ | 228.21 |
| C | C₂₄H₃₆N₄O₆S₂ | 540.7 |
| D | C₁₃H₁₁N₃O₄ | 273.24 |

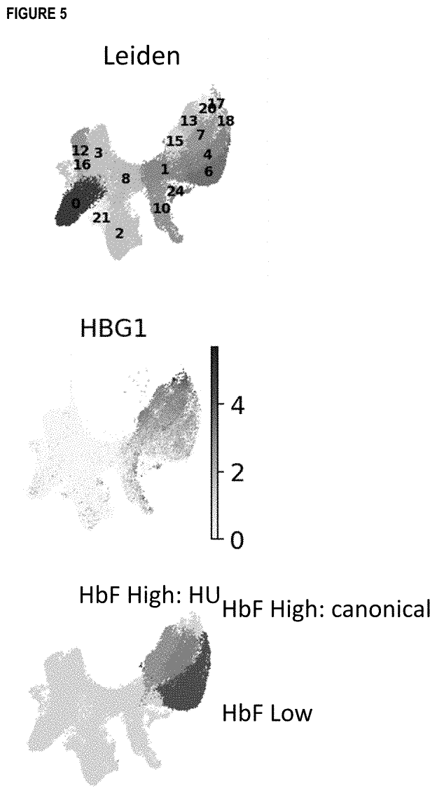

A dimensionality-reduced representation of the count matrix was embedded using UMAP, clustered using Leiden clustering and annotated using differential expression testing (Wilcoxon test) and comparisons to established marker genes. Proxies for cellular states are the annotated clusters, as shown in FIG. 5. Leiden clustering/analysis of the evaluated HbF inducers revealed two distinct pseudo-trajectories (FIG. 5) associated with high levels of HBG1/HBG2 expression. Interestingly, this gene module analysis revealed a signature associated with hydroxyurea (HU-HbF trajectory) treatments and a signature associated with both Pomalidomine treatment and BLC11A CRISPR knockout observed in control cells samples, albeit at low frequency (canonical-HbF trajectory). Based on these two trajectories, distinct signatures were generated for each transition and a list predictions were made towards each cell state.

### Example 4: Identification of a Novel Cell State Associated with High HbF and Hydroxyurea Treatment

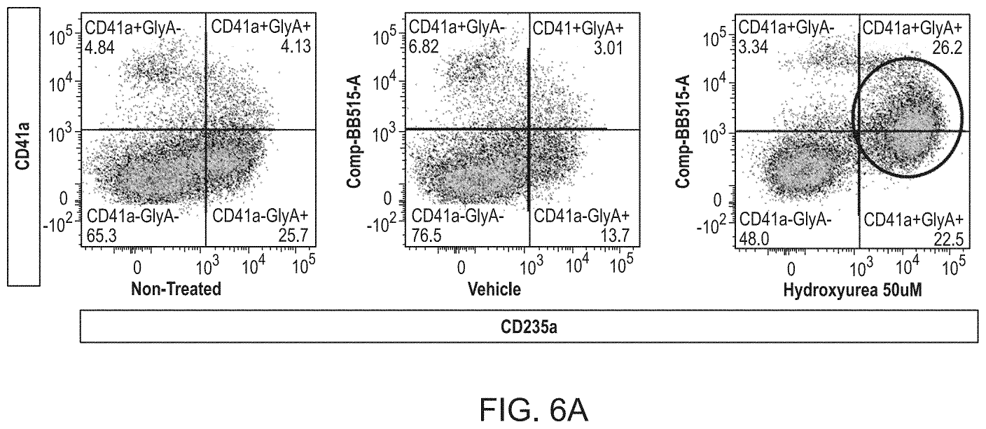

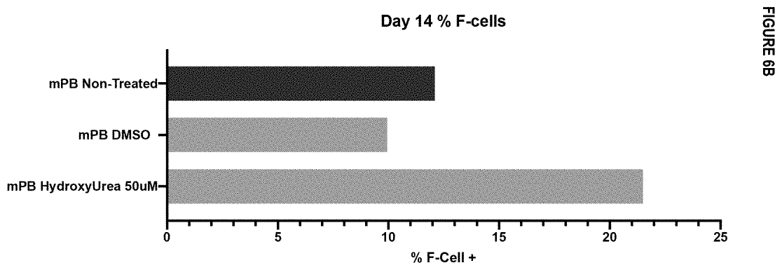

The FDA approved HbF inducer hydroxyurea (HU) was evaluated in the in vitro erythroid differentiation assay described above. Previous studies have focused on elucidated the effect of HU on erythroblast demonstrating that HU is a poor inducer of HbF in vitro. Based on the proposed reprograming of adult hematopoietic stem cells towards a fetal program, the effect of HU was evaluated on CD34+ cells directly prior to induction erythroid differentiation. Therefore, in this example, CD34+ HSPCs are cultured 4 days in stem cell maintenance media (days −4 through 0) then switched to the three phases of erythroid differentiation media. Phase 1, erythroid expansion media (days 0 through 6), Phase 2 erythroid differentiation media (days 7 through 10) and Phase 3 erythroid maturation media (days 10 through 18). Through systematic flow-based analysis of the adult CD34+ HSPCs differentiation towards the erythroid lineage, a novel cell state defined by CD34+/CD41^{Low}+/CD235a+(FIG. 6A) was observed in HU (50 uM) treatment and associated with increased number of F-cells (FIG. 6B). The aforementioned cells state induced by HU was further validated and characterized by scRNA-seq (FIG. 7) demonstrating unique gene modules associated with HbF induction and co-expression of CD34+ and CD235a+(GYPA). Table 12 shows genes that belong to HU models, as shown in FIG. 7.

TABLE 12
Genes that belong to HU modules shown in FIG. 7

| | Gene | Gene_Entrez_ID | Gene_Module |
|---|---|---|---|
| 0 | SESN2 | 83667 | 17 |
| 1 | GADD45A | 1647 | 17 |
| 2 | DENND2D | 79961 | 17 |
| 3 | FAM91A3P | | 17 |
| 4 | S100A6 | 6277 | 17 |
| 5 | IER5 | 51278 | 17 |
| 6 | RGS16 | 6004 | 17 |
| 7 | PHLDA3 | 23612 | 17 |
| 8 | XPC | 7508 | 17 |
| 9 | LXN | 56925 | 17 |
| 10 | ZMAT3 | 64393 | 17 |
| 11 | CDKN1A | 1026 | 17 |
| 12 | SESN1 | 27244 | 17 |
| 13 | PLIN2 | 123 | 17 |
| 14 | RPS6 | 6194 | 17 |
| 15 | CDKN2A | 1029 | 17 |
| 16 | ANKRD18A | 253650 | 17 |
| 17 | LCN12 | 286256 | 17 |
| 18 | FAS | 355 | 17 |
| 19 | CTSD | 1509 | 17 |
| 20 | HBBP1 | | 17 |
| 21 | DDB2 | 1643 | 17 |
| 22 | CTTN | 2017 | 17 |
| 23 | SNORD15B | | 17 |
| 24 | MDM2 | 4193 | 17 |
| 25 | PXMP2 | 5827 | 17 |
| 26 | PLEK2 | 26499 | 17 |
| 27 | RPS27L | 51065 | 17 |
| 28 | LYRM1 | 57149 | 17 |
| 29 | RNF167 | 26001 | 17 |
| 30 | RNU4-34P | | 17 |
| 31 | MYL4 | 4635 | 17 |
| 32 | FDXR | 2232 | 17 |
| 33 | TNFSF9 | 8744 | 17 |
| 34 | CD70 | 970 | 17 |
| 35 | GDF15 | 9518 | 17 |
| 36 | ECH1 | 1891 | 17 |
| 37 | BBC3 | 27113 | 17 |
| 38 | BAX | 581 | 17 |
| 39 | FTL | 2512 | 17 |
| 40 | RPS5 | 6193 | 17 |
| 41 | ADA | 100 | 17 |
| 42 | GNAS | 2778 | 17 |
| 43 | APOBEC3H | 164668 | 17 |
| 44 | CLSTN1 | 22883 | 8 |
| 45 | PGM1 | 5236 | 8 |
| 46 | CD2 | 914 | 8 |
| 47 | PHGDH | 26227 | 8 |
| 48 | MLLT11 | 10962 | 8 |
| 49 | IFI16 | 3428 | 8 |
| 50 | FCER1A | 2205 | 8 |
| 51 | RGS18 | 64407 | 8 |
| 52 | CD34 | 947 | 8 |
| 53 | NENF | 29937 | 8 |
| 54 | PLEK | 5341 | 8 |
| 55 | IL1B | 3553 | 8 |
| 56 | IGFBP7 | 3490 | 8 |
| 57 | INPP4B | 8821 | 8 |
| 58 | BASP1 | 10409 | 8 |
| 59 | FYB1 | 2533 | 8 |
| 60 | CD74 | 972 | 8 |
| 61 | SERPINB6 | 5269 | 8 |
| 62 | TUBB2B | 347733 | 8 |
| 63 | LTB | 4050 | 8 |
| 64 | LST1 | 7940 | 8 |
| 65 | AIF1 | 199 | 8 |
| 66 | MPIG6B | 80739 | 8 |
| 67 | HLA-DRA | 3122 | 8 |
| 68 | HLA-DRB1 | 3123 | 8 |
| 69 | HLA-DMA | 3108 | 8 |
| 70 | HLA-DPA1 | 3113 | 8 |
| 71 | HLA-DPB1 | 3115 | 8 |
| 72 | MAP7 | 9053 | 8 |
| 73 | CPVL | 54504 | 8 |
| 74 | SCRN1 | 9805 | 8 |
| 75 | GNAI1 | 2770 | 8 |
| 76 | CYP3A4 | 1576 | 8 |
| 77 | PRKAG2 | 51422 | 8 |
| 78 | CLU | 1191 | 8 |
| 79 | PVT1 | | 8 |
| 80 | ALDH1A1 | 216 | 8 |
| 81 | NFIL3 | 4783 | 8 |
| 82 | DNM1 | 1759 | 8 |
| 83 | FAM69B | 138311 | 8 |
| 84 | NPDC1 | 56654 | 8 |
| 85 | VIM | 7431 | 8 |
| 86 | ARID5B | 84159 | 8 |
| 87 | ZMIZ1 | 57178 | 8 |
| 88 | PRXL2A | 84293 | 8 |
| 89 | SPI1 | 6688 | 8 |
| 90 | DRAP1 | 10589 | 8 |
| 91 | PRCP | 5547 | 8 |
| 92 | PRSS23 | 11098 | 8 |
| 93 | TUBA1A | 7846 | 8 |
| 94 | FAM19A2 | 338811 | 8 |
| 95 | LYZ | 4069 | 8 |
| 96 | SLC22A17 | 51310 | 8 |
| 97 | CMTM5 | 116173 | 8 |
| 98 | NFATC4 | 4776 | 8 |
| 99 | HDC | 51696 | 8 |
| 100 | ANPEP | 290 | 8 |
| 101 | NPW | 283869 | 8 |
| 102 | ACSM3 | 6296 | 8 |
| 103 | CORO1A | 11151 | 8 |
| 104 | COTL1 | 23406 | 8 |
| 105 | CYBA | 1535 | 8 |
| 106 | ITM2BP1 | | 8 |
| 107 | ICAM3 | 3385 | 8 |
| 108 | HCST | 10870 | 8 |
| 109 | TYROBP | 7305 | 8 |
| 110 | PPP1R14A | 94274 | 8 |
| 111 | RN7SL555P | | 8 |
| 112 | APP | 351 | 8 |
| 113 | TIAM1 | 7074 | 8 |
| 114 | CBR3 | 874 | 8 |
| 115 | NCF4 | 4689 | 8 |
| 116 | CSF2RB | 1439 | 8 |
| 117 | BEX2 | 84707 | 8 |
| 118 | 6-Sep | 23157 | 8 |
| 119 | RAB33A | 9363 | 8 |
| 120 | RHOC | 389 | 54 |
| 121 | CYP1B1 | 1545 | 54 |
| 122 | SUCNR1 | 56670 | 54 |
| 123 | TIPARP | 25976 | 54 |
| 124 | HSD17B11 | 51170 | 54 |
| 125 | HLA-A | 3105 | 54 |
| 126 | HLA-B | 3106 | 54 |
| 127 | PSMB9 | 5698 | 54 |
| 128 | ASAH1 | 427 | 54 |
| 129 | VPS28 | 51160 | 54 |
| 130 | HACD1 | 9200 | 54 |
| 131 | BGLT3 | | 54 |
| 132 | HBG1 | 3047 | 54 |
| 133 | HBG2 | 3048 | 54 |
| 134 | TRIM22 | 10346 | 54 |
| 135 | PRDX5 | 25824 | 54 |
| 136 | TSC22D1 | 8848 | 54 |
| 137 | RGS6 | 9628 | 54 |
| 138 | IFI27L2 | 83982 | 54 |
| 139 | B2M | 567 | 54 |
| 140 | ARID3A | 1820 | 54 |
| 141 | RABAC1 | 10567 | 54 |
| 142 | BEX1 | 55859 | 54 |
| 143 | RAP1GAP | 5909 | 1 |
| 144 | E2F2 | 1870 | 1 |
| 145 | RSRP1 | 57035 | 1 |
| 146 | RHD | 6007 | 1 |
| 147 | RHCE | 6006 | 1 |
| 148 | ERMAP | 114625 | 1 |
| 149 | SLC2A1 | 6513 | 1 |
| 150 | CD58 | 965 | 1 |
| 151 | SELENBP1 | 8991 | 1 |
| 152 | PPOX | 3060 | 1 |
| 153 | NPL | 80896 | 1 |
| 154 | ADIPOR1 | 51094 | 1 |
| 155 | BTG2 | 7832 | 1 |
| 156 | KLHDC8A | 55220 | 1 |
| 157 | SDE2 | 163859 | 1 |
| 158 | GUK1 | 2987 | 1 |
| 159 | LBH | 221491 | 1 |
| 160 | LTBP1 | 4052 | 1 |
| 161 | ZC3H6 | 376940 | 1 |
| 162 | TRAK2 | 66008 | 1 |
| 163 | STRADB | 55437 | 1 |
| 164 | TMBIM1 | 64114 | 1 |
| 165 | DNAJB2 | 3300 | 1 |
| 166 | KAT2B | 8850 | 1 |
| 167 | ABHD5 | 51099 | 1 |
| 168 | CPOX | 1371 | 1 |
| 169 | RAB6B | 51560 | 1 |
| 170 | PAQR9 | 344838 | 1 |
| 171 | SIAH2 | 6478 | 1 |
| 172 | NCEH1 | 57552 | 1 |
| 173 | KLF3 | 51274 | 1 |
| 174 | FRYL | 285527 | 1 |
| 175 | MOB1B | 92597 | 1 |
| 176 | HERC6 | 55008 | 1 |
| 177 | TSPAN5 | 10098 | 1 |
| 178 | GYPE | 2996 | 1 |
| 179 | GYPB | 2994 | 1 |
| 180 | FHDC1 | 85462 | 1 |
| 181 | CLCN3 | 1182 | 1 |
| 182 | ANKH | 56172 | 1 |
| 183 | EPB41L4A | 64097 | 1 |
| 184 | IRF1 | 3659 | 1 |
| 185 | CYSTM1 | 84418 | 1 |
| 186 | FAXDC2 | 10826 | 1 |
| 187 | TRIM10 | 10107 | 1 |
| 188 | TSPO2 | 222642 | 1 |
| 189 | CCND3 | 896 | 1 |
| 190 | GTPBP2 | 54676 | 1 |
| 191 | GCLC | 2729 | 1 |
| 192 | FOXO3 | 2309 | 1 |
| 193 | SERINC1 | 57515 | 1 |
| 194 | CITED2 | 10370 | 1 |
| 195 | JAZF1 | 221895 | 1 |
| 196 | MTURN | 222166 | 1 |
| 197 | CD36 | 948 | 1 |
| 198 | PNPLA8 | 50640 | 1 |
| 199 | BPGM | 669 | 1 |
| 200 | KDM7A | 80853 | 1 |
| 201 | MFHAS1 | 9258 | 1 |
| 202 | CTSB | 1508 | 1 |
| 203 | SLC25A37 | 51312 | 1 |
| 204 | BNIP3L | 665 | 1 |
| 205 | RNF19A | 25897 | 1 |
| 206 | GRINA | 2907 | 1 |
| 207 | HSF1 | 3297 | 1 |
| 208 | AQP3 | 360 | 1 |
| 209 | CTSL | 1514 | 1 |
| 210 | TMOD1 | 7111 | 1 |
| 211 | STOM | 2040 | 1 |
| 212 | RXRA | 6256 | 1 |
| 213 | OPTN | 10133 | 1 |
| 214 | FRMD4A | 55691 | 1 |
| 215 | STAM | 8027 | 1 |
| 216 | MXI1 | 4601 | 1 |
| 217 | UROS | 7390 | 1 |
| 218 | RIC8A | 60626 | 1 |
| 219 | ILK | 3611 | 1 |
| 220 | SOX6 | 55553 | 1 |
| 221 | CAT | 10249 | 1 |
| 222 | YPEL4 | 219539 | 1 |
| 223 | UCP2 | 7351 | 1 |
| 224 | PPME1 | 51400 | 1 |
| 225 | ENDOD1 | 23052 | 1 |
| 226 | PTMS | 5763 | 1 |
| 227 | CMAS | 55907 | 1 |
| 228 | NFE2 | 4778 | 1 |
| 229 | KIF5A | 3798 | 1 |
| 230 | RAB3IP | 117177 | 1 |
| 231 | NUDT4 | 11163 | 1 |
| 232 | HECTD4 | 283450 | 1 |
| 233 | SDSL | 113675 | 1 |
| 234 | RB1 | 5925 | 1 |
| 235 | PNP | 4860 | 1 |
| 236 | DCAF11 | 80344 | 1 |
| 237 | ATP6V1D | 51382 | 1 |
| 238 | DPF3 | 8110 | 1 |
| 239 | ZFYVE21 | 79038 | 1 |
| 240 | KIF26A | 26153 | 1 |
| 241 | KLF13 | 51621 | 1 |
| 242 | CCNDBP1 | 23582 | 1 |
| 243 | EPB42 | 2038 | 1 |
| 244 | REXO5 | 81691 | 1 |
| 245 | ITFG1 | 81533 | 1 |
| 246 | TERF2IP | 54386 | 1 |
| 247 | SLC7A5 | 8140 | 1 |
| 248 | EPN2 | 22905 | 1 |
| 249 | NATD1 | 256302 | 1 |
| 250 | PLEKHH3 | 79990 | 1 |
| 251 | SLC4A1 | 6521 | 1 |
| 252 | FAM117A | 81558 | 1 |
| 253 | WIPI1 | 55062 | 1 |
| 254 | SMIM5 | 643008 | 1 |
| 255 | LPIN2 | 9663 | 1 |
| 256 | RIOK3 | 8780 | 1 |
| 257 | UBXN6 | 80700 | 1 |
| 258 | 2-Mar | 51257 | 1 |
| 259 | JUNB | 3726 | 1 |
| 260 | AKAP8L | 26993 | 1 |
| 261 | UPK1A-AS1 | | 1 |
| 262 | PPP1R15A | 23645 | 1 |
| 263 | GPCPD1 | 56261 | 1 |
| 264 | FAM210B | 116151 | 1 |
| 265 | RBM38 | 55544 | 1 |
| 266 | TUBB1 | 81027 | 1 |
| 267 | ITSN1 | 6453 | 1 |
| 268 | GRAP2 | 9402 | 1 |
| 269 | WWC3 | 55841 | 1 |
| 270 | ALAS2 | 212 | 1 |
| 271 | FAM122C | 159091 | 1 |
| 272 | MOSPD1 | 56180 | 1 |

### Example 5: Identification of Glucocorticoid Signaling as Pathway that Synergizes with Hydroxyurea to Increase HbF

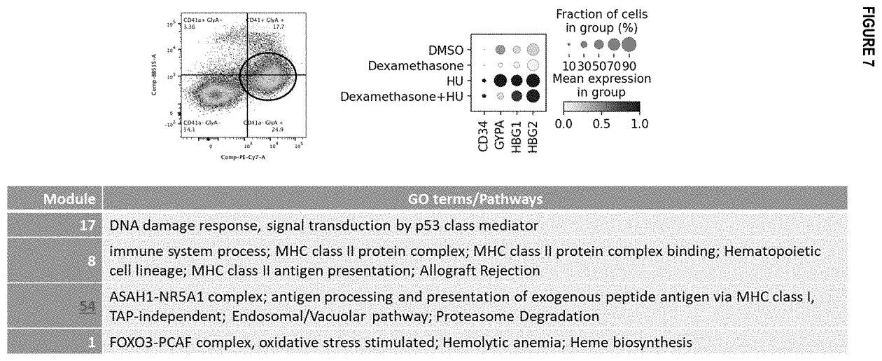

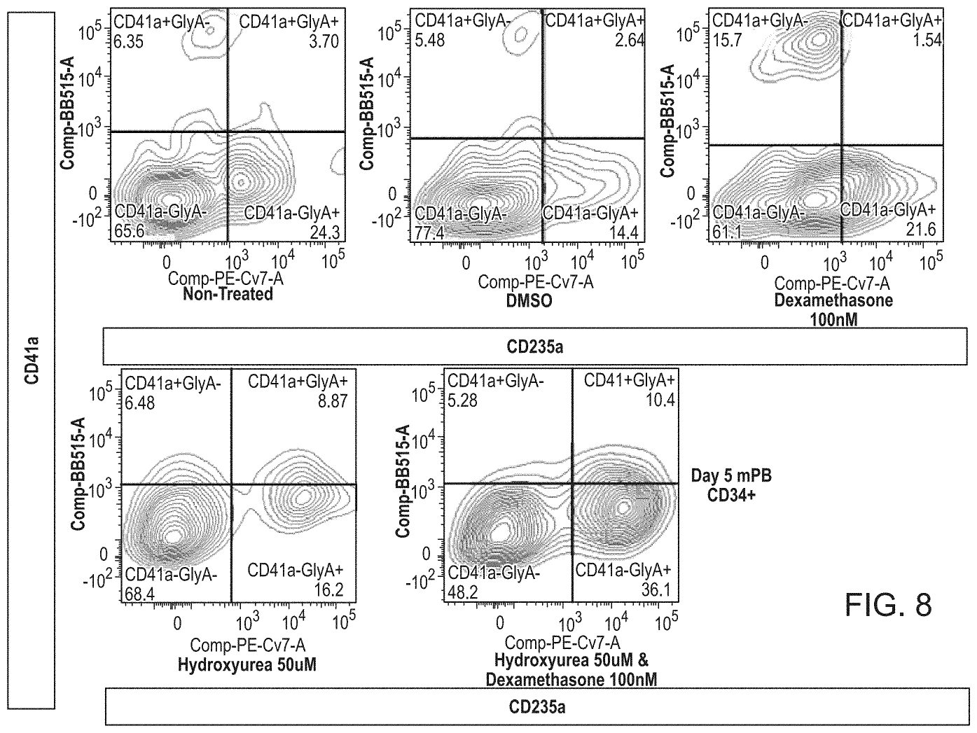

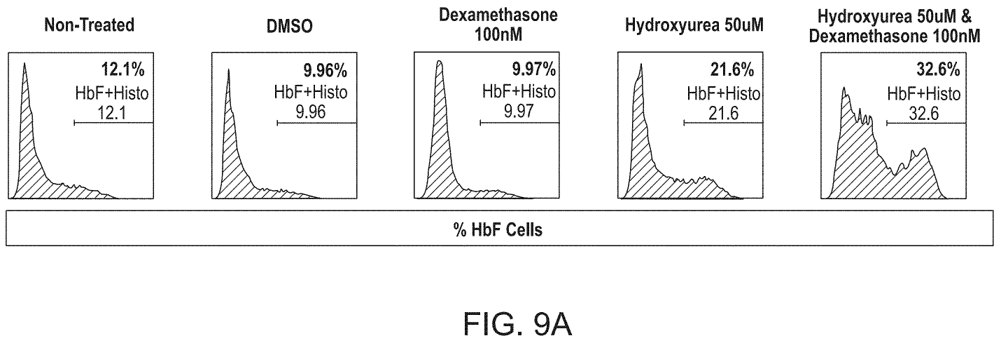

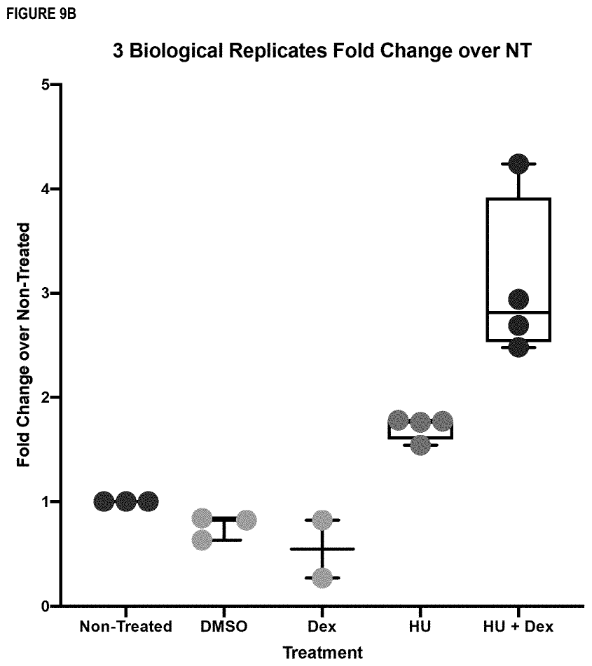

Based on the unique/novel cell state (CD34+/CD41^{Low}+/CD235a+) induced by HU treatment of human hematopoietic stem cell progenitors Applicant postulated that this cell state was reminiscent/analogous to a stress erythropoiesis cell state previously defined in mice but not well characterized in human. Interestingly, mouse stress erythropoiesis has been shown to respond to glucocorticoids. To evaluate whether HU was inducing a stress erythropoiesis cells state and whether this cell state was responsive to glucocorticoids, the effect of dexamethasone (Dex) was evaluated in the context of HU treatment. Co-treatment with HU and Dex resulted in an increase in the CD34+/CD41^{Low}+/CD235a+ cells state (FIG. 8) as determined by flow cytometry. Consistent with this observation, co-treatment with HU and Dex resulted in synergy marked by significantly higher number of F-cells compared to HU at day 14 of in vitro erythroid differentiation (FIG. 9A and FIG. 9B). Analysis of scRNA-seq of the HU and HU+Dex conditions validated the experimental data, demonstrating that HU treatment increases the expression of the glucocorticoid receptor (NR3C1) making the HU cell state responsive to glucocorticoids like Dexamethasone. In addition, treatment with Dex results in increased expression of C-Kit receptor, which is responsible for regulating hematopoietic stem cell proliferation, partially explaining the increase F-cells at the end to the culture (FIG. 10). FIG. 11A and FIG. 11B show F-cell data demonstrating that a second glucocorticoid agonist, such as Dex or Mapracorat, results in in synergy with HU as evidenced by absolute change (FIG. 11A) and fold change (FIG. 11B).

### Example 6: Identification of Small Molecules that Induce Therapeutic Levels of Fetal Hemoglobin for Treatment of Sickle Cell Disease by Pairing Machine Learning with High-Resolution Single Cell RNA Sequencing Maps of Adult and Fetal Human Erythropoiesis

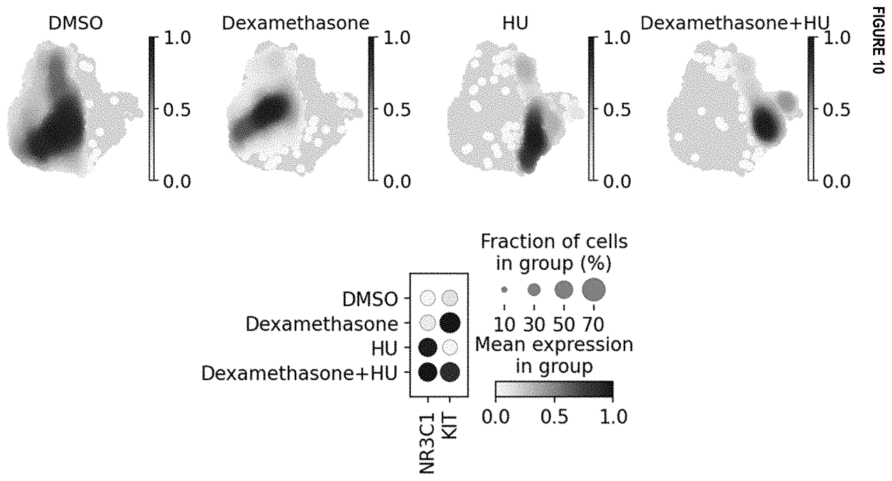

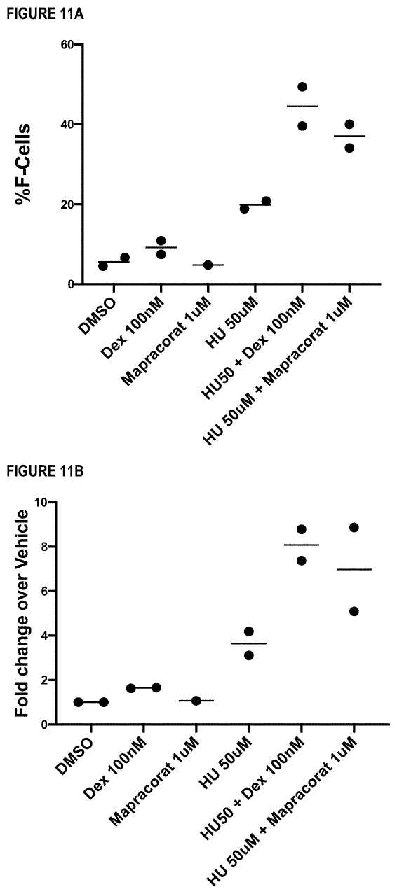

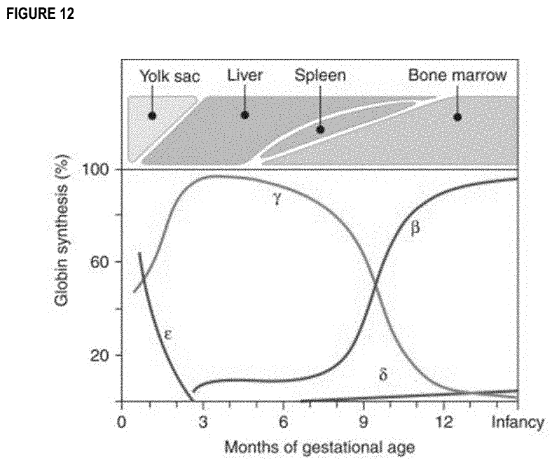

SCD and beta-thalassemia are caused by mutations in the beta globin gene. Humans undergo a fetal to adult hemoglobin switch during development (FIG. 12). The onset of disease coincides with upregulation of adult HBB gene and concomitant downregulation of HBG1/HBG2 genes. Genetic studies have shown that individuals with reactivation of fetal hemoglobin have significantly reduced disease burden in both SCD and beta-Thalassemia. A next generation small molecule with comparable efficacy to gene therapy approaches, but without the existing limitations of hydroxyurea is developed. High dimensional data and computational tools in combination with in vitro cell based assays were used to identify small molecules that can induce HbF.

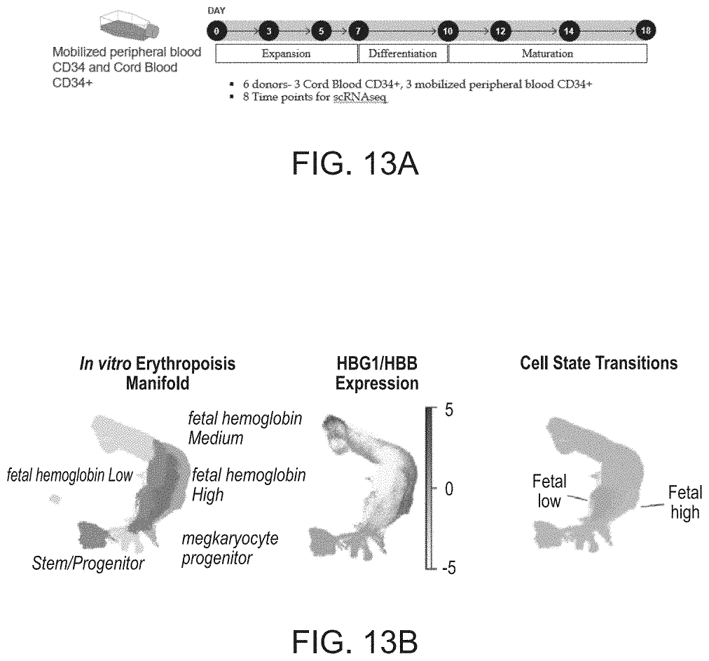

To generate a high-resolution map of adult and fetal erythropoiesis, a serum free human in vitro erythroid differentiation assay was developed to capture erythroid differentiation of adult and fetal progenitors over time using scRNA sequencing from 6 donors (3 cord blood CD34+, 3 mobilized peripheral blood CD34+) at 8 time points (FIG. 13A). A unified umap was generated to capture transcriptionally distinct pseudo-trajectories and cell states associated with fetal hemoglobin induction marked by HBG1/HBB expression (FIG. 13B).

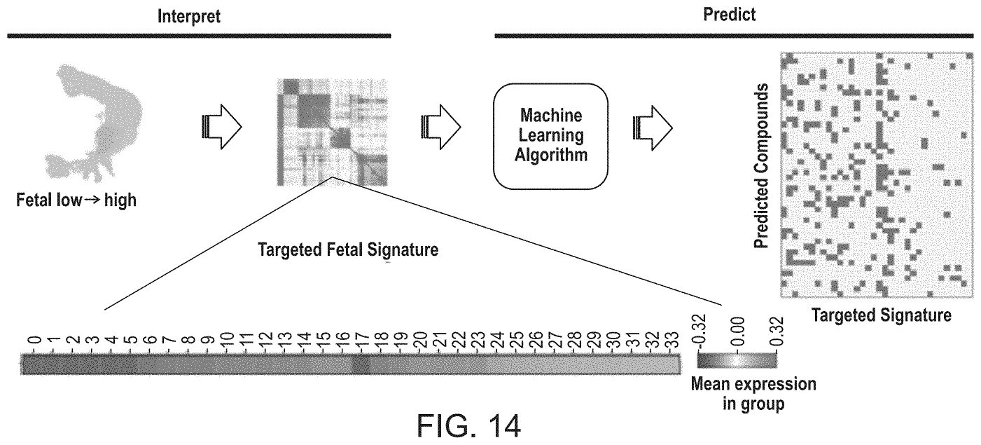

A targetable gene signature and small molecules to induce HbF was identified. 34 genes were characterized as the targetable transcriptional signature to induce high fetal cell states using in mPB identified in the cord blood. The machine learning platform predicted small molecule perturbations that target this gene signature and as a result leading to induction of HbF. The machine learning platform further stratified the targeted signatures of small molecules to cover as much diversity as possible in order to accelerate the iterative learnings in the drug discovery process (FIG. 14).

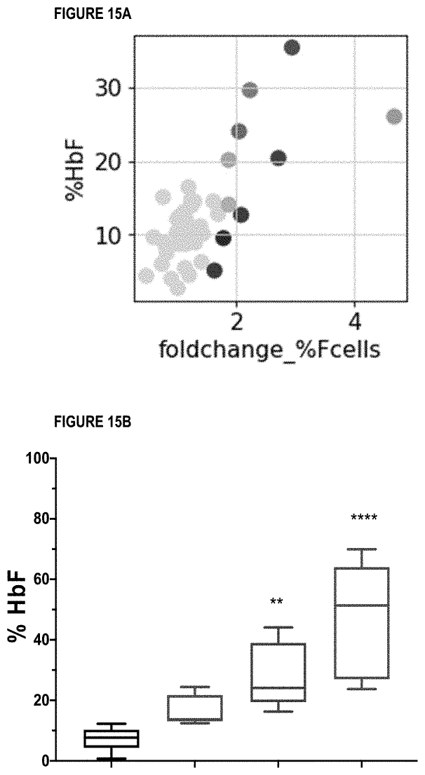

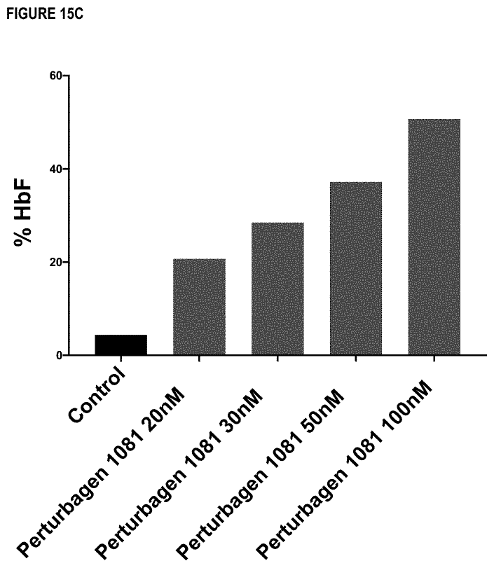

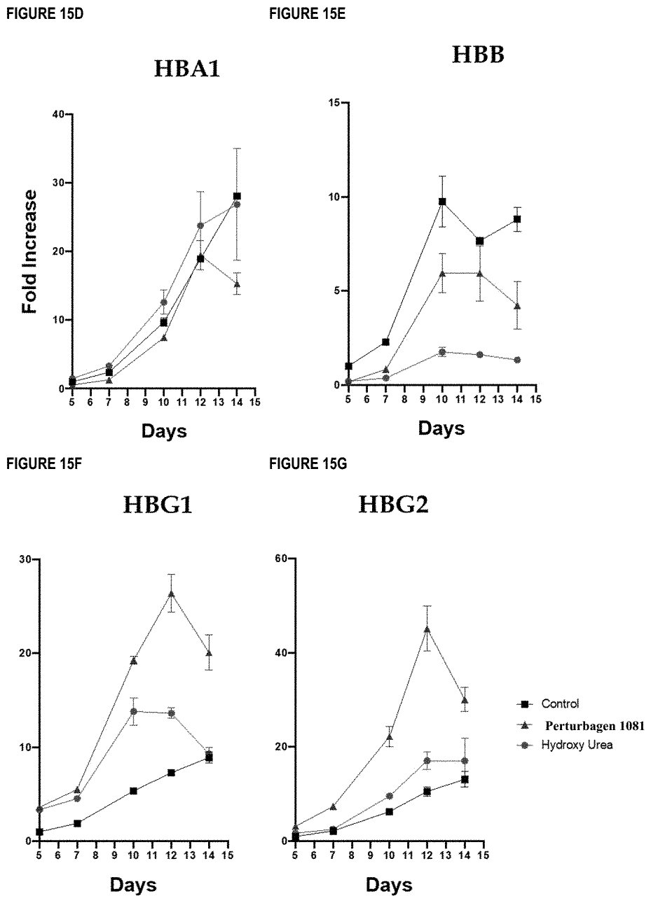

Perturbagen 1081 (C₂₁H₂₅N₅O₄S; 443.5 g/mol) was identified as an efficient HbF inducer (FIGS. 15A-15D). Adult human CD34+ progenitors were exposed to predicted perturbagens targeting a fetal erythropoiesis gene signature and their ability to induce fetal hemoglobin was measured by % HbF (HPLC) and % F-cells (flow-cytometry). Evaluation of these predicted small molecules in a 14-day human in vitro erythroid differentiation assay identified a subset that induced HbF in mobilized peripheral blood (mPB) CD34+ hematopoietic stem and progenitor cells (HSPC) from healthy donors. Dots represent average induction of each perturbation tested (n=3), identifying Perturbagen 1081 as strong as HbF inducer (FIG. 15A). HPLC analysis demonstrated that Perturbagen 1081 induced HbF (42.3%+17.26, n=4) above HU (17.16%+4.78, n=5) and BCL11A CRISPR knockdown (32.58%+10.66, n=5), ANOVA followed by Dunnett's vs Control, *p<0.05, **p<0.01, ****p<0.00001 (FIG. 15B). Exposure of CD34+ progenitors with Perturbagen 1081 demonstrate a dose dependent induction of HbF as measure by HPLC (single donor) (FIG. 15C). Temporal gene expression profiling by Nanostring revealed that Perturbagen 1081 induced robust induction of HBG1 and HBG2 and concomitant decrease in HBB expression and relative to HU (n=3, single donor) (FIGS. 15D-15G).

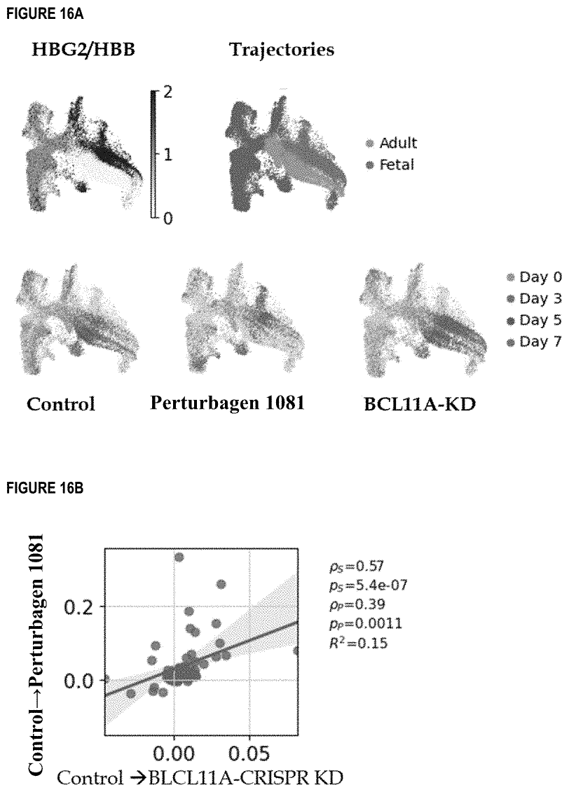

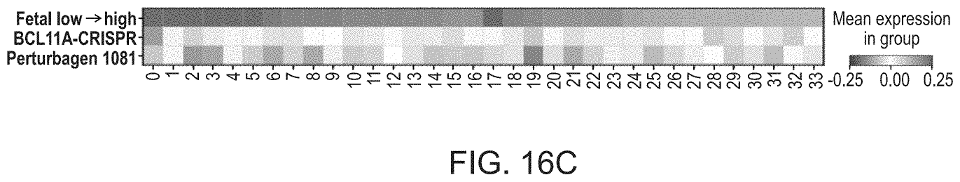

Perturbagen 1081 induced predicted gene signature associated with fetal hemoglobin induction. Perturbagen 1081 treatment reduced BCL11A expression in addition to other transcriptional changes, which elucidated a partial mechanism of action. Adult human CD34+ progenitors were treated with Perturbagen 1081 and other known HbF inducers and subjected to scRNA-seq. Analysis revealed adult and fetal pseudo-trajectories marked by HBG2/HBB differential gene expression. Perturbagen 1081 treatment and BLC11A-KD resulted in cell density shift towards the fetal trajectory (FIG. 16A). Comparison between cell responses to Perturbagen 1081 vs BCL11A-KD revealed significant transcriptional correlation (Pearson's, p-value=0.0011) between the two perturbations (FIG. 16B). Analysis of Perturbagen 1081 induced gene signature validated the fetal erythropoiesis signature predicted using a computational predictive models (FIG. 16C).

In sum, high dimensional data and computational tools identified a gene signature associated with HbF induction. The machine learning algorithm predicted and validated pertubations targeting the desired cell behavior. Perturbagen 1081 demonstrated robust HbF induction in a human in vitro system and strong transcriptional correlation with BCL11A CRISPR knockdown. Without being bound by theory, the machine learning platform augments a systems biology approach with machine learning to identify and develop drug candidates.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior disclosure.

As used herein, all headings are simply for organization and are not intended to limit the disclosure in any manner. The content of any individual section may be equally applicable to all sections.

### EQUIVALENTS

While the disclosure has been disclosed in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

1.-3. (canceled)

4. A method for directing a change in cell state of a progenitor cell, comprising:
contacting a population of cells comprising a progenitor cell with at least one perturbagen selected from Table 3, or a variant thereof, and capable of altering a gene signature in the progenitor cell,
wherein altering the gene signature comprises an increase in expression and/or activity in the progenitor cell of one or more genes selected from the genes designated as an “up” gene in the gene directionality column of Table 1a and/or Table 2 and/or a decrease in expression and/or activity in the progenitor cell of one or more genes selected from the genes designated as a “down” gene in the gene directionality column of Table 1a and/or Table 2 and/or an increase in expression and/or activity in the progenitor cell of one or more genes selected from Table 1b and/or a decrease in expression and/or activity in the progenitor cell of one or more genes selected from Table 1b and
wherein the progenitor cell is a non-lineage committed CD34+ cell.

5. The method of claim 4, wherein altering the gene signature comprises an increase in expression and/or activity in the progenitor cell of a network module designated in the network module column of Table 1a and/or Table 1b.

6. The method of claim 4, wherein the change in cell state provides an increase in the number of one or more of proerythroblasts, early erythroblasts, intermediate erythroblasts, late erythroblasts, reticulocytes, and erythrocytes.

7. The method of claim 6, wherein the change in cell state provides an increase in F cells.

8. The method of claim 6, wherein one or more of the proerythroblasts, early erythroblasts, intermediate erythroblasts, late erythroblasts, reticulocytes, and erythrocytes expresses HBG1 and/or HBG2 and/or fetal hemoglobin (HbF).

9. The method of claim 8, wherein the increase in the number of erythrocytes expressing HbF is relative to the number of erythrocytes obtained from a population of progenitor cells that is not contacted with the at least one perturbagen, or relative to the population of progenitor cells prior to contacting with the at least one perturbagen.

10. The method of claim 6, wherein the change in cell state provides an increase in the number of erythrocytes expressing HbF.

11. The method of claim 4, wherein the number of progenitor cells is decreased or increased.

12. (canceled)

13. The method of claim 6, wherein the number of proerythroblasts, BFU-E cells, CFU-E cells, early erythroblasts, intermediate erythroblasts, late erythroblasts, reticulocytes, and/or erythrocytes is increased after contacting the population of cells comprising a CD34+ cell with the at least one perturbagen, wherein one or more of the proerythroblasts, early erythroblasts, intermediate erythroblasts, late erythroblasts, reticulocytes, and erythrocytes comprise HbF.

14. The method of claim 13, wherein the number of proerythroblasts is decreased or increased.

15. (canceled)

16. The method of claim 7, wherein the number of F cells is increased.

17. The method of claim 4, wherein the at least one perturbagen selected from Table 3, or a variant thereof, comprises at least 2, at least 3, at least 4, or at least 5 perturbagens selected from Table 3, or variants thereof.

18. The method of claim 17, wherein the at least one perturbagen is used in combination with one or more additional therapeutic agents.

19. The method of claim 18, wherein the additional therapeutic agent is hydroxyurea (HU).

20.-32. (canceled)

33. A method for treating a disease or disorder characterized by an abnormal oxygen delivery, comprising:
(a) administering to a subject in need thereof a therapeutically effective amount of at least one perturbagen selected from Table 3, or a variant thereof, or
(b) administering to a subject in need thereof a cell, the cell having been contacted with at least one perturbagen selected from Table 3, or a variant thereof.

34.-47. (canceled)

48. A method of identifying a candidate perturbation for promoting the transition of a starting population of progenitor cells into erythrocytes comprising HbF or HbF-expressing progenitors thereof, the method comprising:
exposing the starting population of progenitor cells to a perturbation;
identifying a perturbation signature for the perturbation, the perturbation signature comprising one or more cellular-components and a significance score associated with each cellular-component, the significance score of each cellular-component quantifying an association between a change in expression of the cellular-component and a change in cell state of the cells in the population of progenitor cells into erythrocytes comprising HbF or HbF-expressing progenitors thereof following exposure of the population of cells to the perturbation; and
identifying the perturbation as a candidate perturbation for promoting the transition of a population of progenitor cells into erythrocytes comprising HbF or HbF-expressing progenitors thereof based on the perturbation signature,
wherein the perturbation signature is an increase in expression and/or activity in the progenitor cell of one or more genes selected from the genes designated as an “up” gene in the gene directionality column of Table 1a and/or Table 2, and/or a decrease in expression and/or activity in the progenitor cell of one or more genes selected from the genes designated as a “down” gene in the gene directionality column of Table 1a and/or Table 2 and/or an increase in expression and/or activity in the progenitor cell of one or more genes selected from Table 1b and/or a decrease in expression and/or activity in the progenitor cell of one or more genes selected from Table 1b.

49.-64. (canceled)
